(19) United States
(12) Patent Application Publication
(10) Pub. No.: US 2008/0008719 A1
(43) Pub. Date: Jan. 10, 2008

(54) Methods and compositions for the treatment of prostate cancer

(76) Inventors: Katherine S. Bowdish, Del Mar, CA (US); Hong Xin, Bonsall, CA (US); Ferda Yantiri-Wernimont, Oceanside, CA (US); Amara Siva, Oceanside, CA (US)

Correspondence Address: ROPES & GRAY LLP;PATENT DOCKETING 39/41, ONE INTERNATIONAL PLACE, BOSTON, MA, 02110-2624, US

(21) Appl. No.: 11/789,556

(22) Filed: Apr. 24, 2007

Related U.S. Application Data
(63) Continuation-in-part of application No. 11/631,911.
(60) Provisional application No. 60/586,811, filed on Jul. 10, 2004.

(51) Int. Cl. A61K 39/395; A61P 35/00; C07K 16/18

(52) U.S. Cl. 424/183.1; 530/391.7

(57) ABSTRACT

The present application relates to methods and compositions for the treatment of cancer. In specific embodiments, the application relates to the use of antibodies capable of modulating CDCP1 as therapeutic agents for the treatment of cancer and as diagnostic agents for the detection of and/or prognosis of cancer.

### RELATED APPLICATION

This application is a continuation-in-part of U.S. patent application Ser. No. 11/631,911, filed Jan. 5, 2007, which was a National Stage filing under 35 U.S.C. § 371 of PCT/US2005/024260, filed Jul. 8, 2005, which claims the benefit of U.S. Provisional Patent Application Ser. No. 60/586,811, filed Jul. 10, 2004, the disclosures of which are incorporated herein by reference in their entireties.

### TECHNICAL FIELD

This disclosure relates to antibodies which bind to the prostate cancer cell-surface antigen CDCP1. These antibodies are useful in treating prostate cancer patients.

### BACKGROUND OF RELATED ART

The promise of monoclonal antibody therapy is beginning to be realized. Efficacy has been seen in clinical trials using antibodies that target tumor cell surface antigens such as B-cell idiotypes, CD20 on malignant B cells, CD33 on leukemic blasts, and HER2/neu on breast cancer. Trastuzumab (Herceptin, anti-HER2/neu, Genentech) leads to objective responses in some metastatic breast cancer patients with overexpression of the HER2/neu oncoprotein. These exciting results provide a basis for further refinement of the existing approaches to develop new antibody-based cancer therapy strategies. Recent clinical results of monoclonal antibodies in combination with or without chemotherapy, including Erbitux (Cetuximab, C225, anti-EGFr, ImClone) in the treatment of metastatic colon cancer and Bevacizumab (Avastin, anti-vEGFr, Genentech) in the treatment of colon, renal cell cancer and other solid tumors, strongly demonstrate that monoclonal antibodies can be beneficial for cancer patients. Currently, there are multiple clinical trials with monoclonal antibodies for the treatment of prostate cancer. Generation of murine monoclonal antibodies with hybridoma technology, phage display, or other technologies, such as ribosomal display and yeast display, is especially critical for both basic and Generation of murine monoclonal antibodies with hybridoma technology, phage display, or other technologies, such as ribosomal display and yeast display, is especially critical for both basic and clinical sciences. Herceptin, Erbitux and Bevacizumab were originally screened from antigen-immunized mice.

Much research has been done to discover antibodies against cancer cells through whole cell immunization followed by screening antibodies, which bind to surface molecules of cancer cells. Although the theory of this approach is very attractive, few therapeutic antibodies were found after years of effort. This approach has proven difficult for several reasons. One reason is that the immune response in mice is not tumor specific even though cancer cells are used as an immunogen because cancer cells share a lot of common surface antigens with normal cells. Thus, the screening for tumor specific antibodies could prove to be very difficult and/or fruitless.

It is a general phenomenon that cancer cells share common antigens with normal cells. In the past, negative and positive selections have been used to screen for tumor specific antibodies. To facilitate screening for tumor specific antibodies, negative selection is a general method used to address the problem of antigens common to both normal and cancer cells, which interferes with positive selections. Numerous publications have used normal tissue cells to subtract undesired antibodies that bind to common antigens on both cancer cells and normal tissues. See, Zijlstra et al. Biochem Biophys Res Commun. 2003 Apr. 11; 303(3):733-44; Hooper et al., Oncogene. 2003 Mar. 27; 22(12): 1783-94; and Foss, Semin Oncol. 2002 June; 29(3 Suppl 7):5-11. However, most of these publications have used only one type of normal tissue cell or a couple of normal cell lines for subtraction.

Previous attempts were also made to solve this problem by an alternative method called subtractive immunization. Intensive research has been done with subtractive immunization in the past 15 years. Subtractive immunization focuses on the immunization step instead of the whole cell panning step. Subtractive immunization utilizes a distinct immune tolerization approach that can enhance the generation of monoclonal antibodies to desired antigens. Subtractive immunization is based on tolerizing the host animal to immunodominant or otherwise undesired antigens that may be structurally or functionally related to the antigens of interest. Tolerization of the host animal can be achieved through one of three methods: High Zone, Neonatal, or Drug-induced tolerization. The tolerized animal is then inoculated with the desired antigens and antibodies generated by the subsequent immune response are screened for the desired reactivity. However, a recent study suggested that neonatal “tolerization” induces immune deviation, not tolerance in the immunological sense. Neonates are not immune-privileged but generate TH2 or TH1 responses, depending on the mode of immunization. The chemical immunosuppression with cyclophosphamide was the most effective subtractive immunization technique. As those skilled in the art will appreciate, normal cell immunization followed by cyclophosphamide treatment will kill all the proliferating immune cells reactive with normal cell antigens. However, this regimen also kills all of the helper T-cells required for B-cell maturation and differentiation. Therefore, when this regimen is followed by cell immunization to elicit antibodies specific to tumor antigens, only low affinity antibodies of IgM isotype are produced.

CUB-domain-containing protein 1 (CDCP1) was first identified as an epithelial tumor antigen that was significantly overexpressed in lung cancer cell lines as compared to normal lung tissues, and also found to be highly expressed in colon adenocarcinomas (Scherl-Mostageer et al., Oncogene, 20: 4402-4408 (2001)). CDCP1 was independently identified through subtractive immunization using a highly metastatic human epidermoid carcinoma cell line against a non-metastatic variant. It was subsequently found to be highly expressed in the metastatic PC-3 prostate cancer line as well as the DLD-1 colon cancer cell line, and localized to malignant cells in colon carcinomas (Hooper et al., Oncogene, 22: 1783-1794 (2003)). Interestingly, CDCP1 is also found on CD34+CD133+ myeloid leukemic blasts, and hematopoietic stem cells (Conze et al., Ann N Y Acad Sci, 996: 222-226 (2003), Buhring et al., Stem Cells, 22: 334-343 (2004)).

It would be advantageous to have improved methods for screening antibody libraries to identify antibodies which bind to surface molecules of cancer cells. Improved methods for treating individuals suffering from cancer are also desirable. In addition, it would be advantageous to have improved antibodies that bind to a different CDCP1 antigen and which are more effective at treating prostate cancer than are the prior art antibodies.

### SUMMARY

In certain aspects, the application provides an antibody or antigen-binding fragment thereof that binds CUB-domain-containing protein 1 (CDCP1), wherein the antibody is conjugated to a cytotoxic agent. In certain embodiments, the cytotoxic agent is toxic to a CDCP1-positive cell.

In certain aspects, the application provides a method of treating prostate cancer in a mammal comprising administering to said mammal a therapeutically effective amount of an antibody that binds CDCP1, wherein the antibody is conjugated to a cytotoxin.

In certain embodiments, said antibody or antibody fragment is selected from the group consisting of a polyclonal antibody, a monoclonal antibody or antibody fragment, a recombinant antibody, a diabody, a chimerized or chimeric antibody or antibody fragment, a humanized antibody or antibody fragment, a deimmunized human antibody or antibody fragment, a fully human antibody or antibody fragment, a single chain antibody, an Fv, an Fd, an Fab, an Fab′, and an F(ab′)₂. In certain embodiments, said antibody is a monoclonal antibody.

In certain embodiments, the cytotoxic agent is selected from the group consisting of a compound that emits radiation, diphtheria A chain, nonbinding active fragments of diphtheria toxin, exotoxin A chain, ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, saponaria officinalis inhibitor, gelonin, mitogellin, restrictocin, phenomycin, enomycin and the tricothecenes, vinblastine, 4-desacetylvinblastine, vincristine, leurosidine, vindesine, or saporin. In certain embodiments, the cytotoxic agent is saporin.

In certain embodiments, said antibody is conjugated to a cytotoxic agent through a linker which releases the cytotoxic agent inside CDCP1-positive cells. In certain embodiments, said antibody or antigen-binding fragment exhibits increased effector function relative to an anti-CDCP1 antibody with a native constant region. In certain embodiments, increased effector function comprises one or more properties of the following group: a) increased antibody-dependent cell-mediated cytotoxicity (ADCC), and b) increased complement dependent cytotoxicity (CDC), compared to an anti-CDCP1 antibody with a native constant region.

In certain embodiments, said antibody has an anti-cancer activity. In certain embodiments, said anti-cancer activity is selected from the group consisting of inhibiting tumor growth, inhibiting cancer cell proliferation, inhibiting cancer cell migration, inhibiting metastasis of cancer cells, inhibiting angiogenesis, and causing tumor cell death.

In certain embodiments, said antibody blocks the interaction between CDCP1 and an interacting protein from the group consisting of N-cadherin, P-cadherin, syndecan 1, syndecan 4, or MT-SP1.

In certain embodiments, said antibody or antigen-binding fragment thereof binds the extracellular domain of CDCP1. In certain embodiments, the antibody competitively inhibits binding of a CDCP1 polypeptide to an antibody comprising a sequence selected from SEQ ID NOs:105 or 106.

In certain embodiments, said antibody or antigen-binding fragment thereof comprises a heavy chain variable region and a light chain variable region, wherein the heavy chain variable region comprises one or more CDR regions having an amino acid sequence selected from the group consisting of SEQ ID NO:71, SEQ ID NO:83, or SEQ ID NO:96, and wherein the light chain variable region comprises one or more CDR regions having an amino acid sequence selected from the group consisting of SEQ ID NO:33, SEQ ID NO:44, or SEQ ID NO:57.

In certain embodiments, said antibody or antigen-binding fragment thereof comprises a heavy chain variable region and a light chain variable region, wherein the heavy chain variable region comprises SEQ ID NO:106 and the light chain variable region comprises SEQ ID NO:105. In certain embodiments, said antibody or antigen-binding fragment thereof comprises a heavy chain and a light chain, wherein the heavy chain comprises SEQ ID NO:108 and the light chain comprises SEQ ID NO:107.

In certain embodiments, the antibody or antigen-binding fragment thereof further comprises a prostate cancer targeting agent. In certain embodiments, the targeting agent is a peptide. In certain embodiments, the targeting agent is an aptamer.

In certain embodiments, the antibody or antigen-binding fragment thereof is administered chronically to said mammal. In certain embodiments, the antibody or antigen-binding fragment thereof is administered systemically to said mammal. In certain embodiments, the antibody or antigen-binding fragment thereof is administered locally to said mammal.

In certain embodiments, the method further comprises administering a chemotherapeutic agent to said mammal. In certain embodiments, said chemotherapeutic agent and said antibody that binds CDCP1 are administered serially. In certain embodiments, said chemotherapeutic agent and said antibody that binds CDCP1 are administered simultaneously.

In certain embodiments, said cancer is prostate cancer. In certain embodiments, said mammal is a human.

The invention contemplates combinations of any of the foregoing aspects and embodiments of the invention.

### BRIEF DESCRIPTION OF THE DRAWINGS

The patent or application file contains at least one drawing executed in color. Copies of this patent or patent application publication with color drawings will be provided by the Office upon request and payment of the necessary fee.

For a fuller understanding of the subject matter described herein, reference should be made to the following detailed description, taken in connection with the accompanying diagrammatic drawings, in which:

FIGS. 1A-1C show the binding of anti-PC-3 serum to RBCs, WBCs, and PC-3 cells. (A) Post-bleed antiserum from a PC-3 immunized mouse was incubated for six rounds of RBC subtraction (S1 to S6), followed by (B) three rounds of WBC subtraction (S7 to S9), and compared to pre-bleed. (C) The binding of all RBC and WBC subtractions S1 to S9 was evaluated on PC-3 cells. Flow cytometric analysis was done with 500,000 cells per reaction; the serum dilution factor is 200.

FIGS. 2A-2C show binding of antisera derived from animals immunized with various cancer cell lines to RBCs, WBCs, and cancer cells before and after stringent subtraction. Six rounds of RBC subtraction were designated as S1 to S6, and three rounds of WBC subtraction were designated as S7 to S9. (A) Analysis of sera following each step of subtraction for RBC binding (geomean of fluorescence intensity), to determine the point at which RBC binding reached a minimum. (B) Analysis of sera following each step of subtraction for WBC binding, to determine the point at which WBC binding reached a minimum. (C) Analysis of sera following each step of subtraction for binding to the immunized cancer cell line to determine the extent of cancer cell binding maintained following stringent blood cell subtraction. Flow cytometric analyses were done with 400,000 cells per reaction, and the serum dilution factor is 200.

FIGS. 3A-3B show the amino acid sequences of antibody light and heavy chains (SEQ ID NOS:1-32) identified using the methods described. A “.” represents a glutamine (Q) resulting from readthrough of an amber stop. For the heavy chain the first amino acid shown is amino acid 2 of the variable region.

FIG. 4 shows the Western blot signatures of antibodies that bind to linear epitopes on nine cancer cell lines. L52, E23, and E27 antibodies were isolated from panning without RBC subtraction, and all others were isolated from panning with RBC subtraction. The lanes were loaded with 40 μg total protein from cell lines as follows: 1: Du145, 2: PrEC, 3: PC-3, 4: Hela, 5: MDA-MB-435, 6: KM12L4a, 7: SK-OV3, 8: A431, and 9: A-375.

FIGS. 5A-5D show relative CDCP1 message levels as determined by RT-qPCR in (A) panel of normal tissues, and (B) prostate patient samples. Norm=normal, HP=hyperplasia, PIN=prostate intraepithelial neoplasia, TMR=tumor with Gleason score ≧6; (C) prostate cancer cell lines and corresponding SCID xenografts. Values are given as fold change relative to the CDCP1 expression level of normal prostate. (D) FACS profile of chimeric 25A11 IgG on PC-3, Du145 and LNCaP cell lines. LN met is lymph node metastasis. GFI is the geometric mean of fluorescence intensity.

FIGS. 6A-6C show CDCP1 expression in prostate patient tissues. (A) Summary of IHC on five prostate patient samples. (B) IHC staining of 25A11 on 1: PC-3 cells, secondary alone (negative control), 2: PC-3 cells (positive control), 3: Patient 2, benign glands, 4: Patient 2, malignant glands, 5: Patient 5, benign glands, 6: Patient 5, malignant glands. (C) IHC analysis of CDCP1 expression in human tissues. Sections were stained with murine IgG 25A11 as primary antibody. 1: Lung, bronchus, 2: Pancreas, duct, 3: Kidney, renal tubular epithelium, 4: Heart, cardiac myocytes, 5: Spleen, fibrous trabecula, 6: Liver, hepatocytes. All photographs are at 40× magnification.

FIGS. 7A-7B. (A) CUB1 and 25A11 bind to different epitopes of CDCP1. (B) Summary of cell migration and invasion assays. PC-3 cells were treated with titrations of ch25A11 or CUB1, or 2.5 μM of PP2 and tested in Boyden chambers for inhibition of cell migration and invasion. Cells that passed through and adhered to the membrane for separate migration and invasion assays were counted for the conditions of 0.8 μM antibody concentration and 2.5 μM PP2 (average cell number is the average number of cells counted in 5 microscope fields per well). PBS in complete media (PBS-CM) was the positive control, which was assigned 100% migration or invasion value. PBS in serum-free media was the negative control for migration/invasion, in which no cells were observed to migrate/invade (data not shown).

FIG. 8 shows the internalization assay using anti-CDCP1 antibodies with appropriate saporin secondary conjugates, or with ch25A11-Sap direct conjugate. A PBS vehicle control without antibody served as blank. Media alone (no cells) controls average A490=0.114. Experiments were done in triplicate, a representative graph for the study is shown. Primary antibodies were titrated with 100 ng/well of goat anti-mouse or anti-human secondary saporin conjugates. The ch25A11 direct saporin conjugate used PBS as a vehicle control instead of secondary antibody.

FIGS. 9A-9D. (A) Table of ch25A11 pharmacokinetic parameters. Concentration of ch25A11 in the serum as a function of time: Cₜ=C₀*e^{-Ke}*^{T}, which is Cₜ=3.2736e^{-0.0034}*^{T}. Half-life in elimination phase: T_{1/2}=0.693/Kₑ. Area under the drug concentration curve: AUC=C₀/Kₑ. Concentration at time 0: C₀. Apparent volume of distribution: V_{d}=D/C₀. Clearance: Cl=Kₑ*V_{d}*1000. First-order rate constant for the elimination phase: Kₑ. Base for natural logarithms: e. Dose: D (μg). (B) Effect of ch25A11-saporin direct conjugate on PC-3 tumor growth. (C) Effect of ch25A11-saporin direct conjugate on body weight. Black arrows indicate injections on Days 7, 10, and 17; red arrow indicates primary tumor removal on Day 23. (D) Analysis of size and incidence of lymph node metastasis on Days 46 and 50.

### DETAILED DESCRIPTION OF PREFERRED EMBODIMENTS

CDCP1 is a 140-kDa glycoprotein also known as gp140, which has a single transmembrane predicted structure with three extracellular CUB (initials of the first three identified proteins containing such domains: complement factor C1r/C1s, embryonic sea urchin protein uEGF, and bone morphogenetic protein-1) domains and is a trypsin-sensitive precursor to the 80-kDa membrane glycoprotein p80. Conversion of gp140 to p80 by trypsin or serum plasmin has been shown to result in tyrosine phosphorylation on several tyrosines by Src family kinases (Brown et al., J Biol Chem, 279: 14772-14783 (2004)). Specifically, CDCP1 binds to and is phosphorylated by the Src SH2 domain and also binds to the C2 domain of PKCδ, thus forming a multi-protein complex that may play a role in cancer progression and migration (Benes et al., Cell, 121: 271-280 (2005)). In addition, CUB domains are structurally related to immunoglobulins and are thought to play important roles in cell adhesion (Duke-Cohan J S, et al., Proc. Natl. Acad. Sci. USA, 95:11336-41, (1998)). CDCP1 directly interacts with the adhesion proteins N-cadherin and P-cadherin, the matrix proteins syndecans 1 and 4, and the membrane serine protease MT-SP1, and overexpression of CDCP1 in breast cancer cells causes a loss of cell adherence phenotype (Bhatt et al., Oncogene, 24: 5333-5343 (2005)).

Stringent negative selection is used in accordance with this disclosure to screen for tumor specific antibodies. The stringent negative selection strategy in accordance with this disclosure includes multi-step subtractions with human blood cells and, optionally, normal tissue cells during the whole cell panning. The present methods significantly decrease the number of selected antibodies that bind to normal human cells, especially blood cells. These methods show improved antibody diversity by a whole cell panning approach, and provide a way to select tumor specific antibodies for cancer diagnostics and therapeutics. For therapeutic purposes, antibodies identified in accordance with the methods described herein will likely have reduced side effects on normal blood cells. This feature should improve the safety profile of the antibody for cancer therapy.

As used herein, the term “antibodies” refers to complete antibodies or antibody fragments capable of binding to a selected target. Included are Fv, scFv, Fab′ and F(ab′)₂, monoclonal and polyclonal antibodies, engineered antibodies (including chimeric, CDR-grafted and humanized, fully human antibodies, and artificially selected antibodies), and synthetic or semi-synthetic antibodies produced using phage display or alternative techniques. Small fragments, such as Fv and scFv possess advantageous properties for diagnostic and therapeutic applications on account of their small size and consequent superior tissue distribution.

The present antibodies are identified by screening an antibody library. Techniques for producing an antibody library are within the purview of one skilled in the art. See, Rader and Barbas, Phage Display, A Laboratory Manual, Cold Spring Harbor Laboratory Press, Cold Spring Harbor, N.Y. (2000), U.S. Pat. No. 6,291,161 to Lerner et al. and copending, published U.S. Patent Applications US20040072164A1 and US20040101886A1, the disclosures of which are incorporated herein in their entirety by this reference. Antibodies can be raised in a subject, for example, by one or more injections of an immunizing agent and, if desired, an adjuvant. The immunizing agent may include any type of cancer cell or fragments thereof. Typically, the immunizing agent and/or adjuvant will be injected in the subject by multiple subcutaneous or intraperitoneal injections. Suitable adjuvants include, but are not limited to, adjuvants that have been used in connection with cancer cell vaccines, such as, for example, unmethylated CpG motifs and Bacillus Calmette-Guerin (BCG). The immunization protocol may be selected by one skilled in the art without undue experimentation.

Any type of cancer cell can be used for immunizing a subject in accordance with the present methods. Suitable types of cancer cells include, but are not limited to, hematopoietic malignancies, melanoma, breast, ovarian, prostate, colon, head and neck, lung, renal, stomach, pancreatic, liver, bladder and brain. Cancer cells can be obtained from a variety of sources. For example, primary samples of cancer cells can be obtained directly from patients either through surgical techniques or biopsies. Cancer cells are also available from National Development and Research Institutes, Inc. (NDRI), New York, N.Y. Various types of cancer cells have also been deposited with and are available from American Type Culture Collection, Manassas, Va. (“ATCC”) or other depositories, such as the National Cancer Institute. Where fragments of cancer cells (such as cell membranes or mitochondria) are to be used as the immunizing agent, techniques within the purview of those skilled in the art may be employed to disrupt the cancer cells and isolate suitable components for use in immunization.

In certain embodiments, enhancement of antibody response to epitopes on the cancer cells is achieved by modification with a hapten, such as dinitrophenyl (DNP). DNP is a highly immunogenic hapten, which makes the cancer cells more easily recognized by the immune system. DNP is an aromatic compound (benzene ring with disubstituted nitro groups) that has the configuration of a hapten. A hapten is an antigenic determinant that is capable of binding to an antibody but incapable of eliciting an antibody response on its own but does when linked to a carrier protein. DNP modified autologous cancer cell vaccines have been shown to elicit a robust immune response, which is characterized by delayed type hypersensitivity, release of proinflammatory cytokines such as IFN-γ and expansion of both CD4 and CD8 T cell subsets. DNP modification of low-density antigens preferentially attracts B-cells to the site of immunogen and allows recognition and expansion of B-cells in response to DNP modified antigen. The process of B-cell trafficking to the immunogen and their subsequent expansion can be further aided by release of proinflammatory cytokines. DNP modification can be accomplished using techniques within the purview of those skilled in the art, such as those described in Berd, et al., J Clin Oncol 22:403 (2004); and Sojka, et al., Cancer Immunol Immunother 1:200 (2002).

Once an immune response is elicited in the subject, antibodies may be collected for the selection process. Cells from tissue that produce or contain antibodies are collected from the subject about three to five days after the last immunization. Suitable tissues include blood, spleen, lymph nodes and bone marrow.

Once the cells are collected, RNA is isolated therefrom using techniques known to those skilled in the art and a combinatorial antibody library is prepared. In general, techniques for preparing a combinatorial antibody library involve amplifying target sequences encoding antibodies or portions thereof, such as, for example the light and/or heavy chains using the isolated RNA of an antibody. Thus, for example, starting with a sample of antibody mRNA that is naturally diverse, first strand cDNA can be produced to provide a template. Conventional PCR or other amplification techniques can then be employed to generate the library. In certain embodiments, phage libraries expressing antibody Fab fragments (kappa or lambda light chains complexed to the IgG heavy chain fragment (Fd)) are constructed in plasmid vectors using the methods described in U.S. application Ser. No. 10/251,085, the disclosure of which is incorporated herein in its entirety by this reference.

The phage display library can then be assayed for the presence of antibodies directed against the cancer cells. Preferably, the binding specificity of antibodies is determined by an in vitro binding assay such as enzyme-linked immunosorbent assay (ELISA) and/or fluorescence-activated cell sorting (FACS). Such techniques and assays are known in the art. The binding affinity of an antibody can, for example, be determined by the Scatchard analysis of Munson and Pollard, Anal. Biochem. 107:220 (1980).

In accordance with the methods described herein, after conducting positive selection on cancer cells, human blood cells (either red, white or both), and optionally normal (i.e., non-cancerous) tissue cells are used as absorbers in conducting stringent subtractions prior to screening of the library. Suitable human normal tissue cells for use in the subtraction process include endothelial cells, epithelial cells, smooth muscle cells, and other cells isolated from such tissues as liver, lung, heart, kidney, intestine, stomach, bladder, spleen, pancreas, bone marrow, brain, thymus, prostate, ovary, testis, skin, and the like. Suitable tissue can be obtained, for example, from normal donors, late stage of fetus, or from cell lines established from these tissues.

The subtractions can be performed by contacting the library of antibodies with the normal cells and then removing the normal cells along with any antibodies bound thereto. Removal of the cells can be achieved using any technique within the purview of those skilled in the art, such as centrifuging. The supernatant containing the unbound antibodies is retained as it is the portion that contains a sub-library of antibodies that bind to cancer cells but not to normal cells. To help ensure that all antibodies that bind to normal cells are removed, multiple rounds of subtraction are performed. The multiple rounds can be conducted using the same or different types of cells. In particularly useful embodiments, at least three rounds of subtraction using red blood cells are performed. In one embodiment, subtraction is done with both red blood cells (3 rounds with different blood types (e.g., A type, B type, etc.)) and white blood cells (one round). In other embodiments, multiple subtractions are conducted using at least two types of non-cancerous cells; namely, at least one type of blood cell and at least one other type of normal tissue cells. Advantageously, the normal tissue can be derived from the same type of tissue as the cancer cells used for immunization. For example, if the subject was immunized with pancreatic cancer cells, then normal (i.e., non-cancerous) pancreatic tissue cells are used to perform the subtractions.

In conducting the negative selection, the ratio of antibody phage versus red blood cells or other absorber cells can be selected by one skilled in the art without undue experimentation. In certain embodiments, 700-1000 phage per red blood cell can be used.

To provide adequate numbers of library members, the sub-library can be amplified between rounds of subtraction and/or prior to the screening for antibodies that bind to cancer cells. Techniques for amplification are within the purview of those skilled in the art.

After the negative selection process, antibodies derived from recombinant libraries may be selected using cancer cells, or polypeptides derived therefrom, to isolate the antibodies on the basis of target specificity. As noted above, suitable techniques for selecting antibodies that bind to cancer cells are within the purview of those skilled in the art.

Hybridoma methods can also be used to identify antibodies having the desired characteristics. Such techniques are within the purview off those skilled in the art. In a hybridoma method, a mouse, rabbit, rat, hamster, or other appropriate host animal, is typically immunized with cancer cells (masked as described in copending International Application No. PCT/US2005/024261 entitled “Antibodies Against Cancer Produced Using Masked Cancer Cells As Immunogen” filed on Jul. 8, 2005, the disclosure of which is incorporated herein in its entirety) to elicit lymphocytes that produce or are capable of producing antibodies that will specifically bind to the cancer cells. Alternatively, the lymphocytes may be immunized in vitro. The lymphocytes are then fused with an immortalized cell line using a suitable fusing agent, such as polyethylene glycol, to form a hybridoma cell (See, Goding, Monoclonal Antibodies: Principles and Practice, Academic Press, (1986) pp. 59-103; Kozbor, J. Immunol., 133:3001 (1984); and Brodeur et al., Monoclonal Antibody Production Techniques and Applications, Marcel Dekker, Inc., New York, (1987) pp. 51-63 the disclosures of which are incorporated herein by this reference). The hybridoma cells are cultured in a suitable culture medium that preferably contains one or more substances that inhibit the growth or survival of the unfused, immortalized cells. The culture medium in which the hybridoma cells are cultured can then be assayed for the presence of monoclonal antibodies directed against the cancer cells using techniques within the purview of those skilled in the art (e.g., FACS analysis) and may be subjected to negative selection in accordance with the methods of the present disclosure. After the desired hybridoma cells are identified, the clones may be subcloned by limiting dilution procedures and grown by standard methods. Alternatively, the hybridoma cells may be grown in vivo as ascites in a mammal. The monoclonal antibodies secreted by the subclones are isolated or purified from the culture medium or ascites fluid by conventional immunoglobulin purification procedures.

The monoclonal antibodies that bind to cancer cells but show little or no binding to normal cells can be made by recombinant DNA methods that are within the purview of those skilled in the art. DNA encoding the monoclonal antibodies can be readily isolated and sequenced using conventional procedures (e.g., by using oligonucleotide probes that are capable of binding specifically to genes encoding the heavy and light chains of murine antibodies). The hybridoma cells or phage (depending on the particular selection method employed to identify the antibody) may serve as a preferred source of such DNA. Once isolated, the DNA may be placed into expression vectors, which are then transfected into host cells such as simian COS cells, Chinese hamster ovary (CHO) cells, or NSO or other myeloma cells that do not otherwise produce immunoglobulin protein, to obtain the synthesis of monoclonal antibodies in the recombinant host cells. The DNA also may be modified, for example, by substituting the coding sequence for human heavy and light chain constant domains in place of the homologous murine sequences or by covalently joining to the immunoglobulin coding sequence all or part of the coding sequence for a non-immunoglobulin polypeptide.

In a further embodiment, there is provided a method for identifying proteins uniquely expressed in cancer cells employing antibodies in accordance with the present disclosure, by methods well known to those skilled in the art. In one method, Fab or scFv antigens are identified by immunoprecipitation and mass spectrometry. Specifically, in one such method to identify the antigens for these antibodies, scFvs are used to immunoprecipitate the antigens from lysates prepared from the microsomal fraction of cell-surface biotinylated cancer cells. Specifically, cancer cells are labeled with a solution of 0.5 mg/ml sulfo-NHS-LC-biotin in PBS, pH8.0 for 30 seconds. After washing with PBS to remove unreacted biotin, the cells are disrupted by nitrogen cavitation and the microsomal fraction is isolated by differential centrifugation. The microsomal fraction is resuspended in NP40 Lysis Buffer and extensively precleared with normal mouse serum and protein A sepharose. Antigens are immunoprecipitated with HA-tagged scFv antibodies coupled to Rat Anti-HA agarose beads. Following immunoprecipitation, antigens are separated by SDS-PAGE and detected by Western blot using streptavidin-alkaline phosphatase (AP) or by Coomassie G-250 staining. An antibody which does not bind to the cancer cells is used as a negative control. Antigen bands are excised from the Coomassie-stained gel and identified by mass spectrometry (MS). The immunoprecipitated antigens can also be identified by matrix assisted laser desorption ionization mass spectrometry (MALDI-MS) or microcapillary reverse-phase HPLC nano-electrospray tandem mass spectrometry (μLC/MS/MS). The antigens identified can then be used as an immunogen to elicit additional antibodies thereto using techniques within the purview of those skilled in the art.

The present antibodies that bind to cancer cells but show little or no binding to normal cells in accordance with this disclosure may further include humanized antibodies or human antibodies. Humanized forms of non-human (e.g., murine) antibodies are chimeric immunoglobulins, immunoglobulin chains or fragments thereof (such as Fv, Fab, Fab′, F(ab′)₂ or other antigen-binding subsequences of antibodies) which contain minimal sequence derived from non-human immunoglobulin. Humanized antibodies include human immunoglobulins (recipient antibody) in which residues from a complementarity determining region (CDR) of the recipient are replaced by residues from a CDR of a non-human species (donor antibody) such as mouse, rat or rabbit having the desired specificity, affinity and capacity. CDR regions can be determined by one of ordinary skill in the art (see Kabat's Sequences of Proteins of Immunological Interest, 1991, 5th Ed. NIH Publication 91-3242). In some instances, Fv framework residues of the human immunoglobulin are replaced by corresponding non-human residues. Humanized antibodies may also include residues which are found neither in the recipient antibody nor in the imported CDR of framework sequences. In general, the humanized antibody will include substantially all of at least one, and typically two, variable domains, in which all or substantially all of the CDR regions correspond to those of one or more non-human immunoglobulins and all or substantially all of the FR regions are those of a human immunoglobulin consensus sequence. The humanized antibody optimally also will include at least a portion of an immunoglobulin constant region (Fc), typically that of a human immunoglobulin (Jones et al., Nature, 321:522-525 (1986); Riechmann et al., Nature, 332:323-329 (1988); and Presta, Curr. Op. Struct. Biol., 2:593-596 (1992)). Methods for humanizing non-human antibodies are well known in the art.

Generally, a humanized antibody has one or more amino acid residues introduced into it from a source which is non-human. These non-human amino acid residues are often referred to as “donor” residues, which are typically taken from a “donor” variable domain. Humanization can be essentially performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. Accordingly, such “humanized” antibodies are chimeric antibodies (U.S. Pat. No. 4,816,567), wherein substantially less than an intact human variable domain has been substituted by the corresponding sequence from a non-human species. In practice, humanized antibodies are typically human antibodies in which all or some CDR residues and possibly some FR residues are substituted by residues from analogous sites in rodent antibodies.

The present antibodies may be monovalent antibodies. Methods for preparing monovalent antibodies are well known in the art. For example, one method involves recombinant expression of immunoglobulin light chain and modified heavy chain. The heavy chain is truncated generally at any point in the Fc region so as to prevent heavy chain crosslinking. Alternatively, the relevant cysteine residues are substituted with another amino acid residue or are deleted so as to prevent crosslinking. In vitro methods are also suitable for preparing monovalent antibodies. Digestion of antibodies to produce fragments thereof, particularly, Fab fragments, can be accomplished using routine techniques known in the art. In other embodiments, bispecific antibodies are contemplated. Bispecific antibodies are monoclonal, preferably human or humanized, antibodies that have binding specificities for at least two different antigens. In the present case, one of the binding specificities is for a cancer cell, the other one is for any other antigen, and preferably for a cell-surface protein or receptor or receptor subunit. Methods for making bispecific antibodies are within the purview of those skilled in the art. Traditionally, the recombinant production of bispecific antibodies is based on the co-expression of two immunoglobulin heavy-chain/light-chain pairs, where the two heavy chains have different specificities (Milstein and Cuello, Nature, 305:537-539 (1983)). Antibody variable domains with the desired binding specificities (antibody-antigen combining sites) can be fused to immunoglobulin constant domain sequences. The fusion preferably is with an immunoglobulin heavy-chain constant domain, including at least part of the hinge, CH2, and CH3 regions. DNAs encoding the immunoglobulin heavy-chain fusions and, if desired, the immunoglobulin light chain, are inserted into separate expression vectors, and are co-transfected into a suitable host organism. For further details of illustrative currently known methods for generating bispecific antibodies see, for example, Suresh et al., Methods in Enzymology, 121:210 (1986); WO 96/27011; Brennan et al., Science 229:81 (1985); Shalaby et al., J. Exp. Med. 175:217-225 (1992); Kostelny et al., J. Immunol. 148(5):1547-1553 (1992); Hollinger et al., Proc. Natl. Acad. Sci. USA 90:6444-6448 (1993); Gruber et al., J. Immunol. 152:5368 (1994); and Tutt et al., J. Immunol. 147:60 (1991).

The present antibodies also may be utilized to detect cancerous cells in vivo. This is achieved by labeling the antibody, administering the labeled antibody to a subject, and then imaging the subject. Examples of labels useful for diagnostic imaging in accordance with the present disclosure are radiolabels such as ¹³¹I, ¹¹¹In, ¹²³I, ^{99m}Tc, ³²P, ¹²⁵I, ³H, ¹⁴C, and ¹⁸⁸Rh, fluorescent labels such as fluorescein and rhodamine, nuclear magnetic resonance active labels, positron emitting isotopes detectable by a positron emission tomography (“PET”) scanner, chemiluminescers such as luciferin, and enzymatic markers such as peroxidase or phosphatase. Short-range radiation emitters, such as isotopes detectable by short-range detector probes, such as a transrectal probe, can also be employed. These isotopes and transrectal detector probes, when used in combination, are especially useful in detecting prostatic fossa recurrences and pelvic nodal disease. The antibody can be labeled with such reagents using techniques known in the art. For example, see Wensel and Meares, Radioimmunoimaging and Radioimmunotherapy, Elsevier, N.Y. (1983), which is hereby incorporated by reference, for techniques relating to the radiolabeling of antibodies. See also, D. Colcher et al., “Use of Monoclonal Antibodies as Radiopharmaceuticals for the Localization of Human Carcinoma Xenografts in Athymic Mice”, Meth. Enzymol. 121:802-816 (1986), which is hereby incorporated by reference.

A radiolabeled antibody in accordance with this disclosure can be used for in vitro diagnostic tests. The specific activity of an antibody, binding portion thereof, probe, or interactor, depends upon the half-life, the isotopic purity of the radioactive label, and how the label is incorporated into the biological agent. In immunoassay tests, the higher the specific activity, in general, the better the sensitivity. Procedures for labeling antibodies with the radioactive isotopes are generally known in the art.

The radiolabeled antibodies can be administered to a patient where it is localized to the tumor bearing the antigen with which the antibody reacts, and is detected or “imaged” in vivo using known techniques such as radionuclear scanning using, e.g., a gamma camera or emission tomography. See e.g., A. R. Bradwell et al., “Developments in Antibody Imaging”, Monoclonal Antibodies for Cancer Detection and Therapy, R. W. Baldwin et al., (eds.), pp. 65-85 (Academic Press 1985), which is hereby incorporated by reference. Alternatively, a positron emission transaxial tomography scanner, such as designated Pet V1 located at Brookhaven National Laboratory, can be used where the radiolabel emits positrons (e.g., ¹¹C, ¹⁸F, ¹⁵O, and ¹³N).

Fluorophore and chromophore labeled biological agents can be prepared from standard moieties known in the art. Since antibodies and other proteins absorb light having wavelengths up to about 310 nm, the fluorescent moieties should be selected to have substantial absorption at wavelengths above 310 nm and preferably above 400 nm. A variety of suitable fluorescers and chromophores are described by Stryer, Science, 162:526 (1968) and Brand, L. et al., Annual Review of Biochemistry, 41:843-868 (1972), which are hereby incorporated by reference. The antibodies can be labeled with fluorescent chromophore groups by conventional procedures such as those disclosed in U.S. Pat. Nos. 3,940,475, 4,289,747, and 4,376,110, which are hereby incorporated by reference.

Antibodies of the present application are useful for the treatment of prostate cancer. The present application provides for a method of treating or preventing a cancer comprising administering to a subject in need of such treatment or prevention an effective amount of an anti-CDCP1 antibody. The anti-CDCP1 antibodies include the antibodies, antibody fragments, and antibody conjugates of the present application. Such prevention or treatment comprises inhibiting or reversing cancer cell growth or metastasis, or reducing the size of cancer or a tumor in a subject. Therapeutic methods are usually applied to human patients but may be applied to other mammals. Because the antibodies exhibit little to no binding to human blood cells or normal tissue cells, reduced side effects can be observed compared to other antibody therapies. In certain embodiments, the antibodies of the disclosure are only internalized by CDCP1-positive cells. In certain embodiments the antibodies are preferentially internalized by cancer cells. In certain embodiments the antibodies are only toxic to cells when internalized.

In certain embodiments, the application provides antibodies that bind to CDCP1. In certain embodiments, the antibodies of the application bind to the extracellular domain of CDCP1. In certain embodiments, the antibodies of the application bind to a functional domain of CDCP1. In certain embodiments, the antibodies do not bind to the same domain as the CUB1 antibody. In certain embodiments, the application provides antibodies that bind to CDCP1 isoform 1 (Genbank ID No: NP_{—}073753) and/or isoform 2 (Genbank ID No: NP_{—}835488).

In certain embodiments, cancer cells that may be treated by an anti-CDCP1 antibody include any cancer cells that exhibit CDCP1 expression or CDCP1 up-regulation. Cancers for which anti-CDCP1 therapy may be used include, for example, prostate, colon, ovarian, melanoma, myeloma, neuroblastoma, renal, breast, hematological malignancies (e.g., lymphomas and leukemias), and plasma cell cancer. Also included are any cancer cells derived from neural crest cells. In certain embodiments, antibodies used as anti-cancer therapeutics are capable of interfering with the interaction of CDCP1 and the Src SH2 domain or the C2 domain of PKCδ. Anti-CDCP1 antibodies may also target cancer cells for effector-mediated cell death.

The present antibodies can be utilized to directly kill or ablate cancerous cells in vivo. Direct killing involves administering the antibodies (which are fused to a cytotoxin) to a subject requiring such treatment. Since the antibodies recognize CDCP1 on cancer cells, any such cells to which the antibodies bind and are internalized are destroyed. Where the antibodies are used alone to kill or ablate cancer cells, such killing or ablation can be effected by initiating endogenous host immune functions, such as CDC and/or ADCC. Assays for determining whether an antibody kills cells in this manner are within the purview of those skilled in the art.

Accordingly in one embodiment, the antibodies of the present disclosure may be used to deliver a variety of cytotoxic compounds. Any cytotoxic compound can be fused to the present antibodies. The fusion can be achieved chemically or genetically (e.g., via expression as a single, fused molecule). The cytotoxic compound can be a biological, such as a polypeptide, or a small molecule. As those skilled in the art will appreciate, for small molecules, chemical fusion is used, while for biological compounds, either chemical or genetic fusion can be employed. In certain embodiments the cytotoxic agent only kills cells when internalized.

Non-limiting examples of cytotoxic compounds include therapeutic drugs, a compound emitting radiation, molecules of plant, fungal, or bacterial origin, biological proteins, and mixtures thereof. The cytotoxic drugs can be intracellularly acting cytotoxic drugs, such as short-range radiation emitters, including, for example, short-range, high-energy α-emitters. Enzymatically active toxins and fragments thereof are exemplified by diphtheria toxin A fragment, nonbinding active fragments of diphtheria toxin, exotoxin A (from Pseudomonas aeruginosa), ricin A chain, abrin A chain, modeccin A chain, α-sarcin, certain Aleurites fordii proteins, certain Dianthin proteins, Phytolacca americana proteins (PAP, PAPII and PAP-S), Morodica charantia inhibitor, curcin, crotin, Saponaria officinalis inhibitor, gelonin, mitogillin, restrictocin, phenomycin, and enomycin, for example. Procedures for preparing enzymatically active polypeptides of the immunotoxins are described in WO85/03508, which is hereby incorporated by reference. Certain cytotoxic moieties are derived from adriamycin, chlorambucil, daunomycin, methotrexate, neocarzinostatin, and platinum, for example.

Procedures for conjugating the antibodies with the cytotoxic agents have been previously described. Alternatively, the antibody can be coupled to high energy radiation emitters, for example, a radioisotope, such as ¹³¹I, a γ-emitter, which, when localized at the tumor site, results in a killing of several cell diameters. See, e.g., S. E. Order, “Analysis, Results, and Future Prospective of the Therapeutic Use of Radiolabeled Antibody in Cancer Therapy”, Monoclonal Antibodies for Cancer Detection and Therapy, R. W. Baldwin et al. (eds.), pp. 303-316 (Academic Press 1985), which is hereby incorporated by reference. Other suitable radioisotopes include α-emitters, such as ²¹²Bi, ²¹³Bi, and ²¹¹At, and β-emitters, such as ¹⁸⁶Re and ⁹⁰Y. Radiotherapy is expected to be particularly effective in connection with prostate cancer, because prostate cancer is a relatively radiosensitive tumor. Where the antibodies are used alone to kill or ablate cancer cells, such killing or ablation can be effected by initiating endogenous host immune functions, such as complement-mediated or antibody-dependent cellular cytotoxicity.

In one aspect, the present disclosure relates to methods of modulating ADCC and/or CDC of CDCP1-positive target cells by administering a murine, chimeric, humanized, or human anti-CDCP1 antibody to a subject in need thereof. The disclosure relates to variant anti-CDCP1 antibodies that elicit increased ADCC and/or CDC and to variant anti-CDCP1 antibodies that exhibit reduced or no ADCC and/or CDC activity.

In one embodiment, the variant anti-CDCP1 antibody comprises a variant or altered Fc or constant region, wherein the variant Fc or constant region exhibits increased effector function. Such said variant region may contain one or more amino acid substitutions, insertions, or deletions. Alternatively or additionally, the variant or altered Fc or constant region may comprise altered post-translational modifications, including, for example, an altered glycosylation pattern. An altered glycosylation pattern includes an increase or decrease in the number of glycosidic bonds and/or a modification in the location (i.e., amino acid residue number) of one or more glycosidic bonds.

In another embodiment, the disclosure relates to methods of eliminating CDCP1-positive cells comprising variant anti-CDCP1 antibodies that exhibit reduced or no ADCC and/or CDC activity. In one embodiment, the variant anti-CDCP1 antibody comprises a variant or altered Fc or constant region, wherein the variant Fc or constant region exhibits decreased or no effector function. Such said variant or altered Fc or constant region may contain one or more amino acid substitutions, insertions, or deletions. Alternatively or additionally, the variant Fc or constant region may comprise altered post-translational modifications, including but not limited to an altered glycosylation pattern. Examples of altered glycosylation patterns are described above.

In a further embodiment, a murine, chimeric, humanized, or human anti-CDCP1 antibody administered to a patient is a non-blocking antibody. The non-blocking anti-CDCP1 antibody may be a variant antibody as described above and may consequently exhibit modulated effector function(s). For example, a variant anti-CDCP1 antibody may not block the CDCP1 interaction with the Src SH2 domain, N-cadherin, P-cadherin, syndecan 1, syndecan 4, MT-SP1, or the C2 domain of PKCδ and may also comprise a variant constant region that elicits increased effector function, such as, e.g., increased ADCC.

In one embodiment, a variant anti-CDCP1 antibody that exhibits modulated ADCC and/or CDC activity may be administered to a subject with CDCP1-positive cancer cells. For example, a variant anti-CDCP1 antibody used in cancer therapy may exhibit enhanced effector activity compared to the parent or native antibody. In another embodiment, the variant anti-CDCP1 antibody exhibits reduced effector function, including reduced ADCC, relative to the native antibody. The said antibody may be a murine, chimeric, humanized, or human antibody. Cancers for which the variant anti-CDCP1 antibody may be used in treatment include but are not limited to prostate cancer.

The present antibodies can be administered as a therapeutic to cancer patients, especially, but not limited to, patients with prostate cancer. In one embodiment, a cancer therapy in accordance with this disclosure comprises (1) administering an anti-CDCP1 antibody that interferes with the interaction between CDCP1 and the Src SH2 domain, N-cadherin, P-cadherin, syndecan 1, syndecan 4, MT-SP1, or the C2 domain of PKCδ, thereby promoting eradication of the cancer cells; and/or administering an anti-CDCP1 antibody may directly kill the cancer cells through complement-mediated or antibody-dependent cellular cytotoxicity. In the case that CDCP1 is also expressed on normal cells, albeit at lower levels than on cancer cells, it could also be advantageous to administer an anti-CDCP1 antibody with a constant region modified to reduce or eliminate ADCC or CDC to limit damage to normal cells. For example, if CDCP1 expression is upregulated on some activated normal cells, rendering such cells vulnerable to killing by an anti-CDCP1 antibody with effector function, it may therefore also be advantageous to use an anti-CDCP1 antibody lacking effector function to avoid killing normal cells. In certain embodiments, the antibodies of the application kill cancer cells and prevent their growth and/or migration.

In a particular embodiment, effector function of anti-CDCP1 antibodies is eliminated by swapping the IgG1 constant domain for an IgG2/4 fusion domain. Other ways of eliminating effector function can be envisioned such as, e.g., mutation of the sites known to interact with FcR or insertion of a peptide in the hinge region, thereby eliminating critical sites required for FcR interaction. Variant anti-CDCP1 antibodies with reduced or no effector function also include variants as described previously herein.

The anti-CDCP1 antibodies of the application may be used in combination with other therapies or with other agents. Other agents include but are not limited to polypeptides, small molecules, chemicals, metals, organometallic compounds, inorganic compounds, nucleic acid molecules, oligonucleotides, aptamers, spiegelmers, antisense nucleic acids, locked nucleic acid (LNA) inhibitors, peptide nucleic acid (PNA) inhibitors, immunomodulatory agents, antigen-binding fragments, prodrugs, and peptidomimetic compounds. In certain embodiments, the inhibitors of the application may be used in combination with prostate cancer therapies known to one of skill in the art including: surgery (radical prostatectomy), hormone therapy, radiotherapy and brachytherapy.

In certain aspects, the present disclosure relates to combination treatments comprising an anti-CDCP1 antibody including the antibodies described herein and immunomodulatory compounds, vaccines or chemotherapy. Illustrative examples of suitable immunomodulatory agents that may be used in such combination therapies include agents that block negative regulation of T cells or antigen presenting cells (e.g., anti-CTLA4 antibodies, anti-PD-L1 antibodies, anti-PDL-2 antibodies, anti-PD-1 antibodies and the like) or agents that enhance positive co-stimulation of T cells (e.g., anti-CD40 antibodies or anti-4-1BB antibodies) or agents that increase NK cell number or T-cell activity (e.g., inhibitors such as IMiDs, thalidomide, or thalidomide analogs). Furthermore, immunomodulatory therapy could include cancer vaccines such as dendritic cells loaded with tumor cells, proteins, peptides, RNA, or DNA derived from such cells, patient derived heat-shock proteins (hsp's) or general adjuvants stimulating the immune system at various levels such as CpG, Luivac®, Biostim®, Ribomunyl®, Imudon®, Bronchovaxom® or any other compound or other adjuvant activating receptors of the innate immune system (e.g., toll like receptor agonist, anti-CTLA-4 antibodies, etc.). Also, immunomodulatory therapy could include treatment with cytokines such as IL-2, GM-CSF and IFN-gamma.

Furthermore, combination of anti-CDCP1 therapy with chemotherapeutics could be particularly useful to reduce overall tumor burden, to limit angiogenesis, to enhance tumor accessibility, to enhance susceptibility to ADCC, to result in increased immune function by providing more tumor antigen, or to increase the expression of the T cell attractant LIGHT. When anti-CDCP1 therapy is administered to a subject in combination with another conventional anti-neoplastic agent, either concomitantly or sequentially, anti-CDCP1 therapy may be shown to enhance the therapeutic effect of either agent alone. Pharmaceutical compounds that may be used for combinatory anti-tumor therapy include, merely to illustrate: aminoglutethimide, amsacrine, anastrozole, asparaginase, bcg, bicalutamide, bleomycin, buserelin, busulfan, camptothecin, capecitabine, carboplatin, carmustine, chlorambucil, cisplatin, cladribine, clodronate, colchicine, cyclophosphamide, cyproterone, cytarabine, dacarbazine, dactinomycin, daunorubicin, dienestrol, diethylstilbestrol, docetaxel, doxorubicin, epirubicin, estradiol, estramustine, etoposide, exemestane, filgrastim, fludarabine, fludrocortisone, fluorouracil, fluoxymesterone, flutamide, gemcitabine, genistein, goserelin, hydroxyurea, idarubicin, ifosfamide, imatinib, interferon, irinotecan, letrozole, leucovorin, leuprolide, levamisole, lomustine, mechlorethamine, medroxyprogesterone, megestrol, melphalan, mercaptopurine, mesna, methotrexate, mitomycin, mitotane, mitoxantrone, nilutamide, nocodazole, octreotide, oxaliplatin, paclitaxel, pamidronate, pentostatin, plicamycin, porfimer, procarbazine, raltitrexed, rituximab, streptozocin, suramin, tamoxifen, temozolomide, teniposide, testosterone, thioguanine, thiotepa, titanocene dichloride, topotecan, trastuzumab, tretinoin, vinblastine, vincristine, vindesine, and vinorelbine.

These chemotherapeutic anti-tumor compounds may be categorized by their mechanism of action into groups, including, for example, the following classes of agents: anti-metabolites/anti-cancer agents, such as pyrimidine analogs (5-fluorouracil, floxuridine, capecitabine, gemcitabine and cytarabine) and purine analogs, folate inhibitors and related inhibitors (mercaptopurine, thioguanine, pentostatin and 2-chlorodeoxyadenosine (cladribine)); antiproliferative/antimitotic agents including natural products such as vinca alkaloids (vinblastine, vincristine, and vinorelbine), microtubule disruptors such as taxane (paclitaxel, docetaxel), vincristine, vinblastine, nocodazole, epothilones and navelbine, epidipodophyllotoxins (etoposide, teniposide), DNA damaging agents (actinomycin, amsacrine, anthracyclines, bleomycin, busulfan, camptothecin, carboplatin, chlorambucil, cisplatin, cyclophosphamide, cytoxan, dactinomycin, daunorubicin, doxorubicin, epirubicin, hexamethylmelamineoxaliplatin, iphosphamide, melphalan, mechlorethamine, mitomycin, mitoxantrone, nitrosourea, plicamycin, procarbazine, taxol, taxotere, teniposide, triethylenethiophosphoramide and etoposide (VP16)); antibiotics such as dactinomycin (actinomycin D), daunorubicin, doxorubicin (adriamycin), idarubicin, anthracyclines, mitoxantrone, bleomycins, plicamycin (mithramycin) and mitomycin; enzymes (L-asparaginase which systemically metabolizes L-asparagine and deprives cells which do not have the capacity to synthesize their own asparagine); antiplatelet agents; antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nitrosoureas (carmustine (BCNU) and analogs, streptozocin), trazenes-dacarbazinine (DTIC); antiproliferative/antimitotic antimetabolites such as folic acid analogs (methotrexate); platinum coordination complexes (cisplatin, carboplatin), procarbazine, hydroxyurea, mitotane, aminoglutethimide; hormones, hormone analogs (estrogen, tamoxifen, goserelin, bicalutamide, nilutamide) and aromatase inhibitors (letrozole, anastrozole); anticoagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory agents; antisecretory agents (breveldin); immunosuppressives (cyclosporine, tacrolimus (FK-506), sirolimus (rapamycin), azathioprine, mycophenolate mofetil); immunomodulatory agents (thalidomide and analogs thereof such as lenalidomide (Revlimid, CC-5013) and CC-4047 (Actimid)), cyclophosphamide; anti-angiogenic compounds (TNP-470, genistein) and growth factor inhibitors (vascular endothelial growth factor (VEGF) inhibitors, fibroblast growth factor (FGF) inhibitors); angiotensin receptor blocker; nitric oxide donors; anti-sense oligonucleotides; antibodies (trastuzumab); cell cycle inhibitors and differentiation inducers (tretinoin); mTOR inhibitors, topoisomerase inhibitors (doxorubicin (adriamycin), amsacrine, camptothecin, daunorubicin, dactinomycin, eniposide, epirubicin, etoposide, idarubicin and mitoxantrone, topotecan, irinotecan), corticosteroids (cortisone, dexamethasone, hydrocortisone, methylprednisolone, prednisone, and prenisolone); growth factor signal transduction kinase inhibitors; mitochondrial dysfunction inducers and caspase activators; and chromatin disruptors.

In certain embodiments, pharmaceutical compounds that may be used for combinatory anti-angiogenesis therapy include: (1) inhibitors of release of “angiogenic molecules,” such as bFGF (basic fibroblast growth factor); (2) neutralizers of angiogenic molecules, such as anti-βbFGF antibodies; and (3) inhibitors of endothelial cell response to angiogenic stimuli, including collagenase inhibitor, basement membrane turnover inhibitors, angiostatic steroids, fungal-derived angiogenesis inhibitors, platelet factor 4, thrombospondin, arthritis drugs such as D-penicillamine and gold thiomalate, vitamin D₃ analogs, alpha-interferon, and the like. For additional proposed inhibitors of angiogenesis, see Blood et al., Biochim. Biophys. Acta, 1032:89-118 (1990), Moses et al., Science, 248:1408-1410 (1990), Ingber et al., Lab. Invest., 59:44-51 (1988), and U.S. Pat. Nos. 5,092,885, 5,112,946, 5,192,744, 5,202,352, and 6,573,256. In addition, there are a wide variety of compounds that can be used to inhibit angiogenesis, for example, peptides or agents that block the VEGF-mediated angiogenesis pathway, endostatin protein or derivatives, lysine binding fragments of angiostatin, melanin or melanin-promoting compounds, plasminogen fragments (e.g., Kringles 1-3 of plasminogen), troponin subunits, inhibitors of vitronectin αᵥβ₃, peptides derived from Saposin B, antibiotics or analogs (e.g., tetracycline or neomycin), dienogest-containing compositions, compounds comprising a MetAP-2 inhibitory core coupled to a peptide, the compound EM-138, chalcone and its analogs, and naaladase inhibitors. See, for example, U.S. Pat. Nos. 6,395,718, 6,462,075, 6,465,431, 6,475,784, 6,482,802, 6,482,810, 6,500,431, 6,500,924, 6,518,298, 6,521,439, 6,525,019, 6,538,103, 6,544,758, 6,544,947, 6,548,477, 6,559,126, and 6,569,845.

Depending on the nature of the combinatory therapy, administration of the anti-CDCP1 antibody may be continued while the other therapy is being administered and/or thereafter. Administration of the antibody may be made in a single dose, or in multiple doses. In some instances, administration of the anti-CDCP1 antibody is commenced at least several days prior to the conventional therapy, while in other instances, administration is begun either immediately before or at the time of the administration of the conventional therapy. In some cases, the anti-CDCP1 antibody will be administered after other therapies, or it could be administered alternating with other therapies.

In certain embodiments, the antibodies of the application may bind to a functional domain of CDCP1. In certain embodiments, the antibodies of the application may bind to a CUB domain of CDCP1. In certain embodiments, the antibodies of the application may bind to the regions of CDCP1 that bind the Src SH2 domain, N-cadherin, P-cadherin, syndecan 1, syndecan 4, MT-SP1, or the C2 domain of PKCδ. In certain embodiments, the antibodies of the application may block the interaction between CDCP1 and N-cadherin, P-cadherin, syndecan 1, syndecan 4, or MT-SP1.

In yet another embodiment, the cancer treatment involves administering an antibody that (1) is conjugated to a cytotoxic agent, (2) blocks the interaction between CDCP1 and the Src SH2 domain, N-cadherin, P-cadherin, syndecan 1, syndecan 4, MT-SP1, or the C2 domain of PKCδ and (3) attracts T cells to the tumor cells. T cell attraction can be achieved by fusing the Ab with chemokines such as MIG, IP-10, I-TAC, CCL21, CCL5 or LIGHT. Also, treatment with chemotherapeutics can result in the desired upregulation of LIGHT. The combined action of blocking immune suppression and killing directly through antibody targeting of the tumor cells is a unique approach that provides increased efficacy.

In certain embodiments, the application is directed to a method of modulating at least one biological activity of CDCP1 in a subject in need thereof comprising administering to said subject an effective amount of an anti-CDCP1 antibody.

The present application includes a method of inhibiting the proliferation or anchorage-independent growth of cancer cells comprising contacting cancer cells with an anti-CDCP1 antibody. The antibodies may contact cancer cells in vitro, ex vivo or in vivo (for example, in a subject). In one embodiment, the antibodies are anti-CDCP1 antibodies including the antibodies, antibody fragments, and antibody conjugates of the present application. Such a modulation reduces the cancer cell proliferation or anchorage independent growth by at least 10%, at least 25%, at least 50%, at least 75%, or at least 90%.

The present application provides for a method of inhibiting the growth of cancer cells in a subject comprising administering an effective amount of an anti-CDCP1 antibody into the subject. The modulation may reduce or prevent the growth of the cancer cells of said subject, such as for example, by at least 10%, at least 25%, at least 50%, at least 75%, or at least 90%. As a result, where the cancer is a solid tumor, the modulation may reduce the size of the solid tumor by at least 10%, at least 25%, at least 50%, at least 75%, or at least 90%.

The inhibition of the cancer cell proliferation can be measured by cell-based assays, such as bromodeoxyuridine (BRDU) incorporation (Hoshino et al., Int. J. Cancer 38, 369 (1986); Campana et al., J. Immunol. Meth. 107:79 (1988)); [³H]-thymidine incorporation (Chen, J., Oncogene 13:1395-403 (1996); Jeoung, J., J. Biol. Chem. 270:18367-73 (1995); the dye Alamar Blue (available from Biosource International) (Voytik-Harbin et al., In Vitro Cell Dev Biol Anim 34:239-46 (1998)). The anchorage independent growth of cancer cells is assessed by colony formation assay in soft agar, such as by counting the number of cancer cell colonies formed on top of the soft agar (see Examples and Sambrook et al., Molecular Cloning, Cold Spring Harbor, 1989).

The inhibition of cancer cell growth in a subject may be assessed by monitoring the cancer growth in a subject, for example in an animal model or in human patients. One exemplary monitoring method is tumorigenicity assays. In one example, a xenograft comprises human cells from a pre-existing tumor or from a tumor cell line. Tumor xenograft assays are known in the art and described herein (see, e.g., Ogawa et al., Oncogene 19:6043-6052 (2000)). In another embodiment, tumorigenicity is monitored using the hollow fiber assay, which is described in U.S. Pat. No. 5,698,413, which is incorporated herein by reference in its entirety.

The percentage of the inhibition is calculated by comparing the cancer cell proliferation, anchorage independent growth, or cancer cell growth under antibody treatment with that under negative control condition (typically without antibody treatment). For example, where the number of cancer cells or cancer cell colonies (colony formation assay), or BRDU or [³H]-thymidine incorporation is A (under the treatment of antibodies) and C (under negative control condition), the percentage of inhibition would be (C−A)/C×100%.

Angiogenesis, the formation of new capillaries from pre-existing vessels, is essential for tumor progression (Folkman, et al., J. Biol. Chem. 267:10931-10934 (1992)). The induction of angiogenesis is mediated by several angiogenic molecules released by tumor cells, tumor associated endothelial cells and the normal cells surrounding the tumor endothelial cells. The prevascular stage of a tumor is associated with local benign tumors, whereas the vascular stage is associated with tumors capable of metastasizing. Moreover, studies using light microscopy and immunohistochemistry concluded that the number and density of microvessels in different human cancers directly correlate with their potential to invade and produce metastasis. The inhibition of angiogenesis prevents the growth of tumor endothelial cells at both the primary and secondary sites and thus can prevent the emergence of metastases.

Both controlled and uncontrolled angiogenesis are thought to proceed in a similar manner. Endothelial cells and pericytes, surrounded by a basement membrane, form capillary blood vessels. Angiogenesis begins with the erosion of the basement membrane by enzymes released by endothelial cells and leukocytes. The endothelial cells, which line the lumen of blood vessels, then protrude through the basement membrane. Angiogenic stimulants induce the endothelial cells to migrate through the eroded basement membrane. The migrating cells form a “sprout” off the parent blood vessel, where the endothelial cells undergo mitosis and proliferate. The endothelial sprouts merge with each other to form capillary loops, creating the new blood vessel.

The present application provides for a method of inhibiting angiogenesis comprising contacting endothelial cells with an effective amount of an anti-CDCP1 antibody. In certain embodiments, said angiogenesis is induced by cancer cells. The antibodies may contact endothelial cells in vitro, ex vivo or in vivo (for example, in a subject). In still another embodiment, the antibodies inhibit the angiogenesis of cancer cells, such as for example, by at least 10%, 25%, 50%, 75%, or 90%. In one example, the cancer cells are cells of prostate cancer.

In one embodiment, the present application provides for a method of inhibiting angiogenesis in a subject comprising administering an effective amount of an anti-CDCP1 antibody described herein to said subject.

In another embodiment, the present application provides for a method of inhibiting metastasis of cancer in a subject comprising administering an effective amount of an anti-CDCP1 antibody described herein to said subject. In still another embodiment, the antibodies inhibit the metastasis of cancer cells, such as for example, by at least 10%, 25%, 50%, 75%, or 90%. In one example, the cancer cells are cells of prostate cancer.

The inhibition of angiogenesis can be examined via in vitro cell-based assays known in the art, such as the tube formation assay, or in vivo animal model assays known in the art. The inhibition of metastasis can be assessed in an in vivo animal metastasis model.

In addition to the inhibition of cancer growth, angiogenesis, and cancer cell metastasis, the anti-CDCP1 antibodies of the present application may also be able to inhibit adhesion, migration or invasion of cancer cells via contacting the cancer cells with the anti-CDCP1 antibodies of the present application.

The antibody may also inhibit the survival of cancer cells or induce cancer cell apoptosis. Cancer cell survival can be assessed by counting the number of living cancer cells. Induction of apoptosis can be measured by the various ways known in the art, such as by flow cytometry with FITC-conjugated annexin V and propidium iodide or terminal deoxynucleotidyl transferase-mediated digoxigenin-11-dUTP nick end labeling (TUNEL) assay (Lazebnik et al., Nature 371:346 (1994) and Yonehara et al., J. Exp. Med. 169:1747 (1989)).

The anti-CDCP1 antibodies of the present application may be of a subclass or isotype that is capable of mediating the cytolysis of tumor cells via antibody dependent cellular cytotoxicity (ADCC) and therefore lead to tumor cell killing. The antibodies may be of subclass IgG3, IgG2a or IgG2b where the antibodies are mouse immunoglobulins, and IgG1 where the antibodies are human immunoglobulins.

In certain embodiments, treatment of prostate cancer according to the present application may be combined with other treatment methods known in the art (i.e., combination therapy), such as, radical or salvage prostatectomy, external beam irradiation therapy, interstitial seed implantation (brachytherapy), hormonal therapy and androgen ablation and chemotherapy (for additional information on treatment options available to date see http://psa-rising.com/caplinks/medical_{—}txmodes.htm). In certain embodiments, treatment of other cancer types may be combined with cancer type specific treatment methods known in the art.

Methods of administration of therapeutic agents, particularly antibody therapeutics, are well-known to those of skill in the art. The pharmaceutical formulations, dosage forms, and uses described below generally apply to antibody-based therapeutic agents, but are also useful and can be modified, where necessary, for making and using therapeutic agents of the disclosure that are not antibodies.

To achieve the desired therapeutic effect, the anti-CDCP1 antibodies or antigen-binding fragments thereof can be administered in a variety of unit dosage forms. The dose will vary according to the particular antibody. For example, different antibodies may have different masses and/or affinities, and thus require different dosage levels. Antibodies prepared as Fab or other fragments will also require differing dosages than the equivalent intact immunoglobulins, as they are of considerably smaller mass than intact immunoglobulins, and thus require lower dosages to reach the same molar levels in the patient's blood. The dose will also vary depending on the manner of administration, the particular symptoms of the patient being treated, the overall health, condition, size, and age of the patient, and the judgment of the prescribing physician. Dosage levels of the antibodies for human subjects are generally between about 1 mg per kg and about 100 mg per kg per patient per treatment, such as for example, between about 5 mg per kg and about 50 mg per kg per patient per treatment. In terms of plasma concentrations, the antibody concentrations may be in the range from about 25 μg/mL to about 500 μg/mL. However, greater amounts may be required for extreme cases and smaller amounts may be sufficient for milder cases.

Administration of the anti-CDCP1 antibodies will generally be performed by an intravascular route, e.g., via intravenous infusion by injection. Other routes of administration may be used if desired but an intravenous route will be the most preferable. Formulations suitable for injection are found in Remington's Pharmaceutical Sciences, Mack Publishing Company, Philadelphia, Pa., 17th ed. (1985). Such formulations must be sterile and non-pyrogenic, and generally will include a pharmaceutically effective carrier, such as saline, buffered (e.g., phosphate buffered) saline, Hank's solution, Ringer's solution, dextrose/saline, glucose solutions, and the like. The formulations may contain pharmaceutically acceptable auxiliary substances as required, such as, tonicity adjusting agents, wetting agents, bactericidal agents, preservatives, stabilizers, and the like.

Administration of an anti-CDCP1 antibody will generally be performed by a parenteral route, typically via injection such as intra-articular or intravascular injection (e.g., intravenous infusion) or intramuscular injection. Other routes of administration, e.g., oral (p.o.), may be used if desired and practicable for the particular antibody to be administered. Anti-CDCP1 antibody can also be administered in a variety of unit dosage forms and their dosages will also vary with the size, potency, and in vivo half-life of the particular antibody being administered. Doses of an anti-CDCP1 antibody will also vary depending on the manner of administration, the particular symptoms of the patient being treated, the overall health, condition, size, and age of the patient, and the judgment of the prescribing physician.

In certain embodiments, a typical therapeutic treatment includes a series of doses, which will usually be administered concurrently with the monitoring of clinical endpoints of prostate cancer such as clinical stage, Gleason scores, tumor antigen levels, tumor size, and pathologic state, etc., with the dosage levels adjusted as needed to achieve the desired clinical outcome. In certain embodiments, treatment is administered in multiple dosages over at least a week. In certain embodiments, treatment is administered in multiple dosages over at least a month. In certain embodiments, treatment is administered in multiple dosages over at least a year. In certain embodiments, treatment is administered in multiple dosages over the remainder of the patient's life. In certain embodiments, treatment is administered chronically. “Chronically” as used herein, is meant to refer to administering the therapeutic for a period of at least 3 months, such as for example, a period of at least 1 year, or for the duration of the disease in the patient.

The frequency of administration may also be adjusted according to various parameters. These include the clinical response, the plasma half-life of the antibody, and the levels of the antibody in a body fluid, such as, blood, plasma, serum, or synovial fluid. To guide adjustment of the frequency of administration, levels of the antibody in the body fluid may be monitored during the course of treatment.

For the treatment of prostate cancer by systemic administration of an anti-CDCP1 antibody (as opposed to local administration), administration of a large initial dose is specific, i.e., a single initial dose sufficient to yield a substantial reduction, such as for example, at least about 50% reduction in CDCP1 activity. Such a large initial dose may be followed by regularly repeated administration of tapered doses as needed to maintain substantial reductions in CDCP1 activity. In another embodiment, the initial dose is given by both local and systemic routes, followed by repeated systemic administration of tapered doses as described above.

Formulations particularly useful for antibody-based therapeutic agents are also described in U.S. Patent App. Publication Nos. 20030202972, 20040091490 and 20050158316. In certain embodiments, the liquid formulations of the application are substantially free of surfactant and/or inorganic salts. In another specific embodiment, the liquid formulations have a pH ranging from about 5.0 to about 7.0. In yet another specific embodiment, the liquid formulations comprise histidine at a concentration ranging from about 1 mM to about 100 mM. In still another specific embodiment, the liquid formulations comprise histidine at a concentration ranging from 1 mM to 100 mM. It is also contemplated that the liquid formulations may further comprise one or more excipients such as a saccharide, an amino acid (e.g., arginine, lysine, and methionine) and a polyol. Additional descriptions and methods of preparing and analyzing liquid formulations can be found, for example, in PCT publications WO 03/106644, WO 04/066957, and WO 04/091658.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the pharmaceutical compositions of the application.

In certain embodiments, formulations of the subject antibodies are pyrogen-free formulations which are substantially free of endotoxins and/or related pyrogenic substances. Endotoxins include toxins that are confined inside microorganisms and are released when the microorganisms are broken down or die. Pyrogenic substances also include fever-inducing, thermostable substances (glycoproteins) from the outer membrane of bacteria and other microorganisms. Both of these substances can cause fever, hypotension and shock if administered to humans. Due to the potential harmful effects, it is advantageous to remove even low amounts of endotoxins from intravenously administered pharmaceutical drug solutions. The Food & Drug Administration (“FDA”) has set an upper limit of 5 endotoxin units (EU) per dose per kilogram body weight in a single one hour period for intravenous drug applications (The United States Pharmacopeial Convention, Pharmacopeial Forum 26 (1):223 (2000)). When therapeutic proteins are administered in amounts of several hundred or thousand milligrams per kilogram body weight, as can be the case with monoclonal antibodies, it is advantageous to remove even trace amounts of endotoxin.

Formulations of the subject antibodies include those suitable for oral, dietary, topical, parenteral (e.g., intravenous, intraarterial, intramuscular, subcutaneous injection), opthalmologic (e.g., topical or intraocular), inhalation (e.g., intrabronchial, intranasal or oral inhalation, intranasal drops), rectal, and/or intravaginal administration. Other suitable methods of administration can also include rechargeable or biodegradable devices and controlled release polymeric devices. Stents, in particular, may be coated with a controlled release polymer mixed with an agent of the application. The pharmaceutical compositions of this disclosure can also be administered as part of a combinatorial therapy with other agents (either in the same formulation or in a separate formulation).

The amount of the formulation which will be therapeutically effective can be determined by standard clinical techniques. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each patient's circumstances. Effective doses may be extrapolated from dose-response curves derived from in vitro or animal model test systems. The dosage of the compositions to be administered can be determined by the skilled artisan without undue experimentation in conjunction with standard dose-response studies. Relevant circumstances to be considered in making those determinations include the condition or conditions to be treated, the choice of composition to be administered, the age, weight, and response of the individual patient, and the severity of the patient's symptoms. For example, the actual patient body weight may be used to calculate the dose of the formulations in milliliters (mL) to be administered. There may be no downward adjustment to “ideal” weight. In such a situation, an appropriate dose may be calculated by the following formula: Dose (mL)=[patient weight (kg)×dose level (mg/kg)/drug concentration (mg/mL)]

To achieve the desired reductions of body fluid parameters, such anti-CDCP1 antibodies can be administered in a variety of unit dosage forms. The dose will vary according to the particular antibody. For example, different antibodies may have different masses and/or affinities, and thus require different dosage levels. Antibodies prepared as Fab′ fragments or single chain antibodies will also require differing dosages than the equivalent native immunoglobulins, as they are of considerably smaller mass than native immunoglobulins, and thus require lower dosages to reach the same molar levels in the patient's blood.

Other therapeutics of the disclosure can also be administered in a variety of unit dosage forms and their dosages will also vary with the size, potency, and in vivo half-life of the particular therapeutic being administered.

For the purpose of treatment of disease, the appropriate dosage of the compounds (for example, antibodies) will depend on the severity and course of disease, the patient's clinical history and response, the toxicity of the antibodies, and the discretion of the attending physician. The initial candidate dosage may be administered to a patient. The proper dosage and treatment regimen can be established by monitoring the progress of therapy using conventional techniques known to those of skill in the art.

The formulations of the application can be distributed as articles of manufacture comprising packaging material and a pharmaceutical agent which comprises, e.g., the antibody and a pharmaceutically acceptable carrier as appropriate to the mode of administration. The packaging material will include a label which indicates that the formulation is for use in the treatment of prostate cancer.

In a further embodiment, recombinant DNA including an insert coding for a heavy chain variable domain and/or for a light chain variable domain of cancer-binding antibodies described hereinbefore are produced. The term DNA includes coding single stranded DNAs, double stranded DNAs consisting of said coding DNAs and of complementary DNAs thereto, or these complementary (single stranded) DNAs themselves. Furthermore, DNA encoding a heavy chain variable domain and/or a light chain variable domain of the cancer-binding antibodies disclosed herein can be enzymatically or chemically synthesized DNA having the authentic DNA sequence coding for a heavy chain variable domain and/or for the light chain variable domain, or a mutant thereof. A mutant of the authentic DNA is a DNA encoding a heavy chain variable domain and/or a light chain variable domain of the above-mentioned antibodies in which one or more amino acids are deleted or exchanged with one or more other amino acids. Preferably said modification(s) are outside the CDRs of the heavy chain variable domain and/or of the light chain variable domain of the antibody in humanization and expression optimization applications. The term mutant DNA also embraces silent mutants wherein one or more nucleotides are replaced by other nucleotides with the new codons coding for the same amino acid(s). The term mutant sequence also includes a degenerate sequence. Degenerate sequences are degenerate within the meaning of the genetic code in that an unlimited number of nucleotides are replaced by other nucleotides without resulting in a change of the amino acid sequence originally encoded. Such degenerate sequences may be useful due to their different restriction sites and/or frequency of particular codons which are preferred by the specific host, particularly E. coli, to obtain an optimal expression of the heavy chain murine variable domain and/or a light chain murine variable domain.

The term mutant is intended to include a DNA mutant obtained by in vitro mutagenesis of the authentic DNA according to methods known in the art.

For the assembly of complete tetrameric immunoglobulin molecules and the expression of chimeric antibodies, the recombinant DNA inserts coding for heavy and light chain variable domains are fused with the corresponding DNAs coding for heavy and light chain constant domains, then transferred into appropriate host cells, for example after incorporation into hybrid vectors.

Recombinant DNAs including an insert coding for a heavy chain murine variable domain of an antibody directed to the cell line disclosed herein fused to a human IgG heavy chain constant domain, for example γ1, γ2, γ3 or γ4, preferably γ1 or γ4 are also provided. Recombinant DNAs including an insert coding for a light chain murine variable domain of an antibody directed to the cell line disclosed herein fused to a human constant domain K or x, preferably K, are also provided

Another embodiment pertains to recombinant DNAs coding for a recombinant polypeptide wherein the heavy chain variable domain and the light chain variable domain are linked by way of a spacer group, optionally including a signal sequence facilitating the processing of the antibody in the host cell and/or a DNA coding for a peptide facilitating the purification of the antibody and/or a cleavage site and/or a peptide spacer and/or an effector molecule. The DNA coding for an effector molecule is intended to be a DNA coding for the effector molecules useful in diagnostic or therapeutic applications. Thus, effector molecules which are toxins or enzymes, especially enzymes capable of catalyzing the activation of prodrugs, are particularly indicated. The DNA encoding such an effector molecule has the sequence of a naturally occurring enzyme or toxin encoding DNA, or a mutant thereof, and can be prepared by methods well known in the art.

In certain embodiments, the antibodies of the application may further comprise an additional prostate cancer targeting agent. In certain embodiments, the agent targets any prostate tissue. In certain embodiments, the targeting agent is a peptide that specifically binds to prostate cancer cells such as those described in US Patent Application Nos. 20060239968 and 20010046498 incorporated by reference in their entirety herein. In certain embodiments, the peptides may be fused to the antibodies of the application. In certain embodiments, the targeting agent is an aptamer that specifically binds to prostate cancer cells such as those described in Farokhzad et al., Proc. Natl. Acad. Sci. U.S.A. April 18; 103(16):6315-20 (2006) incorporated by reference in their entirety herein.

In order that those skilled in the art may be better able to practice the compositions and methods described herein, the following examples are given for illustration purposes.

### EXAMPLE 1

### Cross-Reactivity of Antisera Derived from Human Cancer Cell Line Immunized Animals to RBCs and WBCs

Individual mice were immunized with one each of eight cancer cell lines, representing six different types of solid tumors. To evaluate murine immune response, binding of each antiserum (pre- and post-bleed) to the immunizing cancer cell line was compared to binding to human RBCs and WBCs by flow cytometry. As expected, antisera showed strong binding to the immunizing cancer cells (Table 1). However, these antisera also showed very strong binding to human RBCs and WBCs. In all cell lines (8/8) the binding of the antisera to RBCs is stronger than binding to the immunizing cancer cells. In half of the cell lines (4/8), binding of antisera to WBCs is stronger than binding to cancer cells.

TABLE 1
Flow cytometric analysis of binding of anticancer sera to cancer cells and human blood cells. The average from
all animals per cell line is depicted in terms of geomean fluorescence intensity of post-bleed divided by pre-bleed.
Flow cytometric analyses were done with 500,000 cells per reaction, with serum dilution factor of 100.

| | Cancer | Number of | Binding to Cancer Cells | Cross-Reactivitv to RBC | Cross-Reactivitv to WBC |
|---|---|---|---|---|---|
| Cell Lines | Type | animals | Post-bleed/Pre-bleed* | Post-bleed/Pre-bleed* | Post-bleed/Pre-bleed* |
| MDA-MB-435 | Breast | 5 | 350 | 1195 | 575 |
| MCF-7 | Breast | 3 | 300 | 576 | 487 |
| SK-OV3 | Ovary | 5 | 178 | 852 | 450 |
| PC-3 | Prostate | 3 | 400 | 1300 | 642 |
| Dul45 | Prostate | 5 | 420 | 1108 | 185 |
| KM12L4a | Colon | 5 | 300 | 668 | 238 |
| A-431 | Head & Neck | 3 | 275 | 526 | 233 |
| Caki-1 | Renal | 4 | 310 | 907 | 181 |

### EXAMPLE 2

### Binding of Antisera to RBCs, WBCs and Cancer Cells after Blood Cell Subtraction

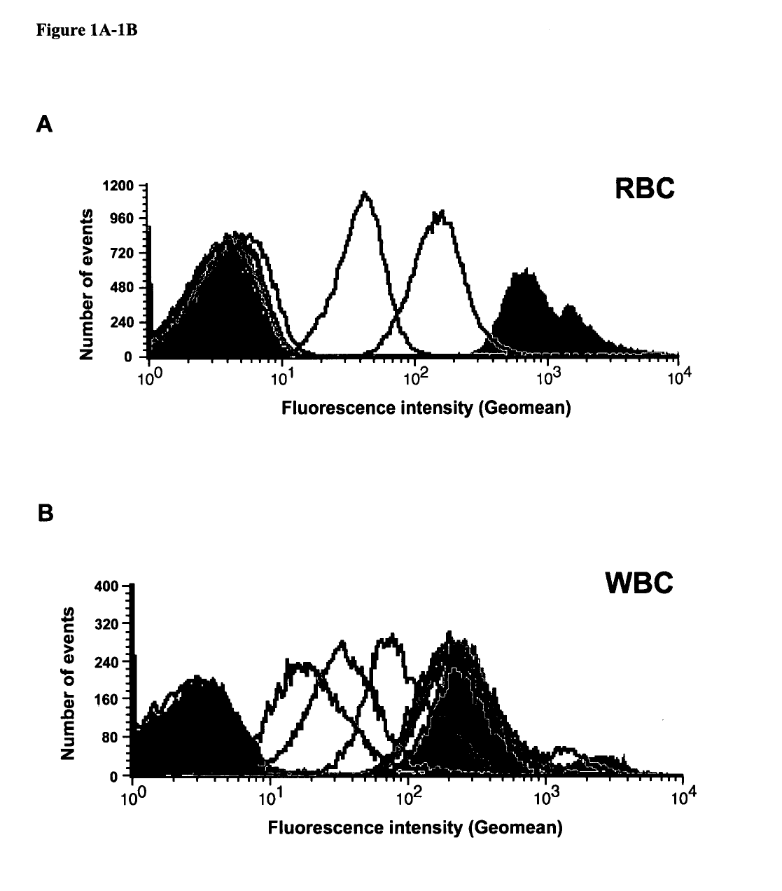

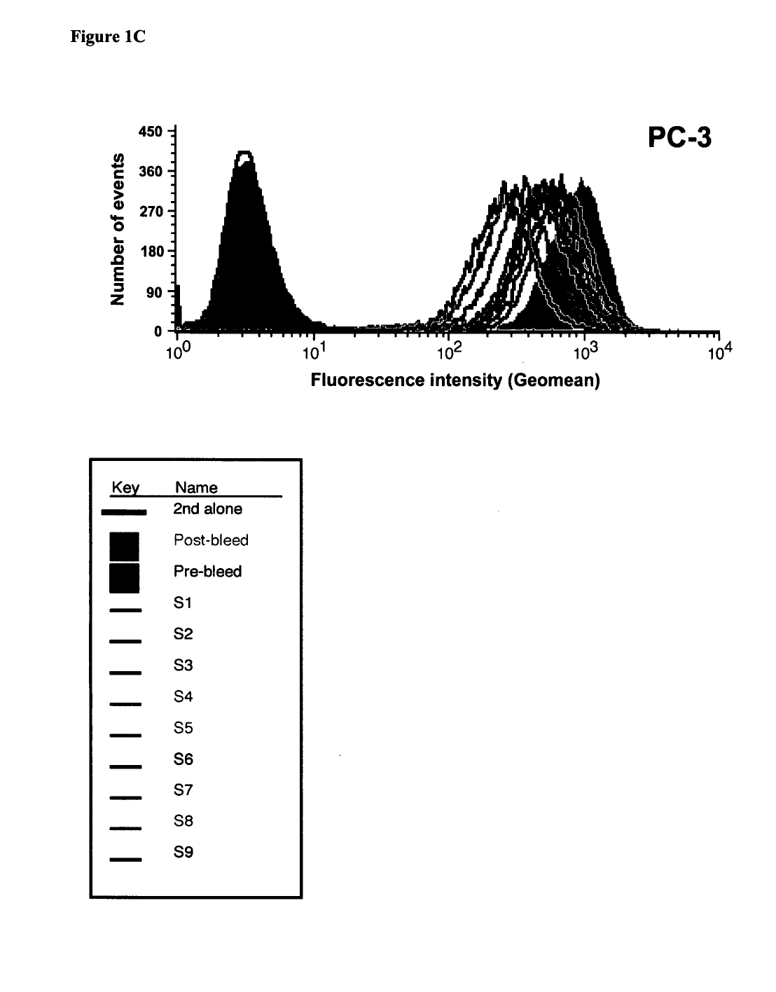

To determine whether it would be possible to select for tumor specificity by quantitatively depleting antibodies that bind to RBCs and WBCs, one PC-3-immunized mouse was randomly chosen for serum subtraction. PC-3 antiserum was subtracted with RBCs six times (Subtraction S1 to S6) and WBCs three times (S7 to S9) as described in the methods. Prior to subtraction, the PC-3 post-bleed antiserum from the selected mouse had about 250-fold higher binding to human RBCs than the pre-bleed antiserum. After four rounds of RBC subtraction, PC-3 antiserum binding to RBCs was completely depleted (FIG. 1A). Similarly, after three additional rounds of WBC subtraction, the binding of the subtracted PC-3 anti-serum to WBCs decreased from 112-fold (post-bleed) to only 7-fold higher than the pre-bleed (FIG. 1B).

Although subtraction of the antiserum with RBCs and WBCs was effective in decreasing undesirable binding, there was concern that the antibodies binding to PC-3 cells would be depleted as well. Prior to subtraction, the PC-3 post-bleed antisera demonstrated PC-3 binding at about 200-fold higher than the pre-bleed. After six rounds of RBC subtraction and three rounds of WBC subtraction, antiserum binding to PC-3 cells was reduced to about 60-fold higher than the pre-bleed, demonstrating that antibodies with specificity for PC-3 cells remained (FIG. 1C).

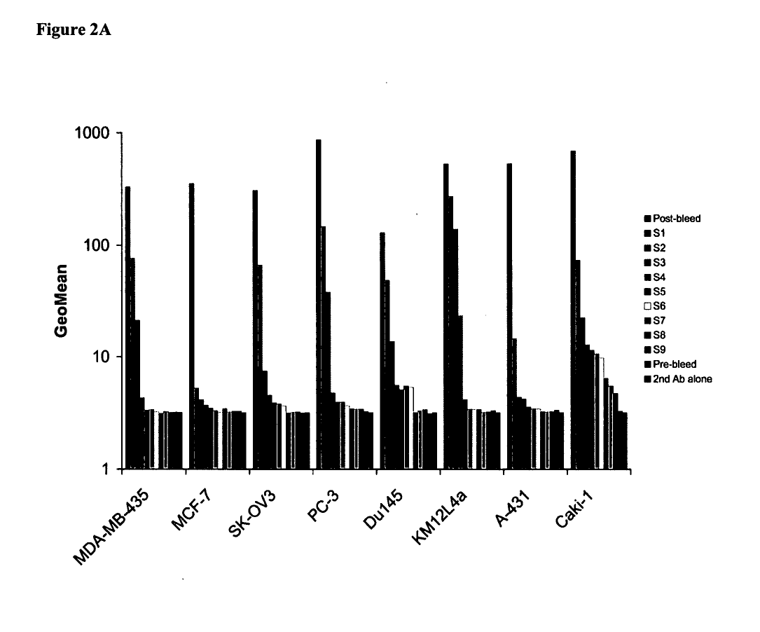

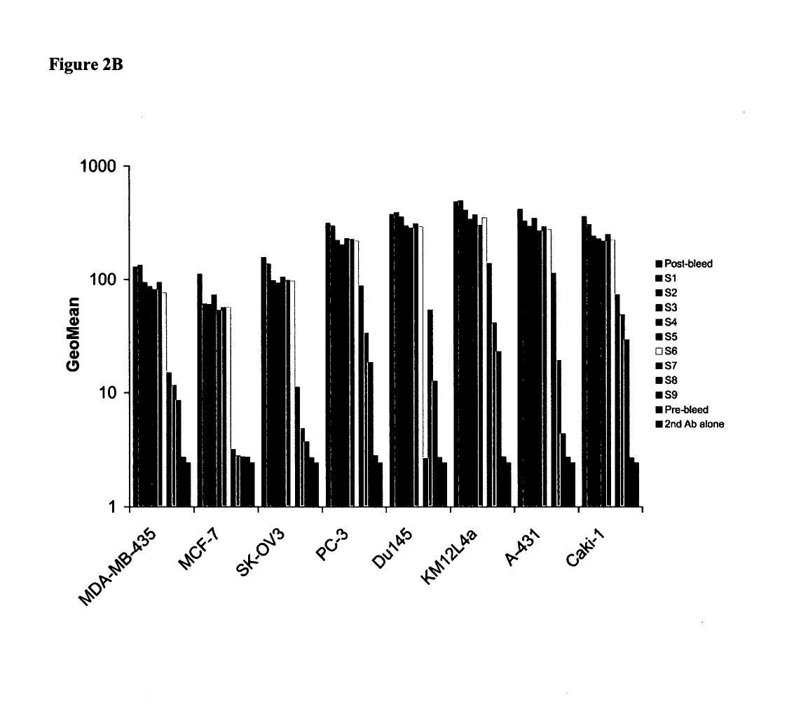

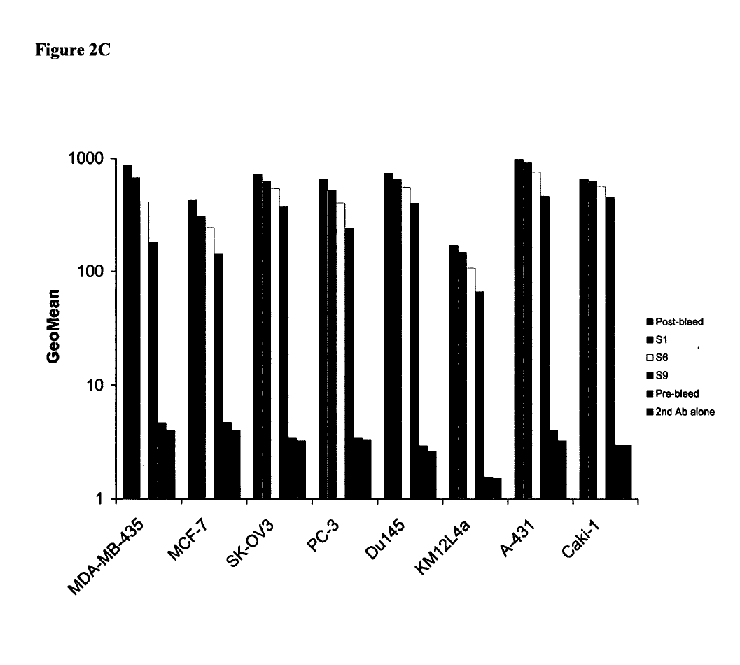

Additional sera were similarly examined by random selection of one serum sample per cancer cell line-immunized mouse. Serum samples from the pre-bleed and post-bleed time points, along with the sera following each round of RBC subtraction, were assayed for RBC binding (FIG. 2A). Stringent RBC subtraction for six rounds completely depleted antibodies against RBC epitopes in antisera from all cancer cell line-immunized animals, with the exception of Caki-1, which showed considerable depletion by the sixth round.

After three rounds of WBC subtraction, antibodies against the WBC epitopes were completely depleted from antisera from MCF-7, SK-OV3 and A-431 (FIG. 2B). Antisera from MDA-MB-435, PC-3, Du145, KM12L4a and Caki-1 cell lines showed considerable reduction (2- to 10-fold decrease) in WBC binding but not complete depletion. Since the RBC subtraction was performed with approximately 10⁹ cells, but the WBC subtraction was performed with only about 10⁷ cells, the incomplete subtraction of WBC-binding antibodies suggests that ideally the number of negative absorber cells needs to be at least in the 10⁹ range for complete depletion of sera (although it may be challenging to obtain such large numbers of WBCs or other normal tissues). Despite such stringent subtraction, specific binding of the blood cell-subtracted antisera to immunizing cancer cells was retained in all cases (FIG. 2C). After all rounds of subtraction, SK-OV3, Du145 and Caki-1 antisera retained the strongest binding to cancer cells (>50% of the post-bleed); antisera against MCF-7, KM12L4a and A-431 retained fairly strong binding (30% to 50% of the post-bleed); and antisera against MDA-MB-435 and PC-3 lost the most binding intensity to cancer cells (<30% of the post-bleed). These results suggest that the MDA-MB-435 breast cancer and PC-3 prostate cancer cell lines may share more immunogenic or abundant surface antigens with human blood cells than other cancer cell lines examined.

The polyclonal antibodies remaining after the negative selection process can be used as a therapeutic in treating cancer patients.

### EXAMPLE 3

### Comparison of PC-3 Whole Cell Panning with and without RBC Subtraction

In order to extend the results obtained using stringent negative selection of antisera on blood cells to a phage-displayed antibody library, we built a combinatorial antibody library from a PC-3 immunized mouse and compared the outcomes of whole-cell panning processes done with two methods of normal prostate cell (PrEC) subtraction, either with or without RBC subtraction (Table 2).

TABLE 2
Selection Parameters and Screening Results Following
Panning Rounds with or without RBC subtraction

| | | | | Ratio of | | |
|---|---|---|---|---|---|---|
| | | | Cell | phage input | % PrEC (−) of | % RBC (−) of |
| Pan | Round (R) | Cells | number | to cell | PC-3 binders | PC-3 binders |
| Without | R1 | PC-3 | 7.5 × 10⁶ | 1.4 × 10⁶ | ND | ND |
| RBC | Subtraction (12 h × 2) | PrEC | 8.0 × 10⁶ | 2.5 × 10⁵ | NA | NA |
| Subtraction | R2 | PC-3 | 7.5 × 10⁶ | 1.3 × 10⁵ | 60.0 | ND |
| | R3 | PC-3 | 3.8 × 10⁶ | 2.6 × 10⁶ | 34.0 | 0 |
| With RBC | R1 | PC-3 | 7.5 × 10⁶ | 1.4 × 10⁶ | ND | ND |
| Subtraction | Subtraction (1 h × 3) | RBC | 5.4 × 10⁹ | 700 | NA | NA |
| | Subtraction (2 h × 2) | PrEC | 3.75 × 10⁶ | 3.2 × 10⁵ | NA | NA |
| | R2 | PC-3 | 7.5 × 10⁶ | 2.2 × 10⁵ | 25.0 | 25.0 |
| | R3 | PC-3 | 7.5 × 10⁶ | 7.3 × 10⁵ | 9.6 | 16.4 |

* NA: Not applicable
** ND: Not determined

We evaluated two panning methods similar to traditional subtraction on normal counterpart cells with a phage to absorber cell ratio in the range of 10⁵-10⁶ to 1. In the panning where subtraction on PrEC was performed twice for 12 h each time without RBC subtraction, 34% of the final PC-3 binders from Round 3 were PrEC negative. In panning with PrEC subtraction twice for 2 h with RBC subtraction, 9.6% of the PC-3 binders from Round 3 were PrEC negative.

RBC subtraction was evaluated in the panning at a phage to absorber cell ratio of 700 to 1. In panning without RBC subtraction, none of the final antibodies identified from Round 3 were found to be RBC negative. However, when RBC subtraction was incorporated into the panning process, 16.4% of the final PC-3 binders did not bind to RBCs.

### EXAMPLE 4

### Analysis of Antibody and Target Diversity

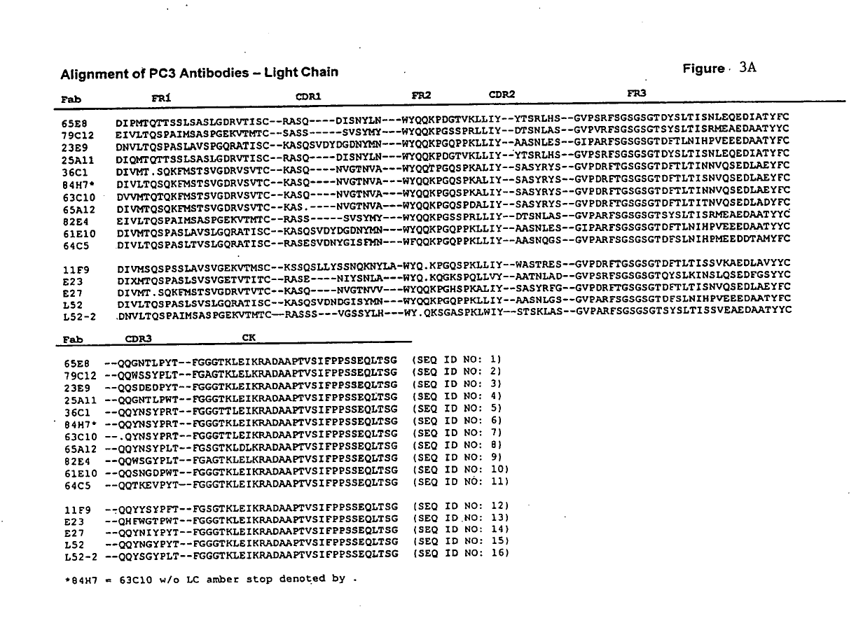

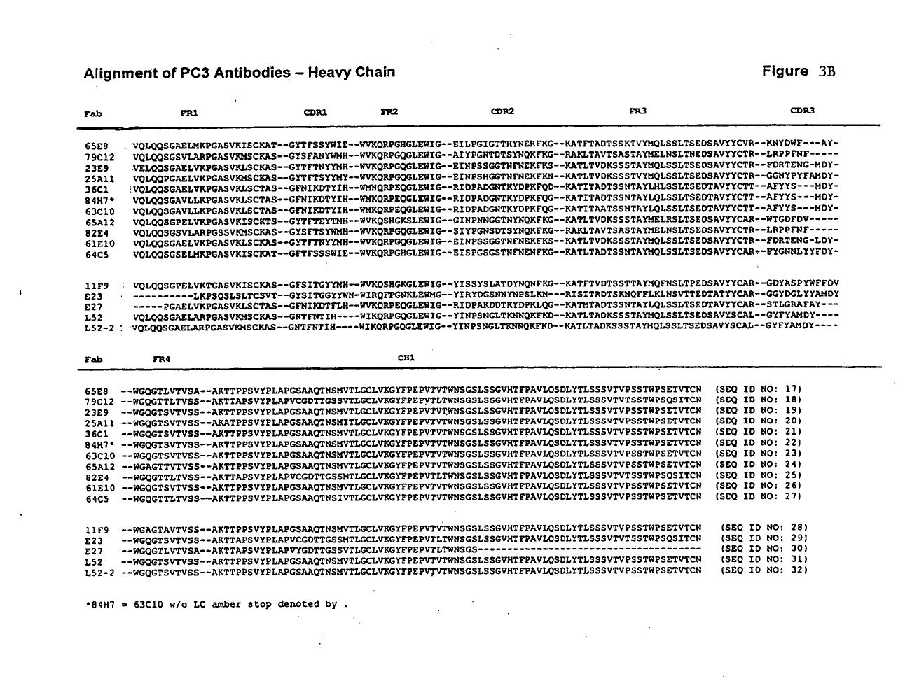

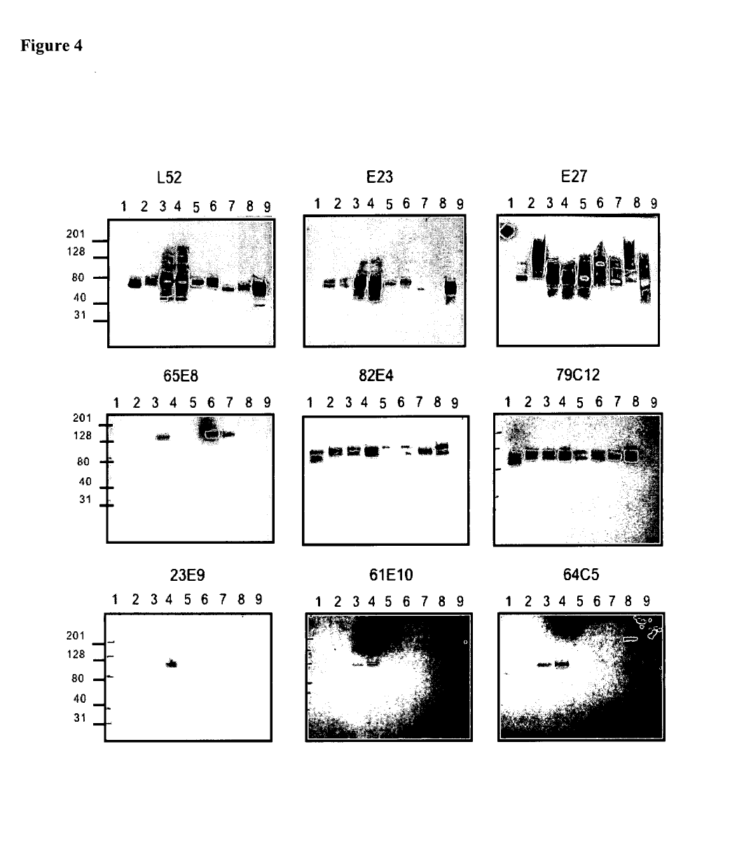

Amino acid sequence similarity of the heavy and light chain complementarity determining regions (CDRs) was used to group antibodies (FIGS. 3A-B, Table 3), in addition to antigen signature profiling through western blot analysis using 9 different cancer cell lines (FIG. 4). If the antibody binds to a linear epitope, western blot analysis provides a unique pattern for each antibody and reveals useful information about the antigens, including its absence or presence and molecular weight in a specific cancer cell line. Differing molecular weights seen in various cell lines probably reflects either alternative splicing or post-translational modifications in certain cell lines. From these studies, we found that all antibodies from the panning without RBC subtraction fell into three sequence groups (L52, E23, and E27) and, as noted, all bound to RBCs. Two groups having different CDRs (L52 and E23) were found to bind to the same target by antigen signature analysis. The antigen was identified by immunoprecipitation and subsequent mass spectrometric (IP/MS) analysis as CD55. The antigen signature of E27 suggests that it binds to a different antigen that is also shared on RBCs, but has not been identified to date.

TABLE 3
PC3 antigens identified by immunoprecipitation
and mass spectrometry

| Antibody | Antigen |
|---|---|
| 65E8 | CD26 (DPPIV) |
| 79C12 | Integrin alpha2/alpha3/beta1 |
| 23E9 | Integrin alpha3/beta1 |
| 25A11 | Cdcp1 |
| 36C1 | Integrin alpha3/beta1 |
| 84H7 (63C10) | Integrin alpha3/beta1 |
| 65A12 | Integrin alpha6/beta4 |
| 82E4 | Integrin alpha2/alpha3/alpha5/beta1 |
| 61E10 | Integrin alpha3/beta1 |
| 64C5 | Unknown |
| 11F9 | Unknown (P20) |
| E23 | CD55 |
| E27 | Unknown |
| L52 | CD55 |

In contrast, panning with RBC subtraction identified a total of ten unique antibodies that bound to PC-3 cells, but did not bind to RBCs. 146 clones were obtained that bind to PC3 cancer cells from a total of 4416 output clones after three rounds of whole cell panning (R3). Of 146 clones, 24 were found not to bind red blood cells. These 24 clones were sequenced. With the addition of two clones from R2 pan, a total of 10 clones were obtained with different Fab sequences (FIGS. 3A and 3B). The final 10 clones bind to PC3 cancer cells, but do not bind to human red blood cells. Of these 10 clones, two do not bind PrEC (Table 4).

TABLE 4
PrEC Binding

| Antibody | PrEC ELISA |
|---|---|
| 65E8 | − |
| 79C12 | +/− |
| 23E9 | + |
| 25A11 | − |
| 36C1 | + |
| 84H7 (63C10) | + |
| 65A12 | +/− |
| 82E4 | +/− |
| 61E10 | + |
| 64C5 | ND |

PrEC (+/−): 300 nM has <2-fold higher binding than secondary antibody alone.
PrEC (+): 300 nM has 2-to 3-fold higher binding than secondary antibody alone.

The antigen signatures of these antibodies demonstrate that in spite of sequence differences, antigen similarity can easily be determined by this method (FIG. 4). For example, although 82E4 and 79C12 have several amino acid differences in heavy and light chains, it is apparent by antigen signature that it is likely that these antibodies recognize the same antigen. Likewise, 23E9 and 61E10 also have several differences in heavy and light chain sequence, but have similar antigen signatures to each other. Four antibodies, 25A11, 36C1, 84H7 and 65A12, bind to PC-3 cells by flow cytometry but do not give a signal by Western blot, suggesting that these antibodies bind to non-linear, conformational epitopes on their respective antigens. IP/MS indicated that antibody 25A11 is specific for CDCP1 and antibody 65E8 is specific for CD26. For these antibodies, which recognize a single protein, specificity was confirmed by the transfection of antigen cDNA into CHO-K1 cells (which have a negative background for these antibodies) and subsequent flow cytometric analysis (data not shown). IP/MS further showed that 82E4 recognizes the integrin complex combinations α2/α3/α5/β1; 79C12 recognizes integrins α2/α3/β1; 23E9, 61E10, 36C1, and 84H7 all recognize α3β1; and 65A12 recognizes α6β4. The similarities in the 82E4 and 79C12 antigen signatures suggest that these antibodies bind to the same antigen, probably integrin β1, as it is able to pair with different combinations of alpha subunit. The antibodies 23E9 and 61E10 likely bind to the same antigen, α3β1, but 36C1 and 84H7 which pull down the same integrin complex by IP/MS, do not recognize a linear antigen, and so have different specificities. The antigen for 64C5 has not been determined to date.

In summary, we have demonstrated that whole-cell panning with human RBC subtraction increases the diversity of selected antibodies, in addition to identifying tumor antigens more efficiently. Among the cancer targets identified from whole-cell panning combined with our stringent RBC negative selection, CDCP1, CD26, and the integrin complexes α2β1, α3β1, α5β1, and α6β4, all have implications for tumorigenicity or cancer cell migration and/or invasion.

### EXAMPLE 5

### Materials and Methods for Examples 1-4

Reagents. Phycoerythrin (PE)-conjugated goat anti-mouse IgG, alkaline phosphatase (AP)-conjugated goat anti-mouse IgG, Histopaque, and AP substrate tablets were obtained from Sigma Chemical Corp. (St. Louis, Mo.). Unconjugated rabbit anti-mouse Fab, AP-conjugated goat anti-mouse F(ab′)₂, Super-Signal WestPico development reagents, chicken egg white avidin, and immobilized streptavidin were obtained from Pierce (Rockford, Ill.).

Cell lines. Cancer cell lines MCF-7, SK-OV3, PC-3, Du145, A-431 and Caki-1 were obtained from the American Type Culture Collection (Manassas, Va.). MDA-MB-435 was obtained from NCI (Frederick, Md.). KM12L4a was obtained from M.D. Anderson Cancer Center (Houston, Tex.). MDA-MB-435, MCF-7, SK-OV3, PC-3, and KM12L4a were grown in EMEM (Cambrex Bio Science, Walkersville, Md.) containing 1.75 mM L-glutamine, 10% FBS, 1×MEM vitamin solution, 1×MEM non-essential amino acid solution, and 0.9 mM sodium pyruvate. Du145 cells were grown in RPMI-1640 medium (Invitrogen, Carlsbad, Calif.) containing 2 mM L-glutamine, 10% FBS, 10 mM HEPES, 1 mM sodium pyruvate, glucose at 4.5 g/L, and sodium bicarbonate at 1.5 g/L. A-431 cells were grown in DMEM (Invitrogen) containing 1.5 mM L-glutamine, 10% FBS, glucose at 4.5 g/L, and sodium bicarbonate at 1.5 g/L. Caki-1 cells were grown in McCoy's 5a medium (Invitrogen) containing 1.5 mM L-glutamine, 10% FBS, and sodium bicarbonate at 2.2 g/L. The PrEC normal prostate epithelial cell line was obtained from Cambrex BioScience and cultured in PrEGM according to manufacturer's instructions.

Cell immunizations and antibody library construction. Three to five 4-6 week old Balb/c mice (Charles River Laboratory, Cambridge, Mass.) were each immunized four times with 3×10⁶ cancer cells. Three days after the final immunization, lymph nodes and spleen were collected for the construction of Fab antibody libraries by RT-PCR, and whole blood was collected for flow cytometry to evaluate the success of the immunization. For Fab library construction, total RNA was isolated from two PC-3 immunized mouse spleens using TRI reagent (Molecular Research Center, Cincinnati, Ohio). Complementary DNA (cDNA) was prepared and cloned into IgG1κ and IgG2aκ Fab phage expression vectors as described previously (Dakappagari et al., J. Immunol. 176:426-440 (2006); Wild et al., Nat. Biotechnol. 21:1305-1306 (2003)). The library sizes were 6.85×10⁹ for IgG1κ and 1.5×10¹⁰ for IgG2aκ.

Flow cytometry. Primary antibody, pre-bleeds, post-bleeds, subtracted sera (diluted at 1:100 or 1:200 with PBS) or phage-displayed mouse Fab supernatant was incubated with cancer cells, RBCs, or WBCs (˜0.5×10⁶ cells in 100 μL) on ice for 30 min. After washing twice with PBS, the cells were stained with PE-conjugated goat anti-mouse IgG and detected on a Becton Dickinson FACSCalibur flow cytometric analyzer. Secondary antibody alone was used as a control for background staining. The geometric mean of fluorescence intensity (geomean) was calculated using CellQuest software (Becton Dickinson).

Subtraction of antisera from cancer-cell immunized mice with human RBCs and WBCs. After obtaining Internal Review Board approval and informed consent, blood was drawn from healthy donors at Alexion Antibody Technologies, San Diego. WBCs and RBCs were isolated by Histopaque centrifugation following the manufacturer's instructions and assayed immediately. Antisera (100 μL) from immunized mice were diluted 1:10 in PBS and subtracted on RBCs (1.2×10⁹ cells) by gently rocking at 4° C. for 1 h. After centrifugation at 400 g for 10 min to remove RBCs, the subtraction was repeated five times with fresh RBCs. WBC subtraction was performed after RBC subtraction with a WBC pellet containing 4×10⁷ cells each time, and repeated for a total of three rounds.

Phage amplification: PC-3 Fab library DNA (IgG1κ and IgG2aκ, 10 μg each) was transformed into XL1-blue cells (Stratagene, La Jolla, Calif.). After 1 mM IPTG induction overnight, phage were purified by centrifugation with 0.25 volume of 20% PEG/2.5 M NaCl at 12,700 g, 4° C. for 20 min. The phage pellet was resuspended in PC-3 complete cell culture media containing 1% BSA and protease inhibitor (Complete EDTA-free protease inhibitors, Roche, Mannheim, Germany). Cell debris was removed by centrifugation at 1800 g for 5 min. Phage supernatant was filtered through a 0.2 μm GF-prefilter (Sartorius, Hannover, Germany) in a 3-mL syringe, then dialyzed into 1 liter of PBS.

Whole-cell panning. Adherent PC-3 cells (˜1.2×10⁷ total) were blocked in 4% milk/PBS at 37° C. for 1 h. A dialyzed phage preparation (10¹² cfu/mL) in complete media containing 1% BSA and protease inhibitors was added to the cells and gently rocked at 4° C. for 4 h. Cells were washed 5 times, 1 min at RT with 10 mL PBS, and phage particles were eluted and amplified in ER2738 as described (Siegel et al., J. Immunol. Methods 206:73-85 (1997). For each round of panning, output phage titers were determined. For RBC subtraction, dialyzed phage (4×10¹² pfu) were mixed with RBCs (5.7×10⁹ in 1% BSA/PBS) at a ratio of 700:1 and gently shaken at 4° C. for 1 h. RBC subtraction was repeated three times. For normal prostate cell subtraction, PrEC cells (˜4×10⁶) were blocked with 4% milk/PBS and incubated with phage at 4° C. for 2 h. Phage were transferred to PC-3 cells for subsequent positive panning. Round 1 was positive selection using PC-3 cells followed by subtraction. Phage libraries were subtracted on PrEC for 12 h twice (for the pan without RBC subtraction), or subtracted on RBCs for 1 h three times followed by subtraction on PrEC for 2 h twice (for the pan with RBC subtraction). In both cases, Round 2 and Round 3 were additional rounds of PC-3 positive selection.

Cell ELISA using PrEC. PrEC cells were plated into 96-well flat bottom plates and grown at 37° C. in 5% CO₂ incubator until confluent. Cells were fixed with 3.7% neutral buffered formalin in PBS at RT for 10 min, washed twice with PBS, and blocked with 1% BSA/PBS for 1 h at 37° C. Binding of Fabs was assayed in each well by adding 50 μL of phage supernatant in 100 μL FACS buffer (1×PBS, 5 mM EDTA, 2% FBS, 0.1% sodium azide) to cells for 2 h at 37° C. After washing cells twice with PBS, Fabs were detected with AP-conjugated goat anti-mouse IgG, incubated for 1 h at 37° C., then washed twice with PBS. Plates were developed with AP substrate tablets in PNPP buffer, and read at 405 nm in a Molecular Devices Vmax kinetic microplate reader.

Western blot. Forty micrograms of total protein from each cancer cell line was run on a nonreducing 4-15% gradient SDS-PAGE gel and transferred to 0.45 μm Optitran membrane (Schleicher and Schuell, Keene, N.H.). The membrane was blocked overnight in 4% milk/PBS, and incubated with purified Fab (30 nM) at RT for 2 h. After washing three times in PBS, horse-radish peroxidase (HRP)-conjugated goat anti-mouse IgG (H+L) (Bio-Rad, Hercules, Calif.) was added for 1 h at RT then washed three times in PBS, and developed using Super-Signal WestPico reagents.

Immunoprecipitation and mass spectral analysis. PC-3 cell membranes (10⁸ cell equivalents/mL) in Nonidet P-40 Lysis Buffer (1% Nonidet P-40, 50 mM Tris.HCl pH 7.5, 0.15 M NaCl, 10% glycerol plus Complete Protease Inhibitors) were incubated with 50 μg/mL chicken egg white avidin for 30 min on ice. Insoluble material was removed by centrifugation for 30 min at 150,000 g. The lysate was precleared with normal rabbit serum and protein G Sepharose beads (Amersham-Pharmacia, Piscataway, N.J.). Antigens were captured by adding biotinylated Fabs (10 μg/mL) to the lysate and incubating overnight at 4° C. Immobilized streptavidin was added (50 μL of packed beads per mL) for 3 h at 4° C. with gentle mixing, followed by five washes with Nonidet P-40 Lysis Buffer. After SDS-PAGE separation, antigen bands were cut from the gel. Trypsin digestion and peptide sequence analysis was performed by Bill Lane at the Harvard Microchemistry Facility (Boston, Mass.).

### EXAMPLE 6

### Expression of CDCP1 mRNA in Prostate Cell Lines, SCID Xenografts, and Prostate Patient Samples by RT-qPCR

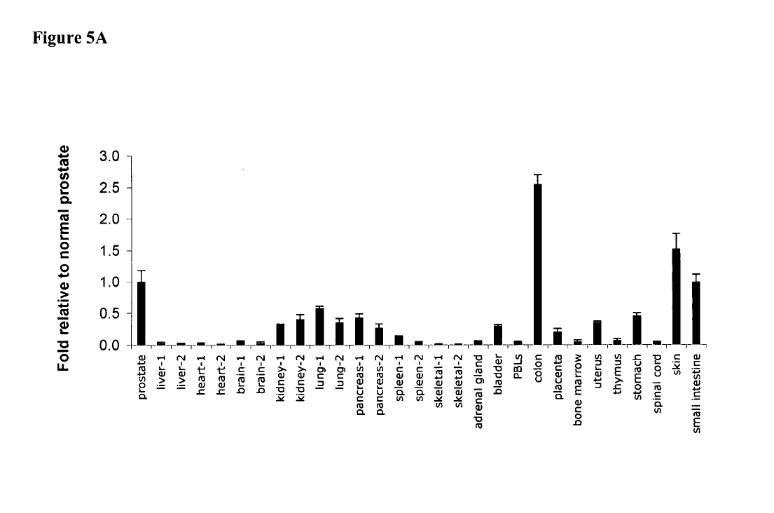

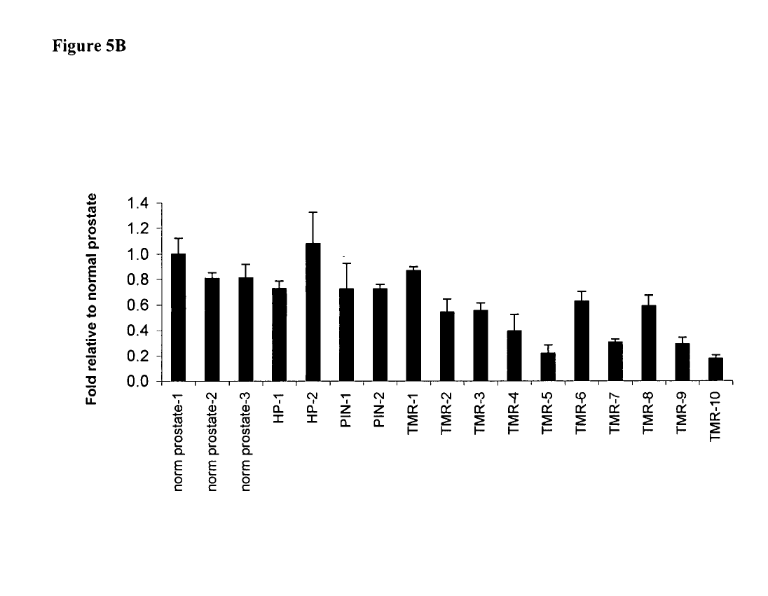

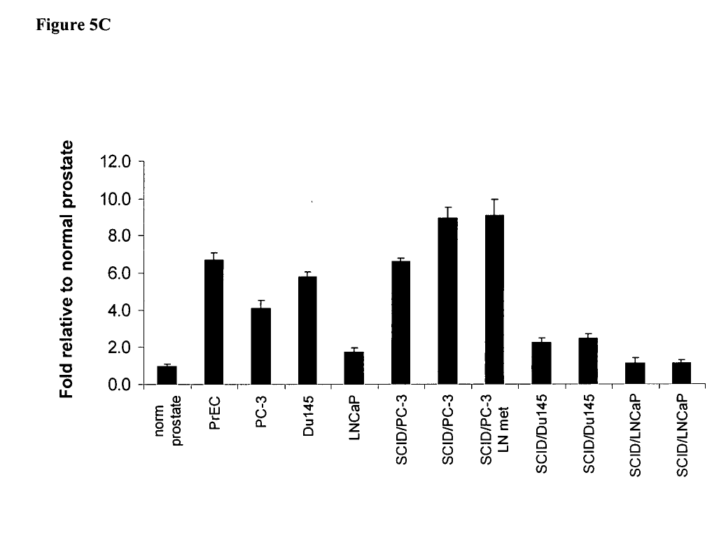

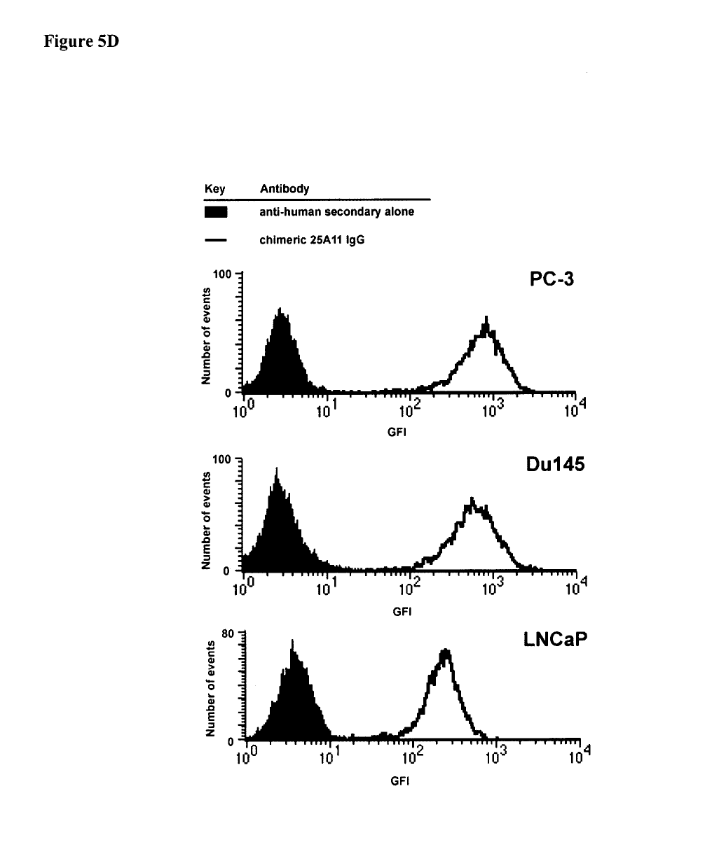

The CDCP1 mRNA expression pattern was determined by RT-qPCR for normal human tissues and prostate cancer patient samples, as well as prostate cancer cell lines and corresponding prostate cell line xenografts. All samples were internally normalized to the 18S rRNA, and the relative expression compared to normal prostate tissue was determined. In normal human tissues, the highest expression of CDCP1 was found in colon (approximately 2.5-fold higher than normal prostate), followed by skin, small intestine, and normal prostate (FIG. 5A). Lower CDCP1 expression (approximately half the level of normal prostate) was found in kidney, lung, pancreas, bladder, placenta, uterus, and stomach. In prostate cancer patient samples, the CDCP1 transcript level was approximately the same as or slightly lower than in normal prostate tissue (FIG. 5B). CDCP1 transcripts were detected in all three prostate cancer cell lines examined, PC-3, Du145, and LNCaP, as well as the corresponding SCID mouse tumor xenografts, with the PC-3 cell line and xenograft showing the highest CDCP1 expression at approximately 4- to 9-fold higher as compared to normal prostate (FIG. 5C). CDCP1 protein expression on PC-3, Du145, and LNCaP cell lines was confirmed by flow cytometry with the chimeric 25A11 monoclonal antibody (FIG. 5D).

### EXAMPLE 7

### CDCP1 Protein Expression in Prostate Cancer, Normal Human and Normal Mouse Tissues by IHC

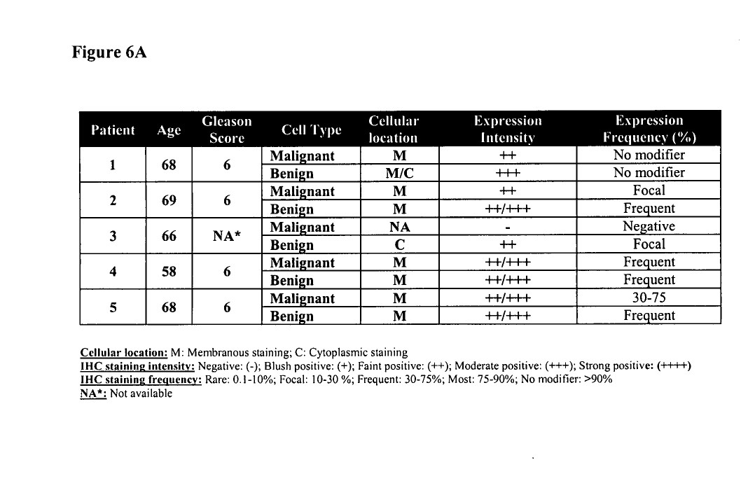

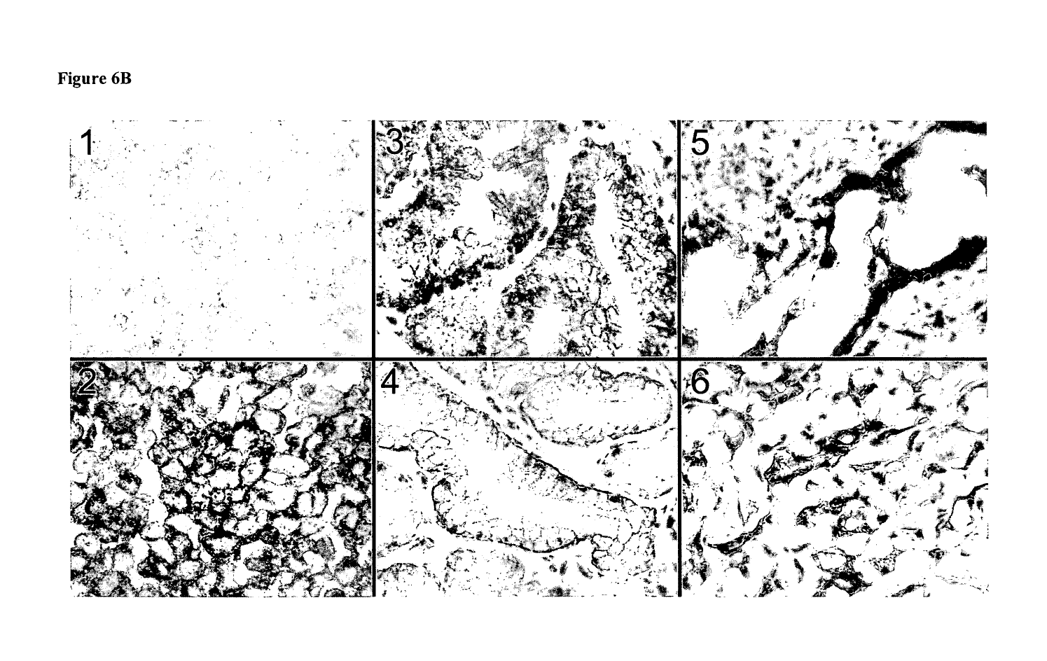

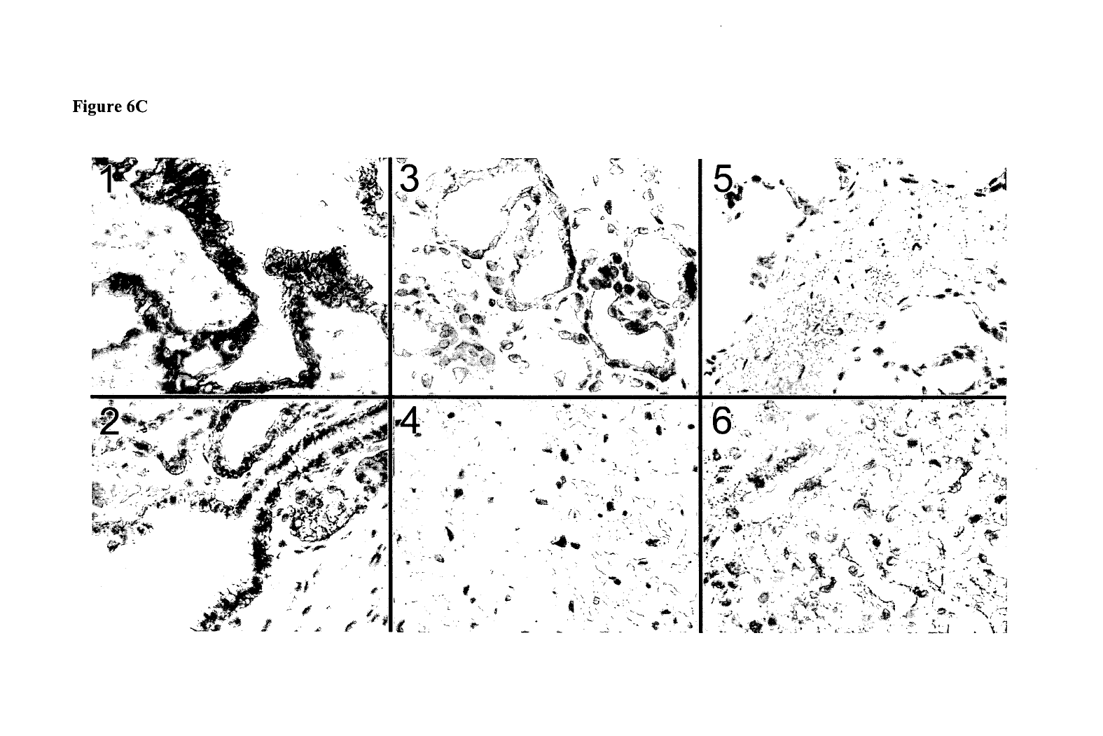

CDCP1 was found to be present on both normal prostate epithelial cells as well as on malignant cells in four of five prostate patient samples examined by IHC staining with 25A11 (data from IHC summarized in FIG. 6A). In this study, antibody 25A11 was evaluated on frozen sections of normal prostate and on samples of prostate cancer with associated benign glands. The most prominent staining was observed in benign glandular epithelium. Malignant glands were also positive, but staining was generally less prevalent and less intense than in adjacent benign glands and benign glandular epithelium in normal samples (FIG. 6B). Staining in both benign and malignant glands was predominantly membranous. Other cell types identified in this study were negative, which included inflammatory cells, endothelium, vascular smooth muscle, nerves, and prostatic fibromuscular stroma.

CDCP1 protein expression was evaluated by IHC staining with 25A11 in several additional normal human tissues, including lung, kidney, heart, spleen, liver, pancreas (FIG. 6C). Normal human colon was used as a positive control in this study (data not shown), as described in a previous publication (Hooper et al., Oncogene, 22: 1783-1794 (2003)). Moderate staining was identified in colonic epithelium, bile ducts, pancreatic ducts, and respiratory epithelium. Less intense staining was identified in a subset of renal tubular epithelium, mucous glands in the bronchi, and pancreatic islets. However, in the pancreas, many cell types were negative, including endothelium, smooth muscle, fibroblasts, and peripheral nerves. In the liver, hepatocytes showed slight staining. Heart and spleen tissues were negative. Glomeruli, including parietal epithelial cells, visceral epithelial cells, mesangial cells, and glomerular capillary endothelium, were also negative. To test for cross-reactivity of the 25A11 antibody to murine CDCP1, ch25A11 was used to evaluate IHC staining of mouse tissues. Most normal mouse tissues were negative with the exception of weak staining in mouse hepatocytes (data not shown).

### EXAMPLE 8

### 25A11 Binds to a Distinct Epitope of CDCP1 and Blocks Cell Migration and Invasion In Vitro

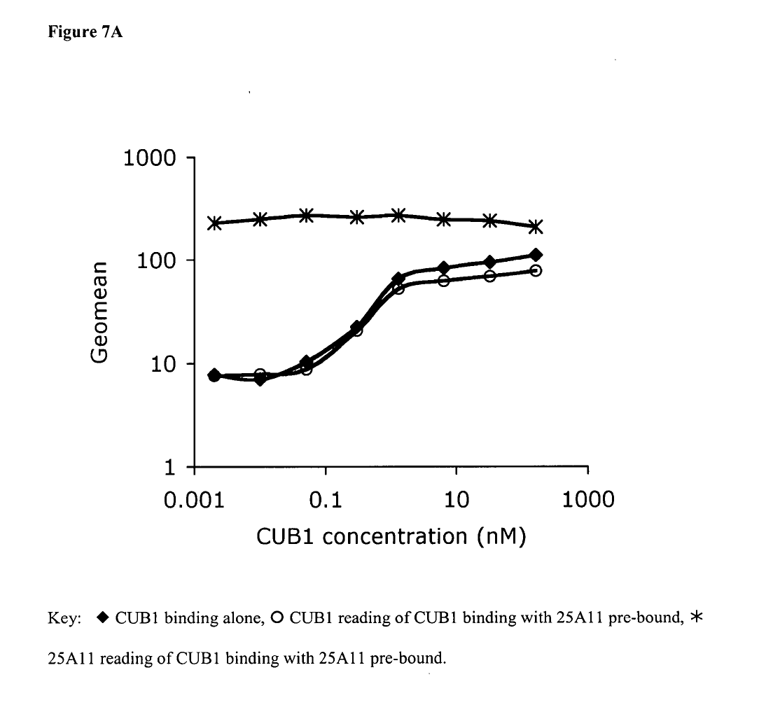

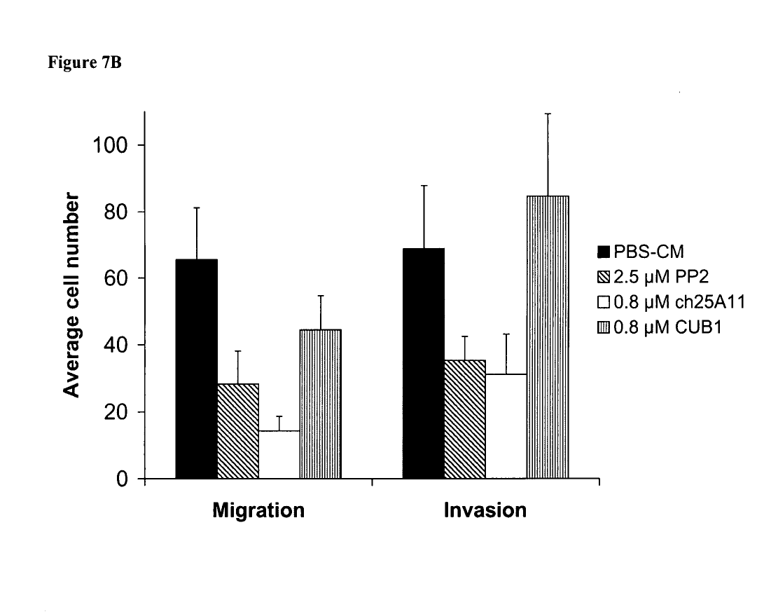

To determine if 25A11 binds to a different epitope on CDCP1 than the commercially available CUB1 antibody, originally described by Conze et al. (Conze et al., Ann N Y Acad Sci, 996:222-226 (2003)), PC-3 cells were pre-bound with 25A11 Fab at 80% saturation and the binding of CUB1 to PC-3 cells was evaluated. CUB1 binding to PC-3 cells increases with higher concentrations at the same rate, regardless of whether 25A11 is pre-bound or not (FIG. 7A), suggesting that 25A11 binds to a different epitope of CDCP1 than does CUB1.

To determine if the 25A11 antibody could inhibit cancer cell migration and invasion, assays were performed using Boyden chambers in which the effect of 25A11 on the migration/invasion of PC-3 cells through the membrane into the outer chamber was evaluated (described in the methods). Addition of the Src inhibitor PP2 was used as a positive control in this study, as it is known to block cell migration and invasion (Fan et al., J Biol Chem, 276:13240-13247 (2001)). In the cell migration assay, ch25A11-treatment at 0.8 μM resulted in a 78% inhibition of PC-3 cell migration compared to the PBS/complete media control. This amount of inhibition was superior to the 2.5 μM PP2 treatment, which gave only about 57% inhibition as compared to the PBS/complete media control. Ch25A11 at a concentration of 0.8 μM also effectively inhibited PC-3 cell invasion at levels comparable to 2.5 μM PP2, resulting in approximately 45% inhibition of invasion compared to the PBS/complete media control (FIG. 7B). The effect of CUB1 on cell migration and invasion was also evaluated; however, CUB1 had only a modest effect of 32% inhibition of cell migration, and was not found to inhibit cell invasion.

### EXAMPLE 9

### Murine and Chimeric 25A11 Antibodies Internalize on Binding and can Kill PC-3 Cells In Vitro

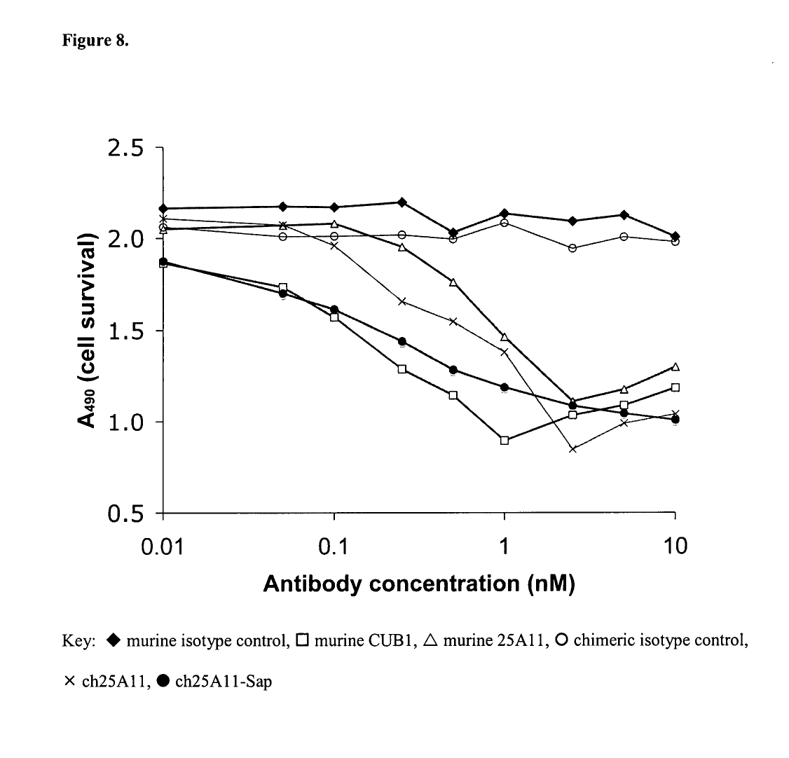

To evaluate internalization-mediated cell killing with antibody-toxin conjugates, we treated PC-3 cells with anti-CDCP1 antibodies that were either directly conjugated to saporin, or indirectly conjugated via a secondary antibody. Murine and chimeric 25A11 as well as murine CUB1 are internalized similarly as evidenced by dose-dependent PC-3 cell killing with the appropriate saporin secondary conjugates (FIG. 8), but not with the isotype control primary antibodies or the secondary saporin conjugate Goat IgG-SAP, which do not bind and internalize (data not shown). The ch25A11-Sap direct conjugate shows dose-dependent PC-3 cell killing similar to the 25A11 and CUB1 antibodies with their appropriate saporin-conjugated secondary antibodies.

### EXAMPLE 10

### Chimeric 25A11-Saporin Conjugate Directly Kills PC-3 Cells In Vivo

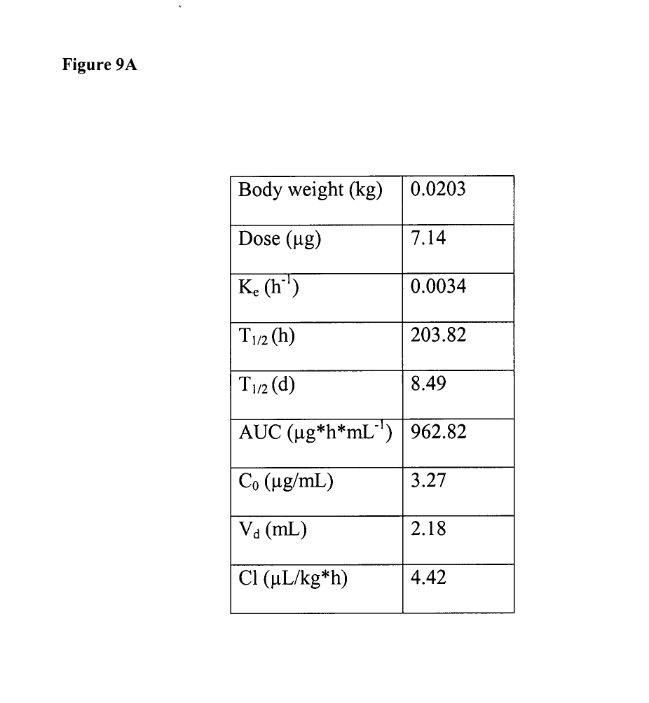

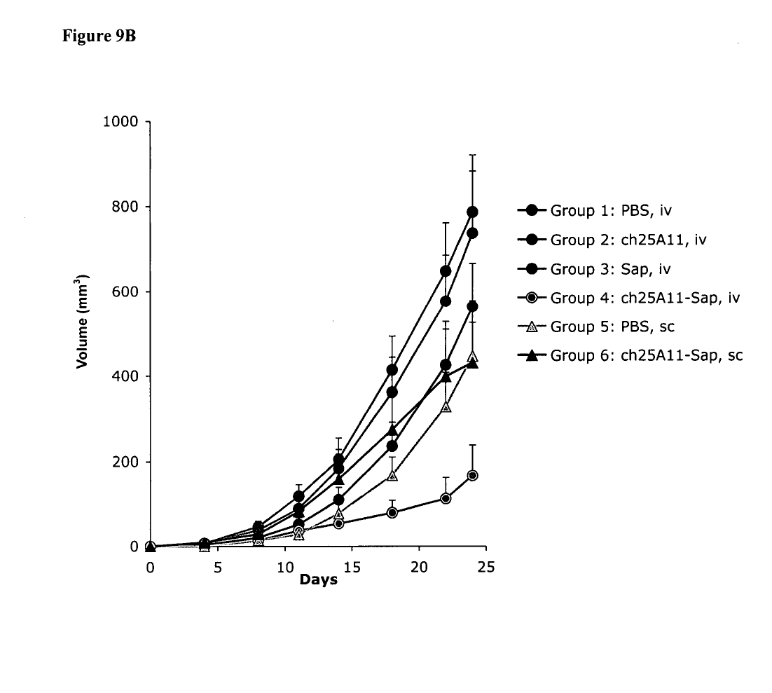

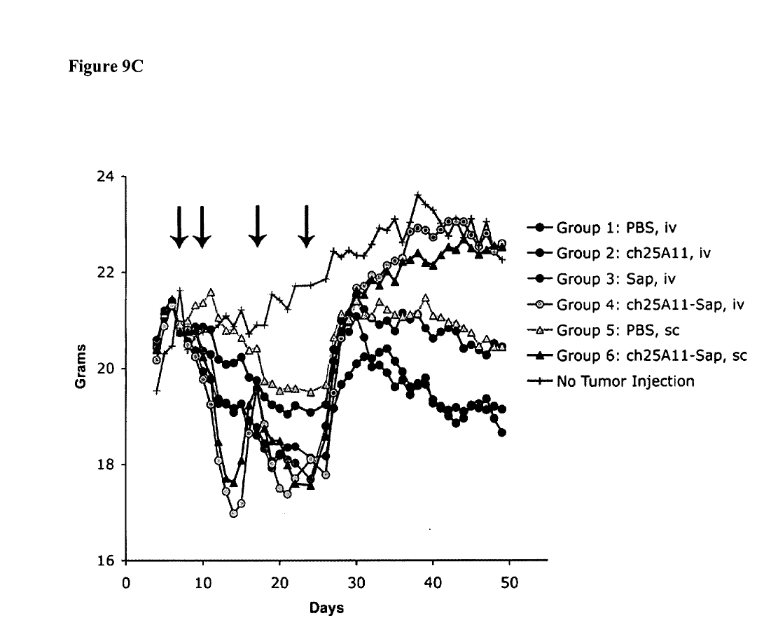

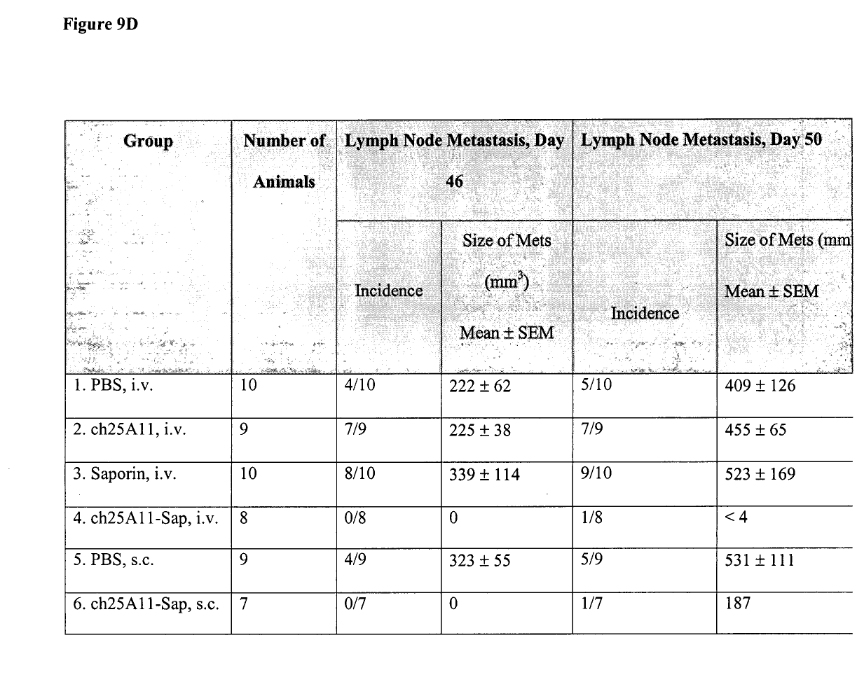

In order to evaluate in vivo cell killing with the ch25A11-Sap direct conjugate, we first performed a dosing study that demonstrated that ch25A11, or saporin treatments did not have toxicity in SCID.CB17 mice. In contrast, one to four doses of ch25A11-Sap treatment caused immediate body weight loss up to 24%, indicative of acute toxicity; however, none of the mice died (data not shown). The dosing study indicated that the optimal regimen for ch25A11-Sap was a three-dose treatment on Days 7, 10 and 17. Pharmacokinetic parameters indicate that ch25A11 has a long elimination half-life in mice of 8.4 days (FIG. 9A). The volume of distribution (V_{D}) of ch25A11 is close to the circulation volume of SCID mice at the same age, indicating adequate drug exposure. The small clearance (Cl) value, reasonable concentration at time 0 (C₀) and area under the curve (AUC) support the regimen used in the efficacy study.

In the efficacy study, the effects of ch25A11, saporin, and ch25A11-Sap were evaluated for PC-3 tumor growth inhibition and compared with PBS. The ch25A11 and saporin doses were designed to approximate the amounts of each present in the 0.4 mg/kg immunoconjugate dose. Administration of ch25A11 i.v. and saporin i.v. did not affect PC-3 tumor growth (FIG. 9B). Chimeric 25A11-Sap i.v. inhibited tumor growth approximately 66% at Day 18, 67% at Day 22, and 63% at Day 23, which were significant (p-value<0.05) by Mann-Whitney test. Chimeric 25A11-Sap s.c. did not inhibit tumor growth, suggesting possible poor bioavailability and low drug exposure at the primary tumor site by the s.c. route. The ch25A11-alone and saporin-alone groups showed slightly larger tumor burdens than the PBS control, but this was not statistically significant.

Both i.v. and s.c. administration of ch25A11-Sap caused acute toxicity, demonstrated by the considerable body weight loss one day post-injection (FIG. 9C). After primary tumor removal on Day 23, mice in both groups treated with ch25A11-Sap regained body weight similar to that of control mice without tumors by Day 35. In ch25A11-treated and saporin-treated groups, the body weight loss was caused mainly by large tumor burden and severe lymph node metastasis, but was not due to drug-related toxicity.

Lymph node metastasis of all groups was analyzed by the incidence and size of metastatic lesions (summarized in FIG. 9D). Importantly, both i.v. and s.c. ch25A11-Sap inhibited tumor metastasis, suggesting that s.c. ch25A11-Sap may not have sufficient bioavailability to inhibit primary tumor growth, but that the inhibition of metastases may be a direct result of tumor cell killing in the circulation. In summary, these data demonstrate the ability of an anti-CDCP1 immunotoxin conjugate to inhibit primary tumor growth and metastasis in vivo, and may provide therapeutic options for inhibition of metastasis in cancer patients with CDCP1-expressing tumors.

### EXAMPLE 11

### Materials and Methods for Examples 6-10

Cell lines. Prostate adenocarcinoma PC-3, prostate carcinoma lymph node metastasis LNCaP, and prostate carcinoma brain metastasis Du145 cell lines were obtained from the American Type Culture Collection (Manassas, Va.). PC-3 cells were grown in EMEM (Cambrex Bio Science, Walkersville, Md.) containing 1.75 mM L-glutamine, 10% FBS, 1×MEM Vitamin solution, IX MEM non-essential amino acid solution, and 0.9 mM sodium pyruvate. Du145 and LNCaP cells were grown in RPMI-1640 medium (Invitrogen, Carlsbad, Calif.) containing 2 mM L-glutamine, 10% FBS, 10 mM HEPES. 1 mM sodium pyruvate, glucose at 4.5 g/L, and sodium bicarbonate at 1.5 g/L. The PrEC normal prostate epithelial cell line was obtained from Cambrex Bio Science and cultured in PrEGM according to manufacturer's instructions.

Reagents and antibodies. Phage-display antibody production, antibody selection by cell-surface panning with stringent negative selection, and antigen identification for 25A11 was described in PCT/US2005/024260. Murine IgG 25A11 was made by cloning the 25A11 murine Fab into a murine IgG vector; chimeric antibodies were made by inserting variable regions of the murine Fab by overlap PCR into Fab vectors containing human constant regions, then subcloning into a human IgG vector. Antibodies used in flow cytometry, immunohistochemistry, and internalization assays included chimeric 25A11 Mab (ch25A11), and murine anti-CDCP1 CUB1 (MBL, Nagoya, Japan). Isotype control antibodies used in internalization assays included an in-house chimeric antibody and murine anti-OX7 (Advanced Targeting Systems, San Diego, Calif.). Saporin-conjugated goat anti-mouse IgG (Mab-ZAP), goat anti-human IgG (Hum-ZAP), and goat IgG isotype control (Goat IgG-SAP) secondary antibodies, as well as the chimeric 25A11-saporin custom direct conjugate (ch25A11-Sap) were purchased from Advanced Targeting Systems. The ratio of toxin to antibody in the ch25A11-Sap conjugate was approximately 2:1. CUB1-zenon labeling was performed using the Zenon Mouse IgG labeling kit (Invitrogen). Src inhibitor PP2 (4-Amino-5-(4-chlorophenyl)-7-(t-butyl)pyrazolo[3,4-d]pyrimidine) was purchased from CalBiochem (San Diego, Calif.).

RNA samples. Total RNA for the normal tissue panel was purchased from BD Biosciences (Palo Alto, Calif.) and BioChain Institute Inc. (Hayward, Calif.). Total RNA from frozen sections of prostate cell lines and SCID xenografts was extracted using RNeasy mini kits (Qiagen, Chatsworth, Calif.) or TRIzol reagent (Invitrogen) according to the manufacturer's instructions. RNA from 13 prostate patient and 2 normal samples was purchased from Ardais Corporation (Lexington, Mass.). RNA from one additional prostate cancer patient sample was obtained from Asterand (Detroit, Mich.). All RNA samples were treated with DNase I, and cDNA was prepared using the High-Capacity cDNA archive kit (Applied Biosystems, Foster City, Calif.).

Real-time quantitative PCR. Relative gene expression levels were determined by RT-qPCR using 18S ribosomal RNA (rRNA) for normalization. Assays-on-Demand TaqMan probes for CDCP1 and 18S rRNA were used with TaqMan Universal PCR master mix (Applied Biosystems) for cDNA amplification. Amplification and analysis were performed using the ABI 7500 sequence detection system (Applied Biosystems).

Flow cytometry. PC-3, Du145, and LNCaP cells were incubated with 200 nM ch25A11 and stained with R-phycoerythrin (R-PE)-conjugated goat anti-human IgG (H+L) (Jackson Immunoresearch Inc., West Grove, Pa.). For unique epitope determination, PC-3 cells at 1.25×10⁶/mL in 100 μL were bound to 160 nM of murine 25A11 Fab (about 80% saturation) and stained with (R-PE)-conjugated goat anti-mouse IgG (Sigma, St. Louis, Mo.). After washing with PBS twice, Zenon-labeled CUB1 antibody was added to each reaction at 0.01, 0.3, 1.3, 6.4 32 or 160 nM and incubated on ice for 30 min. Control reactions of CUB1 binding to PC-3 cells without 25A11 pre-binding were set up accordingly. All staining was detected on a Becton Dickinson FACSCalibur flow cytometric analyzer.

Immunohistochemistry. Antibody titration experiments were conducted with murine antibody 25A11 IgG1 (for human tissues) or chimeric ch25A11 IgG1 (for mouse tissues) to establish concentrations that would result in minimal background and maximum detection of signal. Serial dilutions were performed starting at 5 ug/mL and 2.5 ug/mL on fresh-frozen tissues, respectively. The concentration of 2.5 ug/ml was chosen for the study. Antibody 25A11 was used as the primary antibody and the principal detection system consisted of DAKO Envision peroxidase labeled polymer (DAKO, Carpinteria, Calif.) with DAB as the chromagen, which produces a brown-colored deposit. Tissues were also stained with positive control antibodies (CD31 and vimentin) to ensure that the tissue antigens were preserved and accessible for IHC. Only tissues that were positive for CD31 and vimentin staining were selected for the remainder of the study. The negative control consisted of treating adjacent sections similarly but in the absence of primary antibody.

Cell migration and invasion assays. Cell migration and invasion assays were purchased from Chemicon/Millipore International (Temecula, Calif.) and performed according to the manufacturer's instructions. For the cell migration assay, an 8-μm pore size polycarbonate membrane was used to evaluate the migration of PC-3 cells through the membrane into the complete media in the outer chamber. Similarly, invasion of PC-3 cells was evaluated on 8-μm polycarbonate membrane inserts coated with a uniform layer of basement membrane matrix solution, which serves as a barrier to discriminate invasive cells from non-invasive cells. In both assays, the Src inhibitor PP2 at 2.5 μM was used as a control for inhibition of migration/invasion. Other controls included the serum-free media control for assay background and a complete-media control containing 10% FBS for the maximum cell migration or invasion readout. PC-3 cells were treated with four-fold dilutions of CUB1 and ch25A11 in PBS and tested for inhibition of cell migration and invasion. The plates were incubated for 24 h at 37° C., and cells that migrated to the lower surface were stained with crystal violet and counted (5 fields per well) on an Olympus IX70 microscope.

In vitro cytotoxicity assay. PC-3 cells were plated in PC-3 medium 96-well microplate wells at 2500 cells/90 μL. The plates were incubated for 16 h at 37° C. in the presence of 5% CO₂. Primary antibodies with either Mab-ZAP or Hum-ZAP secondary antibodies, or ch25A11-Sap with no secondary, were added in a volume of 10 μL for a final well volume of 100 μL. Goat IgG-SAP was used as a non-targeted saporin control for the Mab-ZAP and Hum-ZAP secondary antibodies (data not shown). To avoid binding and internalization of primary antibody prior to its interaction with toxin-conjugated secondary antibody, cells were incubated first with the saporin toxin-conjugated anti-mouse or anti-human IgG secondary antibody (Mab-ZAP or Hum-ZAP). Recognition and internalization of the primary antibody results in delivery of the saporin-antibody complex to the cell interior, followed by cell killing. Plates were incubated 72 h at 37° C. in the presence of 5% CO₂. Cell viability was assayed using CellTiter 96 AQueous One Solution Cell Proliferation Assay according to manufacturer's instructions (Promega, Madison, Wis.), and the plates were read at 490 nm in a Molecular Devices Vₘₐₓ microplate reader. The positive control for internalization was mAb-225 tested on both A431 (Sunada et al., Proc Natl Acad Sci USA, 83:3825-3829, (1986)) and PC-3 cell lines (data not shown).

In vivo immunotoxin studies. SCID CB17 mice were used for in vivo studies. For the dosing study using ch25A11-Sap, mice were randomized and divided into seven groups of 10 mice each that were i.v. injected as follows: 200 μL of PBS on Day 0, Day 2 and Day 5 (Group 1); 0.286 mg/kg of ch25A11 (the equivalent antibody dose of the Saporin conjugate) on Day 0 (Group 2) or Day 0, Day 2, and Day 5 (Group 3); 0.4 mg/kg of ch25A11-Sap on Day 0 (Group 4), Days 0 and 2 (Group 5), Days 0, 2 and 5 (Group 6), or Days 0, 5, 7, 9 (Group 7). Sera were collected from two mice from each group at Day (−)₂, or post injection at 2 min, 30 min, 6 h, 24 h, 3 d, 7 d, and 14 d and 21 d for a pharmacokinetic study of ch25A11 and ch25A11-Sap. The amount of ch25A11 in serum was tested by ELISA and compared with the standard curve. The pharmacokinetic (PK) parameters of ch25A11-Sap could not be obtained because of the masking effect of saporin on the antibody in the conjugate, resulting in unsuccessful capture and/or detection of the antibody in the ELISA assay. The PK parameters of ch25A11 were obtained from Group 2 by non-compartmental analysis/first order kinetics.

The spontaneously metastatic PC-3 tumor model was used to evaluate the anticancer activity of the ch25A11-saporin conjugate. Briefly, 3×10⁶ cells were subcutaneously (s.c.) injected into SCID CB17 mice on the lower back on Day 0. On Day 7, mice were randomized and divided into six groups, seven to ten mice per group. Three 200 μL i.v. or s.c. injections were given to each group on Days 7, 10 and 17 at the doses specified: Group 1, PBS alone, i.v.; Group 2, 0.286 mg/kg ch25A11 antibody alone (equivalent antibody dose of the conjugate), i.v.; Group 3, 0.014 mg/kg saporin alone (equivalent toxin dose of the conjugate), i.v.; Group 4: 0.4 mg/kg ch25A11-Sap, i.v.; Group 5, PBS, s.c. (injection into the flank region of each mouse, at least 1 cm away from the tumor site); Group 6, 0.4 mg/kg ch25A11-Sap, s.c. Primary tumors were measured twice a week to assess the anti-tumor activity of ch25A11 and ch25A11-Sap. On Day 23, primary tumors were removed from all mice. To evaluate the anti-cancer activity of ch25A11 without toxin, a post surgery treatment with ch25A11 twice a week for three weeks was added to Group 2. The same post surgery PBS treatment was added to Group 1 as a control. At Day 46 and 50, lymph node metastasis was analyzed for all groups. The body weights of mice were measured daily to assess liver toxicity in both studies.

It will be understood that various modifications may be made to the embodiments disclosed herein. For example, as those skilled in the art will appreciate, the specific sequences described herein can be altered slightly without necessarily adversely affecting the functionality of the antibody or antibody fragment. For instance, substitutions of single or multiple amino acids in the antibody sequence can frequently be made without destroying the functionality of the antibody or fragment. Thus, it should be understood that antibodies having a degree of identity greater than 70% to the specific antibodies described herein are within the scope of this disclosure. In particularly useful embodiments, antibodies having an identity greater than about 80% to the specific antibodies described herein are contemplated. In other useful embodiments, antibodies having an identity greater than about 90% to the specific antibodies described herein are contemplated. Therefore, the above description should not be construed as limiting, but merely as exemplifications of preferred embodiments. Those skilled in the art will envision other modifications within the scope and spirit of the present disclosure.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject inventions are explicitly disclosed herein, the above specification is illustrative and not restrictive. Many variations of the inventions will become apparent to those skilled in the art upon review of this specification and the claims below. The full scope of the inventions should be determined by reference to the claims, along with their full scope of equivalents, and the specification, along with such variations.

SEQUENCES

SEQ ID NO: 1) (Fab 65E8 light chain)
DIPMTQTTSSLSASLGDRVTISCRASQDISNYLNWYQQKPDGTVKLLIYY
TSRLHSGVPSRFSGSGSGTDYSLTISNLEQEDIATYFCQQGNTLPYTFGG
GTKLEIKRADAAPTVSIIFPPSSEQLTSG
SEQ ID NO: 2) (Fab 79C12 light chain)
EIVLTQSPAIMSASPGEKVTMTCSASSSVSYMYWYQQKPGSSPRLLIYDT
SNLASGVPVRFSGSGSGTSYSLTISRMEAEDAATYYCQQWSSYPLTFGAG
TKLELKRADAAPTVSIFPPSSEQLTSG
SEQ ID NO: 3) (Fab 23E9 light chain)
DNVLTQSPASLAVSPGQRATISCKASQSVDYDGDNYMNWYQQKPGQPPKL
LIYAASNLESGIPARFSGSGSGTDFTLNIHPVEEEDAATYFCQQSDEDPY
TFGGGTKLEIKRADAAPTVSIFPPSSEQLTSG
SEQ ID NO: 4) (Fab 25A11 light chain)
DIQMTQTTSSLSASLGDRVTISCRASQDISNYLNWYQQKPDGTVKLLIYY
TSRLHSGVPSRFSGSGSGTDYSLTISNLEQEDIATYFCQQGNTLPWTFGG
GTKLEIKRADAAPTVSIFPPSSEQLTSG
SEQ ID NO: 5) (Fab 36C1 light chain)
DIVMTQSQKFMSTSVGDRVSVTCKASQNVGTNVAWYQQTPGQSPKALIYS
ASYRYSGVPDRFTGSGSGTDFTLTINNVQSEDLAEYFCQQYNSYPRTFGG
GTTLEIKRADAAPTVSIFPPSSEQLTSG
SEQ ID NO: 6) (Fab 84H7 light chain)
DIVLTQSQKFMSTSVGDRVSVTCKASQNVGTNVAWYQQKPGQSPKALIYS
ASYRYSGVPDRFTGSGSGTDFTLTISNVQSEDLAEYFCQQYNSYPRTFGG
GTKLEIKRADAAPTVSIFPPSSEQLTSG
SEQ ID NO: 7) (Fab 63C10 light chain)
DVVMTQTQKFMSTSVGDRVSVTCKASQNVGTNVAWYQQKPGQSPKALIYS
ASYRYSGVPDRFTGSGSGTDFTLTINNVQSEDLAEYFCQQYNSYPRTFGG
GTTLEIKRADAAPTVSIFPPSSEQLTSG
SEQ ID NO: 8) (Fab 65A12 light chain)
DIVMTQSQKFMSTSVGDRVSVTCKASQNVGTNVAWYQQKPGQSPDALIYS
ASYRYSGVPDRFTGSGSGTDFTLTITNVQSEDLADYFCQQYNSYPLTFGS
GTKLDLKRADAAPTVSIFPPSSEQLTSG
SEQ ID NO: 9) (Fab 82E4 light chain)
EIVLTQSPAIMSASPGEKVTMTCRASSSVSYMYWYQQKPGSSPRLLIYDT
SNLASGVPARFSGSGSGTSYSLTISRMEAEDAATYYCQQWSGYPLTFGAG
TKLELKRADAAPTVSIFPPSSEQLTSG
SEQ ID NO: 10) (Fab 61E10 light chain)
DIVMTQSPASLAVSLGQRATISCKASQSVDYDGDNYMNWYQQKPGQPPKL
LIYAASNLESGIPARFSGSGSGTDFTLNIHPVEEEDAATYYCQQSNGDPW
TFGGGTKLEIKRADAAPTVSIFPPSSEQLTSG
SEQ ID NO: 11) (Fab 64C5 light chain)
DIVLTQSPASLTVSLGQRATISCRASESVDNYGISFMNWFQQKPGQPPKL
LIYAASNQGSGVPARFSGSGSGTDFSLNIHPMEEDDTAMYFCQQTKEVPY
TFGGGTKLEIKRADAAPTVSIFPPSSEQLTSG
SEQ ID NO: 12) (Fab 11F9 light chain)
DIVMSQSPSSLAVSVGEKVTMSCKSSQSLLYSSNQKNYLAWYQQKPGQSP
KLLIYWASTRESGVPDRFTGSGSGTDFTLTISSVKAEDLAVYYCQQYYSY
PFTFGSGTKLEIKRADAAPTVSIFPPSSEQLTSG
SEQ ID NO: 13) (Fab E23 light chain)
DIXMTQSPASLSVSVGETVTITCRASENIYSNLAWYQQKQGKSPQLLVYA
ATNLADGVPSRFSGSGSGTQYSLKINSLQSEDFGSYYCQHFWGTPWTFGG
GTKLEIKRADAAPTVSIFPPSSEQLTSG
SEQ ID NO: 14) (Fab E27 light chain)
DIVMTQSQKFMSTSVGDRVTVTCKASQNVGTNVVWYQQKPGHSPKALIYS
ASYRFGGVPDRFTGSGSGTDFTLTISNVQSEDLAEYFCQQYNIYPYTFGG
GTKLEIKRADAAPTVSIFPPSSEQLTSG
SEQ ID NO: 15) (Fab L52 light chain)
DIVLTQSPASLSVSLGQRATISCKASQSVDNDGISYMNWYQQKPGQPPKL
LIYAASNLGSGVPARFSGSGSGTDFSLNIHPVEEEDAATYFCQQYNGYPY
TFGGGTKLEIKRADAAPTVSIFPPSSEQLTSG
SEQ ID NO: 16) (Fab L52-2 light chain)
DNVLTQSPAIMSASPGEKVTMTCRASSSVGSSYLHWYQQKSGASPKLWIY
STSKLASGVPARFSGSGSGTSYSLTISSVEAEDAATYYCQQYSGYPLTFG
GGTKLEIKRADAAPTVSIFPPSSEQLTSG
SEQ ID NO: 17) (Fab 65E8 heavy chain through CH1)
VQLQQSGAELMKPGASVKISCKATGYTFSSYWIEWVKQRPGHGLEWIGEI
LPGIGTTHYNERFKGKATFTADTSSKTVYMQLSSLTSEDSAVYYCVRKNY
DWFAYWGQGTLVTVSAAKTTPPSVYPLAPGSAAQTNSMVTLGCLVKGYFP
EPVTVTWNSGSLSSGVHTFPAVLQSDLYTLSSSVTVPSSTWPSETVTCN
SEQ ID NO: 18) (Fab 79C12 heavy chain through CH1)
VQLQQSGSVLARPGASVKMSCKASGYSFANYWMHWVKQRPGQGLEWIGAI
YPGNTDTSYNQKFKGRAKLTAVTSATAYMELNSLTNEDSAVYYCTRLRPP
FNFWGQGTTLTVSSAKTTAPSVYPLAPVCGDTTGSSVTLGCLVKGYFPEP
VTLTWNSGSLSSGVHTFPAVLQSDLYTLSSSVTVTSSTWPSQSITCN
SEQ ID NO: 19) (Fab 23E9 heavy chain through CH1)
VELQQSGAELVKPGASVKLSCKASGYTFTNYYMHWVKQRPGQGLEWIGEI
NPSSGGTNFNEKFKSKATLTVDKSSSTAYMQLSSLTSEDSAVYYCTRFDR
TENGMDYWGQGTSVTVSSAKTTPPSVYPLAPGSAAQTNSMVTLGCLVKGY
FPEPVTVTWNSGSLSSGVHTFPAVLQSDLYTLSSSVTVPSSTWPSETVTC
N
SEQ ID NO: 20) (Fab 25A11 heavy chain through CH1)
VQLQQPGAELVKPGASVKMSCKASGYTFTSYYMYWVKQRPGQGLEWIGEI
NPSHGGTNFNEKFKNKATLTVDKSSSTVYMQLSSLTSEDSAVYYCTRGGN
YPYFAMDYWGQGTSVTVSSAKTPPSVYPLAPGSAAQTNSMITLGCLVKGY
FPEPVTVTWNSGSLSSGVHTFPAVLQSDLYTLSSSVTVPSSTWPSETVTC
N
SEQ ID NO: 21) (Fab 36C1 heavy chain through CH1)
VQLQQSGAELVKPGASVKLSCTASGFNIKDTYIHWMNQRPEQGLEWIGRI
DPADGNTKYDPKFQDKATITADTSSNTAYLHLSSLTSEDTAVYYCTTAFY
YSMDYWGQGTSVTVSSAKTTPPSVYPLAPGSAAQTNSMVTLGCLVKGYFP
EPVTVTWNSGSLSSGVHTFPAVLQSDLYTLSSSVTVPSSTWPSETVTCN
SEQ ID NO: 22) (Fab 84H7 heavy chain through CH1)
VQLQQSGAVLLKPGASVKLSCTASGFNIKDTYIHWMKQRPEQGLEWIGRI
DPADGNTKYDPKFQGKATITADTSSNTAYLQLSSLTSEDTAVYYCTTAFY
YSMDYWGQGTSVTVSSAKTTPPSVYPLAPGSAAQTNSMVTLGCLVKGYFP
EPVTVTWNSGSLSSGVHTFPAVLQSDLYTLSSSVTVPSSTWPSETVTCN
SEQ ID NO: 23) (Fab 63C10 heavy chain through CH1)
VQLQQSGAVLLKPGASVKLSCTASGFNIKDTYIHWMKQRPEQGLEWIGRI
DPADGNTKYDPKFQGKATITAATSSNTAYLQLSSLTSEDTAVYYCTTAFY
YSMDYWGQGTSVTVSSAKTTPPSVYPLAPGSAAQTNSMVTLGCLVKGYFP
EPVTVTWNSGSLSSGVHTFPAVLQSDLYTLSSSVTVPSSTWPSETVTCN
SEQ ID NO: 24) (Fab 65A12 heavy chain through CH1)
VQLQQSGPELVKPGASVKISCKTSGYTFTEYTMHWVKQSHGKSLEWIGGI
NPNNGGTNYNQKFKGKATLTVDKSSSTAYMELRSLTSEDSAVYYCARWTG
DFDVWGAGTTVTVSSAKTTPPSVYPLAPGSAAQTNSMVTLGCLVKGYFPE
PVTVTWNSGSLSSGVHTFPAVLQSDLYTLSSSVTVPSSTWPSETVTCN
SEQ ID NO: 25) (Fab 82E4 heavy chain through CH1)
VQLQQSGSVLARPGSSVKMSCKASGYSFTSYWMHWVKQRPGQGLEWIGSI
YPGNSDTSYNQKFKGRAKLTAVTSASTAYMELNSLTSEDSAVYYCTRLRP
PFNFWGQGTTLTVSSAKTTAPSVYPLAPVCGDTTGSSMTLGCLVKGYFPE
PVTLTWNSGSLSSGVHTFPAVLQSDLYTLSSSVTVTSSTWPSQSITCN
SEQ ID NO: 26) (Fab 61E10 heavy chain through CH1)
VQLQQSGAELVKPGASVKLSCKASGYTFTNYYMHWVKQRPGQGLEWIGEI
NPSSGGTNFNEKFKSKATLTVDKSSSTAYMQLSSLTSEDSAVYYCTRFDR
TENGLDYWGQGTSVTVSSAKTTPPSVYPLAPGSAAQTNSMVTLGCLVKGY
FPEPVTVTWNSGSLSSGVHTFPAVLQSDLYTLSSSVTVPSSTWPSETVTC
N
SEQ ID NO: 27) (Fab 64C5 heavy chain through CH1)
VQLQQSGSELMKPGASVKISCKATGFTFSSSWIEWVKQRPGHGLEWIGEI
SPGSGSTNFNENFKGKATLTADTSSNTAYMQLSSLTSEDSAVYYCARFYG
NNLYYFDYWGQGTTLTVSSAKTTPPSVYPLAPGSAAQTNSIVTLGCLVKG
YFPEPVTVTWNSGSLSSGVHTFPAVLQSDLYTLSSSVTVPSSTWPSETVT
CN
SEQ ID NO: 28) (Fab 11F9 heavy chain through CH1)
VQLQQSGPELVKTGASVKISCKASGFSITGYYMHWVKQSHGKGLEWIGYI
SSYSLATDYNQNFKGKATFTVDTSSTTAYMQFNSLTPEDSAVYYCARGDY
ASPYWFFDVWGAGTAVTVSSAKTTPPSVYPLAPGSAAQTNSMVTLGCLVK
GYFPEPVTVTWNSGSLSSGVHTFPAVLQSDLYTLSSSVTVPSSTWPSETV
TCN
SEQ ID NO: 29) (Fab E23 heavy chain through CH1)
LKPSQSLSLTCSVTGYSITGGYYWNWIRQFPGNKLEWMGYIRYDGSNNYN
PSLKNRISITRDTSKNQFFLKLNSVTTEDTATYYCARGGYDGLYYAMDYW
GQGTSVTVSSAKTTAPSVYPLAPVCGDTTGSSMTLGCLVKGYFPEPVTLT
WNSGSLSSGVHTFPAVLQSDLYTLSSSVTVTSSTWPSQSITCN
SEQ ID NO: 30) (Fab E27 heavy chain through CH1)
PGAELVKPGASVKLSCTASGFNIKDTFLHWVKQRPEQGLEWIGRIDPAKD
DTKYDPKLQGKATMTADTSSNTAYLQLSSLTSEDTAVYYCARSTLGRAFA
YWGQGTLVTVSAAKTTAPSVYPLAPVYGDTTGSSVTLGCLVKGYFPEPVT
LTWNSGS
SEQ ID NO: 31) (Fab L52 heavy chain through CH1)
VQLQQSGAELARPGASVKMSCKASGNTFNTIHWIKQRPGQGLEWIGYINP
SNGLTKNNQKFKDKATLTADKSSSTAYMQLSSLTSEDSAVYSCALGYFYA
MDYWGQGTSVTVSSAKTTPPSVYPLAPGSAAQTNSMVTLGCLVKGYFPEP
VTVTWNSGSLSSGVHTFPAVLQSDLYTLSSSVTVPSSTWPSETVTCN
SEQ ID NO: 32) (Fab L52-2 heavy chain through CH1)
VQLQQSGAELARPGASVKMSCKASGNTFNTIHWIKQRPGQGLEWIGYINP
SNGLTKNNQKFKDKATLTADKSSSTAYMQLSSLTSEDSAVYSCALGYFYA
MDYWGQGTSVTVSSAKTTPPSVYPLAPGSAAQTNSMVTLGCLVKGYFPEP
VTVTWNSGSLSSGVHTFPAVLQSDLYTLSSSVTVPSSTWPSETVTCN
(SEQ ID NO: 33) (CDR1 of Fab 65E8 and Fab 25A11
light chain)
RASQDISNYLN
(SEQ ID NO: 34) (CDR1 of Fab 79C12 light chain)
SASSSVSYMY
(SEQ ID NO: 35) (CDR1 of Fab 23E9 and 61E10 light
chain)
KASQSVDYDGDNYMN
(SEQ ID NO: 36) (CDR1 of 36C1 and 84H7 and 63C10
and 65A12 light chain)
KASQNVGTNVA
(SEQ ID NO: 37) (CDR1 of Fab 82E4 light chain)
RASSSVSYMY
(SEQ ID NO: 38) (CDR1 of Fab 64C5 light chain)
RASESVDNYGISFMN
(SEQ ID NO: 39) (CDR1 of Fab 11F9 light chain)
KSSQSLLYSSNQKNYLA
(SEQ ID NO: 40) (CDR1 of Fab E23 light chain)
RASENIYSNLA
(SEQ ID NO: 41) (CDR1 of Fab E27 light chain)
KASQNVGTNVV
(SEQ ID NO: 42) (CDR1 of Fab L52 light chain)
KASQSVDNDGISYMN
(SEQ ID NO: 43) (CDR1 of Fab L52-2 light chain)
RASSSVGSSYLH
(SEQ ID NO: 44) (CDR2 of Fab 65E8 and 25A11 light
chain)
YTSRLHS
(SEQ ID NO: 45) (CDR2 of Fab 79C12 and 82E4 light
chain)
DTSNLAS
(SEQ ID NO: 46) (CDR2 of Fab 23E9 and 61E10 light
chain)
AASNLES
(SEQ ID NO: 47) (CDR2 of Fab 36C1 and 84H7 and
63C10 and 65A12 light chain)
SASYRYS
(SEQ ID NO: 48) (CDR2 of Fab 64C5 light chain)
AASNQGS
(SEQ ID NO: 49) (CDR2 of Fab 11F9 light chain)
WASTRES
(SEQ ID NO: 50) (CDR2 of Fab E23 light chain)
AATNLAD
(SEQ ID NO: 51) (CDR2 of Fab E27 light chain)
SASYRFG
(SEQ ID NO: 52) (CDR2 of Fab L52 light chain)
AASNLGS
(SEQ ID NO: 53) (CDR2 of Fab L52-2 light chain)
STSKLAS
(SEQ ID NO: 54) (CDR3 of Fab 65E8 light chain)
QQGNTLPYT
(SEQ ID NO: 55) (CDR3 of Fab 79C12 light chain)
QQWSSYPLT
(SEQ ID NO: 56) (CDR3 of Fab 23E9 light chain)
QQSDEDPYT
(SEQ ID NO: 57) (CDR3 of Fab 25A11 light chain)
QQGNTLPWT
(SEQ ID NO: 58) (CDR3 of Fab 36C1 and 84H7 and
63C10 light chain)
QQYNSYPRT
(SEQ ID NO: 59) (CDR3 of Fab 65A12 light chain)
QQYNSYPLT
(SEQ ID NO: 60) (CDR3 of Fab 82E4 light chain)
QQWSGYPLT
(SEQ ID NO: 61) (CDR3 of Fab 61E10 light chain)
QQSNGDPWT
(SEQ ID NO: 62) (CDR3 of Fab 64C5 light chain)
QQTKEVPYT
(SEQ ID NO: 63) (CDR3 of Fab 11F9 light chain)
QQYYSYPFT
(SEQ ID NO: 64) (CDR3 of Fab E23 light chain)
QHFWGTPWT
(SEQ ID NO: 65) (CDR3 of Fab E27 light chain)
QQYNIYPYT
(SEQ ID NO: 66) (CDR3 of Fab L52 light chain)
QQYNGYPYT
(SEQ ID NO: 67) (CDR3 of Fab L52-2 light chain)
QQYSGYPLT
(SEQ ID NO: 68) (CDR1 of Fab 65E8 heavy chain)
GYTFSSYWIE
(SEQ ID NO: 69) (CDR1 of Fab 79C12 heavy chain)
GYSFANYWMH
(SEQ ID NO: 70) (CDR1 of Fab 23E9 and 61E10 heavy
chain)
GYTFTNYYMH
(SEQ ID NO: 71) (CDR1 of Fab 25A11 heavy chain)
GYTFTSYYMY
(SEQ ID NO: 72) (CDR1 of Fab 36C1 and 84H7 and
63C10 heavy chain)
GFNIKDTYIH
(SEQ ID NO: 73) (CDR1 of Fab 65A12 heavy chain)
GYTFTEYTMH
(SEQ ID NO: 74) (CDR1 of Fab 82E4 heavy chain)
GYSFTSYWMH
(SEQ ID NO: 75) (CDR1 of Fab 64C5 heavy chain)
GFTFSSSWIE
(SEQ ID NO: 76) (CDR1 of Fab 11F9 heavy chain)
GFSITGYYMH
(SEQ ID NO: 77) (CDR1 of Fab E23 heavy chain)
GYSITGGYYWN
(SEQ ID NO: 78) (CDR1 of Fab E27 heavy chain)
GFNIKDTFLH
(SEQ ID NO: 79) (CDR1 of Fab L52 and L52-2 heavy
chain)
GNTFNTIH
(SEQ ID NO: 80) (CDR2 of Fab 65E8 heavy chain)
EILPGIGTTHYNERFKG
(SEQ ID NO: 81) (CDR2 of Fab 79C12 heavy chain)
AIYPGNTDTSYNQKFKG
(SEQ ID NO: 82) (CDR2 of Fab 23E9 and 61E10 heavy
chain)
EINPSSGGTNFNEKFKS
(SEQ ID NO: 83) (CDR2 of Fab 25A11 heavy chain)
EINPSHGGTNFNEKFKN
(SEQ ID NO: 84) (CDR2 of Fab 36C1 heavy chain)
RIDPADGNTKYDPKFQD
(SEQ ID NO: 85) (CDR2 of Fab 84H7 and 63C10 heavy
chain)
RIDPADGNTKYDPKFQG
(SEQ ID NO: 86) (CDR2 of Fab 65A12 heavy chain)
GINPNNGGTNYNQKFKG
(SEQ ID NO: 87) (CDR2 of Fab 82E4 heavy chain)
SIYPGNSDTSYNQKFKG
(SEQ ID NO: 88) (CDR2 of Fab 64C5 heavy chain)
EISPGSGSTNFNENFKG
(SEQ ID NO: 89) (CDR2 of Fab 11F9 heavy chain)
YISSYSLATDYNQNFKG
(SEQ ID NO: 90) (CDR2 of Fab E23 heavy chain)
YIRYDGSNNYNPSLKN
(SEQ ID NO: 91) (CDR2 of Fab E27 heavy chain)
RIDPAKDDTKYDPKLQG
(SEQ ID NO: 92) (CDR2 of Fab L52 and L52-2 heavy
chain)
YINPSNGLTKNNQKLFKD
(SEQ ID NO: 93) (CDR3 of Fab 65E8 heavy chain)
KNYDWFAY
(SEQ ID NO: 94) (CDR3 of Fab 79C12 heavy chain)
LRPPFNF
(SEQ ID NO: 95) (CDR3 of Fab 23E9 heavy chain)
FDRTENGMDY
(SEQ ID NO: 96) (CDR3 of Fab 25A11 heavy chain)
GGNYPYFAMDY
(SEQ ID NO: 97) (CDR3 of Fab 36C1 and 84H7 and
63C10 heavy chain)
AFYYSMDY
(SEQ ID NO: 98) (CDR3 of Fab 65A12 heavy chain)
WTGDFDV
(SEQ ID NO: 99) (CDR3 of Fab 61E10 heavy chain)
FDRTENGLDY
(SEQ ID NO: 100) (CDR3 of Fab 64C5 heavy chain)
FYGNNLYYFDY
(SEQ ID NO: 101) (CDR3 of Fab 11F9 heavy chain)
GDYASPYWFFDV
(SEQ ID NO: 102) (CDR3 of Fab E23 heavy chain)
GGYDGLYYAMDY
(SEQ ID NO: 103) (CDR3 of Fab E27 heavy chain)
STLGRAFAY
(SEQ ID NO: 104) (CDR3 of Fab L52 and L52-2 heavy
chain)
GYFYAMDY
SEQ ID NO: 105) (Variable region of Fab 25A11
light chain)
DIQMTQTTSSLSASLGDRVTISCRASQDISNYLNWYQQKPDGTVKLLIYY
TSRLHSGVPSRFSGSGSGTDYSLTISNLEQEDIATYFCQQGNTLPWT
SEQ ID NO: 106) (Variable region of Fab 25A11
heavy chain)
VQLQQPGAELVKPGASVKMSCKASGYTFTSYYMYWVKQRPGQGLEWIGEI
NPSHGGTNFNEKFKNKATLTVDKSSSTVYMQLSSLTSEDSAVYYCTRGGN
YPYFAMDYWGQGTSVTVSS
SEQ ID NO: 107)
DIQMTQTTSSLSASLGDRVTISCRASQDISNYLNWYQQKPDGTVKLLIYY
TSRLHSGVPSRFSGSGSGTDYSLTISNLEQEDIATYFCQQGNTLPWTFGG
GTKLEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC
SEQ ID NO: 108)
VQLQQPGAELVKPGASVKMSCKASGYTFTSYYMYWVKQRPGQGLEWIGEI
NPSHGGTNFNEKFKNKATLTVDKSSSTVYMQLSSLTSEDSAVYYCTRGGN
YPYFAMDYWGQGTSVTVSSASTKGPSVFPLAPSSKSTSGGTAALGCLVKD
YFPEPVTVSWNSGALTSGVHTFPAVLQSSGLYSLSSVVTVPSSSLGTQTY
ICNVNHKPSNTKVDKRVEPKSCDKTHTCPPCPAPELLGGPSVFLFPPKPK
DTLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQV
YTLPPSREEMTKNQVSLTCLVKGFYPSDIAVEWESNGQPENNYKTTPPVL
DSDGSFFLYSKLTVDKSRWQQGNVFSCSVMHEALHNHYTQKSLSLSPGK

1. An antibody or antigen-binding fragment thereof that binds CUB-domain-containing protein 1 (CDCP1), wherein the antibody is conjugated to a cytotoxic agent.

2. The antibody or antigen-binding fragment thereof according to claim 1 wherein the cytotoxic agent is toxic to a CDCP1-positive cell.

3. The antibody or antigen-binding fragment thereof according to claim 1 wherein said antibody or antibody fragment is selected from the group consisting of a polyclonal antibody, a monoclonal antibody or antibody fragment, a recombinant antibody, a diabody, a chimerized or chimeric antibody or antibody fragment, a humanized antibody or antibody fragment, a deimmunized human antibody or antibody fragment, a fully human antibody or antibody fragment, a single chain antibody, an Fv, an Fd, an Fab, an Fab′, and an F(ab′)₂.

4. The antibody or antigen-binding fragment thereof according to claim 1 wherein said antibody is a monoclonal antibody.

5. The antibody or antigen-binding fragment thereof according to claim 1 wherein the cytotoxic agent is selected from the group consisting of a compound that emits radiation, diphtheria A chain, nonbinding active fragments of diphtheria toxin, exotoxin A chain, ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, saponaria officinalis inhibitor, gelonin, mitogellin, restrictocin, phenomycin, enomycin and the tricothecenes, vinblastine, 4-desacetylvinblastine, vincristine, leurosidine, vindesine, and saporin.

6. The antibody or antigen-binding fragment thereof according to claim 5 wherein the cytotoxic agent is saporin.

7. The antibody or antigen-binding fragment thereof according to claim 5 wherein said antibody is conjugated to a cytotoxic agent through a linker which releases the cytotoxic agent inside CDCP1-positive cells.

8. The antibody or antigen-binding fragment thereof according to claim 1 comprising an altered constant region, wherein said antibody or antigen-binding fragment exhibits increased effector function relative to an anti-CDCP1 antibody with a native constant region.

9. The antibody or antigen-binding fragment thereof according to claim 8 wherein increased effector function comprises one or more properties of the following group:
a) increased antibody-dependent cell-mediated cytotoxicity (ADCC), and
b) increased complement dependent cytotoxicity (CDC), compared to an anti-CDCP1 antibody with a native constant region.

10. The antibody or antigen-binding fragment thereof according to claim 1 wherein said antibody has an anti-cancer activity.

11. The antibody or antigen-binding fragment thereof according to claim 10 wherein said anti-cancer activity is selected from the group consisting of inhibiting tumor growth, inhibiting cancer cell proliferation, inhibiting cancer cell migration, inhibiting metastasis of cancer cells, inhibiting angiogenesis, and causing tumor cell death.

12. The antibody or antigen-binding fragment thereof according to claim 1 wherein said antibody i) blocks the interaction between CDCP1 and an interacting protein selected from the group consisting of N-cadherin, P-cadherin, syndecan 1, syndecan 4, and MT-SP1, or ii) blocks Src signaling and cancer cell metastasis.

13. The antibody or antigen-binding fragment thereof according to claim 1 wherein said antibody or antigen-binding fragment thereof binds the extracellular domain of CDCP1.

14. The antibody or antigen-binding fragment thereof according to claim 1 wherein said antibody or antigen-binding fragment thereof comprises a heavy chain variable region and a light chain variable region,
wherein the heavy chain variable region comprises one or more CDR regions having an amino acid sequence selected from the group consisting of SEQ ID NO:71, SEQ ID NO:83, or SEQ ID NO:96, and
wherein the light chain variable region comprises one or more CDR regions having an amino acid sequence selected from the group consisting of SEQ ID NO:33, SEQ ID NO:44, or SEQ ID NO:57.

15. The antibody or antigen-binding fragment thereof according to claim 14 wherein said antibody or antigen-binding fragment thereof comprises a heavy chain variable region and a light chain variable region,
wherein the heavy chain variable region comprises SEQ ID NO:106 and the light chain variable region comprises SEQ ID NO:105.

16. The antibody or antigen-binding fragment thereof according to claim 15 wherein said antibody or antigen-binding fragment thereof comprises a heavy chain and a light chain, wherein the heavy chain comprises SEQ ID NO:108 and the light chain comprises SEQ ID NO:107.

17. The antibody or antigen-binding fragment thereof according to claim 1 further comprising a prostate cancer targeting agent.

18. The antibody or antigen-binding fragment thereof according to claim 17 wherein the targeting agent is a peptide.

19. The antibody or antigen-binding fragment thereof according to claim 17 wherein the targeting agent is an aptamer.

20. The antibody or antigen-binding fragment thereof according to claim 1 wherein the antibody competitively inhibits binding of a CDCP1 polypeptide to an antibody comprising a sequence selected from the group consisting of SEQ ID NOs:105 and 106.

21. A method of treating prostate cancer in a mammal comprising administering to said mammal a therapeutically effective amount of an antibody that binds CDCP1, wherein the antibody is conjugated to a cytotoxin.

22. The method of claim 21 wherein the antibody conjugated to a cytotoxic agent is toxic to a CDCP1-positive cell.

23. The method of claim 21 wherein said antibody or antigen-binding fragment thereof is selected from the group consisting of a polyclonal antibody, a monoclonal antibody or antibody fragment, a diabody, a chimerized or chimeric antibody or antibody fragment, a humanized antibody or antibody fragment, a deimmunized human antibody or antibody fragment, a fully human antibody or antibody fragment, a single chain antibody, an Fv, an Fd, an Fab, an Fab′, and an F(ab′)₂.

24. The method of claim 21 wherein said antibody or antigen-binding fragment thereof is a monoclonal antibody.

25. The method of claim 21 wherein the cytotoxic agent is selected from the group consisting of a compound that emits radiation, diphtheria A chain, nonbinding active fragments of diphtheria toxin, exotoxin A chain, ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, saponaria officinalis inhibitor, gelonin, mitogellin, restrictocin, phenomycin, enomycin and the tricothecenes, vinblastine, 4-desacetylvinblastine, vincristine, leurosidine, vindesine and saporin.

26. The method of claim 25 wherein the cytotoxic agent is saporin.

27. The method of claim 21 wherein said antibody is conjugated to a cytotoxic agent through a linker which releases the cytotoxic agent inside CDCP1-positive cells.

28. The method of claim 21 wherein said antibody or antigen-binding fragment thereof comprises an altered constant region, wherein said antibody or antigen-binding fragment exhibits increased effector function relative to an anti-CDCP1 antibody with a native constant region.

29. The method of claim 28 wherein increased effector function comprises one or more properties of the following group:
a) increased antibody-dependent cell-mediated cytotoxicity (ADCC), and
b) increased complement dependent cytotoxicity (CDC), compared to an anti-CDCP1 antibody with a native constant region.

30. The method of claim 21 wherein said antibody or antigen-binding fragment thereof is administered chronically to said mammal.

31. The method of claim 21 wherein said antibody or antigen-binding fragment thereof is administered systemically to said mammal.

32. The method of claim 21 wherein said antibody or antigen-binding fragment thereof is administered locally to said mammal.

33. The method of claim 21 wherein said antibody or antigen-binding fragment thereof has an anti-cancer activity.

34. The method of claim 21 wherein said anti-cancer activity is selected from the group consisting of inhibiting tumor growth, inhibiting cancer cell proliferation, inhibiting cancer cell migration, inhibiting cancer cell adhesion, inhibiting metastasis of cancer cells, inhibiting angiogenesis, and causing tumor cell death.

35. The method of claim 21 wherein said antibody or antigen-binding fragment thereof i) blocks the interaction between CDCP1 and an interacting protein selected from the group consisting of N-cadherin, P-cadherin, syndecan 1, syndecan 4, and MT-SP1, or ii) blocks Src signaling and cancer cell metastasis.

36. The method of claim 21 further comprising administering a chemotherapeutic agent to said mammal.

37. The method of claim 36 wherein said chemotherapeutic agent and said antibody that binds CDCP1 are administered serially.

38. The method of claim 36 wherein said chemotherapeutic agent and said antibody that binds CDCP1 are administered simultaneously.

39. The method of claim 21 wherein said cancer is prostate cancer.

40. The method of claim 21 wherein said mammal is a human.

41. The method of claim 21 wherein said antibody or antigen-binding fragment thereof binds the extracellular domain of CDCP1.

42. The method of claim 21 wherein said antibody or antigen-binding fragment thereof comprises a heavy chain variable region and a light chain variable region,
wherein the heavy chain variable region comprises one or more CDR regions having an amino acid sequence selected from the group consisting of SEQ ID NO:71, SEQ ID NO:83, or SEQ ID NO:96, and
wherein the light chain variable region comprises one or more CDR regions having an amino acid sequence selected from the group consisting of SEQ ID NO:33, SEQ ID NO:44, or SEQ ID NO:57.

43. The method of claim 21 wherein said antibody or antigen-binding fragment thereof comprises a heavy chain variable region and a light chain variable region, wherein the heavy chain variable region comprises SEQ ID NO:106 and the light chain variable region comprises SEQ ID NO:105.

44. The method of claim 43 wherein said antibody or antigen-binding fragment thereof comprises a heavy chain and a light chain,
wherein the heavy chain comprises SEQ ID NO:108 and the light chain comprises SEQ ID NO:107.

45. The method of claim 21 further comprising a prostate cancer targeting agent.

46. The method of claim 45 wherein the targeting agent is a peptide.

47. The method of claim 45 wherein the targeting agent is an aptamer.

48. The method of claim 21 wherein the antibody competitively inhibits binding of a CDCP1 polypeptide to an antibody comprising a sequence selected from SEQ ID NOs:105 or 106.


SEQUENCE LISTING (108 sequences)

SEQ ID NO: 1 - protein, 130 aa, Murine
SRDIPMTQTTSSLSASLGDRVTISCRASQDISNYLNWYQQKPDGTVKLLIYYTSRLHSGVPSRFSGSGSGTDYSLTISNLEQEDIATYFCQQGNTLPYTFGGGTKLEIKRADAAPTVSIFPPSSEQLTSG

SEQ ID NO: 2 - protein, 129 aa, Murine
SREIVLTQSPAIMSASPGEKVTMTCSASSSVSYMYWYQQKPGSSPRLLIYDTSNLASGVPVRFSGSGSGTSYSLTISRMEAEDAATYYCQQWSSYPLTFGAGTKLELKRADAAPTVSIFPPSSEQLTSG

SEQ ID NO: 3 - protein, 134 aa, Murine
SRDNVLTQSPASLAVSPGQRATISCKASQSVDYDGDNYMNWYQQKPGQPPKLLIYAASNLESGIPARFSGSGSGTDFTLNIHPVEEEDAATYFCQQSDEDPYTFGGGTKLEIKRADAAPTVSIFPPSSEQLTSG

SEQ ID NO: 4 - protein, 130 aa, Murine
SRDIQMTQTTSSLSASLGDRVTISCRASQDISNYLNWYQQKPDGTVKLLIYYTSRLHSGVPSRFSGSGSGTDYSLTISNLEQEDIATYFCQQGNTLPWTFGGGTKLEIKRADAAPTVSIFPPSSEQLTSG

SEQ ID NO: 5 - protein, 130 aa, Murine
SRDIVMTQSQKFMSTSVGDRVSVTCKASQNVGTNVAWYQQTPGQSPKALIYSASYRYSGVPDRFTGSGSGTDFTLTINNVQSEDLAEYFCQQYNSYPRTFGGGTTLEIKRADAAPTVSIFPPSSEQLTSG

SEQ ID NO: 6 - protein, 130 aa, Murine
SRDIVLTQSQKFMSTSVGDRVSVTCKASQNVGTNVAWYQQKPGQSPKALIYSASYRYSGVPDRFTGSGSGTDFTLTISNVQSEDLAEYFCQQYNSYPRTFGGGTKLEIKRADAAPTVSIFPPSSEQLTSG

SEQ ID NO: 7 - protein, 130 aa, Murine
SRDVVMTQTQKFMSTSVGDRVSVTCKASQNVGTNVAWYQQKPGQSPKALIYSASYRYSGVPDRFTGSGSGTDFTLTINNVQSEDLAEYFCQQYNSYPRTFGGGTTLEIKRADAAPTVSIFPPSSEQLTSG

SEQ ID NO: 8 - protein, 130 aa, Murine
SRDIVMTQSQKFMSTSVGDRVSVTCKASQNVGTNVAWYQQKPGQSPDALIYSASYRYSGVPDRFTGSGSGTDFTLTITNVQSEDLADYFCQQYNSYPLTFGSGTKLDLKRADAAPTVSIFPPSSEQLTSG

SEQ ID NO: 9 - protein, 129 aa, Murine
SREIVLTQSPAIMSASPGEKVTMTCRASSSVSYMYWYQQKPGSSPRLLIYDTSNLASGVPARFSGSGSGTSYSLTISRMEAEDAATYYCQQWSGYPLTFGAGTKLELKRADAAPTVSIFPPSSEQLTSG

SEQ ID NO: 10 - protein, 134 aa, Murine
SRDIVMTQSPASLAVSLGQRATISCKASQSVDYDGDNYMNWYQQKPGQPPKLLIYAASNLESGIPARFSGSGSGTDFTLNIHPVEEEDAATYYCQQSNGDPWTFGGGTKLEIKRADAAPTVSIFPPSSEQLTSG

SEQ ID NO: 11 - protein, 134 aa, Murine
SRDIVLTQSPASLTVSLGQRATISCRASESVDNYGISFMNWFQQKPGQPPKLLIYAASNQGSGVPARFSGSGSGTDFSLNIHPMEEDDTAMYFCQQTKEVPYTFGGGTKLEIKRADAAPTVSIFPPSSEQLTSG

SEQ ID NO: 12 - protein, 136 aa, Murine
SRDIVMSQSPSSLAVSVGEKVTMSCKSSQSLLYSSNQKNYLAWYQQKPGQSPKLLIYWASTRESGVPDRFTGSGSGTDFTLTISSVKAEDLAVYYCQQYYSYPFTFGSGTKLEIKRADAAPTVSIFPPSSEQLTSG

SEQ ID NO: 13 - protein, 130 aa, Murine
  VARIANT 2, 5: Xaa = Any Amino Acid
SXDIXMTQSPASLSVSVGETVTITCRASENIYSNLAWYQQKQGKSPQLLVYAATNLADGVPSRFSGSGSGTQYSLKINSLQSEDFGSYYCQHFWGTPWTFGGGTKLEIKRADAAPTVSIFPPSSEQLTSG

SEQ ID NO: 14 - protein, 130 aa, Murine
SRDIVMTQSQKFMSTSVGDRVTVTCKASQNVGTNVVWYQQKPGHSPKALIYSASYRFGGVPDRFTGSGSGTDFTLTISNVQSEDLAEYFCQQYNIYPYTFGGGTKLEIKRADAAPTVSIFPPSSEQLTSG

SEQ ID NO: 15 - protein, 134 aa, Murine
SRDIVLTQSPASLSVSLGQRATISCKASQSVDNDGISYMNWYQQKPGQPPKLLIYAASNLGSGVPARFSGSGSGTDFSLNIHPVEEEDAATYFCQQYNGYPYTFGGGTKLEIKRADAAPTVSIFPPSSEQLTSG

SEQ ID NO: 16 - protein, 131 aa, Murine
SRDNVLTQSPAIMSASPGEKVTMTCRASSSVGSSYLHWYQQKSGASPKLWIYSTSKLASGVPARFSGSGSGTSYSLTISSVEAEDAATYYCQQYSGYPLTFGGGTKLEIKRADAAPTVSIFPPSSEQLTSG

SEQ ID NO: 17 - protein, 201 aa, Murine
LEVQLQQSGAELMKPGASVKISCKATGYTFSSYWIEWVKQRPGHGLEWIGEILPGIGTTHYNERFKGKATFTADTSSKTVYMQLSSLTSEDSAVYYCVRKNYDWFAYWGQGTLVTVSAAKTTPPSVYPLAPGSAAQTNSMVTLGCLVKGYFPEPVTVTWNSGSLSSGVHTFPAVLQSDLYTLSSSVTVPSSTWPSETVTCN

SEQ ID NO: 18 - protein, 199 aa, Murine
LEVQLQQSGSVLARPGASVKMSCKASGYSFANYWMHWVKQRPGQGLEWIGAIYPGNTDTSYNQKFKGRAKLTAVTSATAYMELNSLTNEDSAVYYCTRLRPPFNFWGQGTTLTVSSAKTTAPSVYPLAPVCGDTTGSSVTLGCLVKGYFPEPVTLTWNSGSLSSGVHTFPAVLQSDLYTLSSSVTVTSSTWPSQSITCN

SEQ ID NO: 19 - protein, 203 aa, Murine
LEVELQQSGAELVKPGASVKLSCKASGYTFTNYYMHWVKQRPGQGLEWIGEINPSSGGTNFNEKFKSKATLTVDKSSSTAYMQLSSLTSEDSAVYYCTRFDRTENGMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCN

SEQ ID NO: 20 - protein, 203 aa, Murine
LEVQLQQPGAELVKPGASVKMSCKASGYTFTSYYMYWVKQRPGQGLEWIGEINPSHGGTNFNEKFKNKATLTVDKSSSTVYMQLSSLTSEDSAVYYCTRGGNYPYFAMDYWGQGTSVTVSSAKTPPSVYPLAPGSAAQTNSMITLGCLVKGYFPEPVTVTWNSGSLSSGVHTFPAVLQSDLYTLSSSVTVPSSTWPSETVTCN

SEQ ID NO: 21 - protein, 201 aa, Murine
LEVQLQQSGAELVKPGASVKLSCTASGFNIKDTYIHWMNQRPEQGLEWIGRIDPADGNTKYDPKFQDKATITADTSSNTAYLHLSSLTSEDTAVYYCTTAFYYSMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCN

SEQ ID NO: 22 - protein, 201 aa, Murine
LEVQLQQSGAVLLKPGASVKLSCTASGFNIKDTYIHWMKQRPEQGLEWIGRIDPADGNTKYDPKFQGKATITADTSSNTAYLQLSSLTSEDTAVYYCTTAFYYSMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCN

SEQ ID NO: 23 - protein, 201 aa, Murine
LEVQLQQSGAVLLKPGASVKLSCTASGFNIKDTYIHWMKQRPEQGLEWIGRIDPADGNTKYDPKFQGKATITAATSSNTAYLQLSSLTSEDTAVYYCTTAFYYSMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCN

SEQ ID NO: 24 - protein, 200 aa, Murine
LEVQLQQSGPELVKPGASVKISCKTSGYTFTEYTMHWVKQSHGKSLEWIGGINPNNGGTNYNQKFKGKATLTVDKSSSTAYMELRSLTSEDSAVYYCARWTGDFDVWGAGTTVTVSSAKTTPPSVYPLAPGSAAQTNSMVTLGCLVKGYFPEPVTVTWNSGSLSSGVHTFPAVLQSDLYTLSSSVTVPSSTWPSETVTCN

SEQ ID NO: 25 - protein, 200 aa, Murine
LEVQLQQSGSVLARPGSSVKMSCKASGYSFTSYWMHWVKQRPGQGLEWIGSIYPGNSDTSYNQKFKGRAKLTAVTSASTAYMELNSLTSEDSAVYYCTRLRPPFNFWGQGTTLTVSSAKTTAPSVYPLAPVCGDTTGSSMTLGCLVKGYFPEPVTLTWNSGSLSSGVHTFPAVLQSDLYTLSSSVTVTSSTWPSQSITCN

SEQ ID NO: 26 - protein, 203 aa, Murine
LEVQLQQSGAELVKPGASVKLSCKASGYTFTNYYMHWVKQRPGQGLEWIGEINPSSGGTNFNEKFKSKATLTVDKSSSTAYMQLSSLTSEDSAVYYCTRFDRTENGLDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCN

SEQ ID NO: 27 - protein, 204 aa, Murine
LEVQLQQSGSELMKPGASVKISCKATGFTFSSSWIEWVKQRPGHGLEWIGEISPGSGSTNFNENFKGKATLTADTSSNTAYMQLSSLTSEDSAVYYCARFYGNNLYYFDYWGQGTTLTVSSAKTTPPSVYPLAPGSAAQTNSIVTLGCLVKGYFPEPVTVTWNSGSLSSGVHTFPAVLQSDLYTLSSSVTVPSSTWPSETVTCN

SEQ ID NO: 28 - protein, 205 aa, Murine
LEVQLQQSGPELVKTGASVKISCKASGFSITGYYMHWVKQSHGKGLEWIGYISSYSLATDYNQNFKGKATFTVDTSSTTAYMQFNSLTPEDSAVYYCARGDYASPYWFFDVWGAGTAVTVSSAKTTPPSVYPLAPGSAAQTNSMVTLGCLVKGYFPEPVTVTWNSGSLSSGVHTFPAVLQSDLYTLSSSVTVPSSTWPSETVTCN

SEQ ID NO: 29 - protein, 193 aa, Murine
LKPSQSLSLTCSVTGYSITGGYYWNWIRQFPGNKLEWMGYIRYDGSNNYNPSLKNRISITRDTSKNQFFLKLNSVTTEDTATYYCARGGYDGLYYAMDYWGQGTSVTVSSAKTTAPSVYPLAPVCGDTTGSSMTLGCLVKGYFPEPVTLTWNSGSLSSGVHTFPAVLQSDLYTLSSSVTVTSSTWPSQSITCN

SEQ ID NO: 30 - protein, 157 aa, Murine
PGAELVKPGASVKLSCTASGFNIKDTFLHWVKQRPEQGLEWIGRIDPAKDDTKYDPKLQGKATMTADTSSNTAYLQLSSLTSEDTAVYYCARSTLGRAFAYWGQGTLVTVSAAKTTAPSVYPLAPVYGDTTGSSVTLGCLVKGYFPEPVTLTWNSGS

SEQ ID NO: 31 - protein, 199 aa, Murine
LEVQLQQSGAELARPGASVKMSCKASGNTFNTIHWIKQRPGQGLEWIGYINPSNGLTKNNQKFKDKATLTADKSSSTAYMQLSSLTSEDSAVYSCALGYFYAMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCN

SEQ ID NO: 32 - protein, 199 aa, Murine; identical to SEQ ID NO: 31

SEQ ID NO: 33 - protein, 11 aa, Murine
RASQDISNYLN

SEQ ID NO: 34 - protein, 10 aa, Murine
SASSSVSYMY

SEQ ID NO: 35 - protein, 15 aa, Murine
KASQSVDYDGDNYMN

SEQ ID NO: 36 - protein, 11 aa, Murine
KASQNVGTNVA

SEQ ID NO: 37 - protein, 10 aa, Murine
RASSSVSYMY

SEQ ID NO: 38 - protein, 15 aa, Murine
RASESVDNYGISFMN

SEQ ID NO: 39 - protein, 17 aa, Murine
KSSQSLLYSSNQKNYLA

SEQ ID NO: 40 - protein, 11 aa, Murine
RASENIYSNLA

SEQ ID NO: 41 - protein, 11 aa, Murine
KASQNVGTNVV

SEQ ID NO: 42 - protein, 15 aa, Murine
KASQSVDNDGISYMN

SEQ ID NO: 43 - protein, 12 aa, Murine
RASSSVGSSYLH

SEQ ID NO: 44 - protein, 7 aa, Murine
YTSRLHS

SEQ ID NO: 45 - protein, 7 aa, Murine
DTSNLAS

SEQ ID NO: 46 - protein, 7 aa, Murine
AASNLES

SEQ ID NO: 47 - protein, 7 aa, Murine
SASYRYS

SEQ ID NO: 48 - protein, 7 aa, Murine
AASNQGS

SEQ ID NO: 49 - protein, 7 aa, Murine
WASTRES

SEQ ID NO: 50 - protein, 7 aa, Murine
AATNLAD

SEQ ID NO: 51 - protein, 7 aa, Murine
SASYRFG

SEQ ID NO: 52 - protein, 7 aa, Murine
AASNLGS

SEQ ID NO: 53 - protein, 7 aa, Murine
STSKLAS

SEQ ID NO: 54 - protein, 9 aa, Murine
QQGNTLPYT

SEQ ID NO: 55 - protein, 9 aa, Murine
QQWSSYPLT

SEQ ID NO: 56 - protein, 9 aa, Murine
QQSDEDPYT

SEQ ID NO: 57 - protein, 9 aa, Murine
QQGNTLPWT

SEQ ID NO: 58 - protein, 9 aa, Murine
QQYNSYPRT

SEQ ID NO: 59 - protein, 9 aa, Murine
QQYNSYPLT

SEQ ID NO: 60 - protein, 9 aa, Murine
QQWSGYPLT

SEQ ID NO: 61 - protein, 9 aa, Murine
QQSNGDPWT

SEQ ID NO: 62 - protein, 9 aa, Murine
QQTKEVPYT

SEQ ID NO: 63 - protein, 9 aa, Murine
QQYYSYPFT

SEQ ID NO: 64 - protein, 9 aa, Murine
QHFWGTPWT

SEQ ID NO: 65 - protein, 9 aa, Murine
QQYNIYPYT

SEQ ID NO: 66 - protein, 9 aa, Murine
QQYNGYPYT

SEQ ID NO: 67 - protein, 9 aa, Murine
QQYSGYPLT

SEQ ID NO: 68 - protein, 10 aa, Murine
GYTFSSYWIE

SEQ ID NO: 69 - protein, 10 aa, Murine
GYSFANYWMH

SEQ ID NO: 70 - protein, 10 aa, Murine
GYTFTNYYMH

SEQ ID NO: 71 - protein, 10 aa, Murine
GYTFTSYYMY

SEQ ID NO: 72 - protein, 10 aa, Murine
GFNIKDTYIH

SEQ ID NO: 73 - protein, 10 aa, Murine
GYTFTEYTMH

SEQ ID NO: 74 - protein, 10 aa, Murine
GYSFTSYWMH

SEQ ID NO: 75 - protein, 10 aa, Murine
GFTFSSSWIE

SEQ ID NO: 76 - protein, 10 aa, Murine
GFSITGYYMH

SEQ ID NO: 77 - protein, 11 aa, Murine
GYSITGGYYWN

SEQ ID NO: 78 - protein, 10 aa, Murine
GFNIKDTFLH

SEQ ID NO: 79 - protein, 8 aa, Murine
GNTFNTIH

SEQ ID NO: 80 - protein, 17 aa, Murine
EILPGIGTTHYNERFKG

SEQ ID NO: 81 - protein, 17 aa, Murine
AIYPGNTDTSYNQKFKG

SEQ ID NO: 82 - protein, 17 aa, Murine
EINPSSGGTNFNEKFKS

SEQ ID NO: 83 - protein, 17 aa, Murine
EINPSHGGTNFNEKFKN

SEQ ID NO: 84 - protein, 17 aa, Murine
RIDPADGNTKYDPKFQD

SEQ ID NO: 85 - protein, 17 aa, Murine
RIDPADGNTKYDPKFQG

SEQ ID NO: 86 - protein, 17 aa, Murine
GINPNNGGTNYNQKFKG

SEQ ID NO: 87 - protein, 17 aa, Murine
SIYPGNSDTSYNQKFKG

SEQ ID NO: 88 - protein, 17 aa, Murine
EISPGSGSTNFNENFKG

SEQ ID NO: 89 - protein, 17 aa, Murine
YISSYSLATDYNQNFKG

SEQ ID NO: 90 - protein, 16 aa, Murine
YIRYDGSNNYNPSLKN

SEQ ID NO: 91 - protein, 17 aa, Murine
RIDPAKDDTKYDPKLQG

SEQ ID NO: 92 - protein, 17 aa, Murine
YINPSNGLTKNNQKFKD

SEQ ID NO: 93 - protein, 8 aa, Murine
KNYDWFAY

SEQ ID NO: 94 - protein, 7 aa, Murine
LRPPFNF

SEQ ID NO: 95 - protein, 10 aa, Murine
FDRTENGMDY

SEQ ID NO: 96 - protein, 11 aa, Murine
GGNYPYFAMDY

SEQ ID NO: 97 - protein, 8 aa, Murine
AFYYSMDY

SEQ ID NO: 98 - protein, 7 aa, Murine
WTGDFDV

SEQ ID NO: 99 - protein, 10 aa, Murine
FDRTENGLDY

SEQ ID NO: 100 - protein, 11 aa, Murine
FYGNNLYYFDY

SEQ ID NO: 101 - protein, 12 aa, Murine
GDYASPYWFFDV

SEQ ID NO: 102 - protein, 12 aa, Murine
GGYDGLYYAMDY

SEQ ID NO: 103 - protein, 9 aa, Murine
STLGRAFAY

SEQ ID NO: 104 - protein, 8 aa, Murine
GYFYAMDY

SEQ ID NO: 105 - protein, 97 aa, Artificial Sequence
  humanized mouse antibody
DIQMTQTTSSLSASLGDRVTISCRASQDISNYLNWYQQKPDGTVKLLIYYTSRLHSGVPSRFSGSGSGTDYSLTISNLEQEDIATYFCQQGNTLPWT

SEQ ID NO: 106 - protein, 119 aa, Artificial Sequence
  humanized mouse antibody
VQLQQPGAELVKPGASVKMSCKASGYTFTSYYMYWVKQRPGQGLEWIGEINPSHGGTNFNEKFKNKATLTVDKSSSTVYMQLSSLTSEDSAVYYCTRGGNYPYFAMDYWGQGTSVTVSS

SEQ ID NO: 107 - protein, 214 aa, Artificial Sequence
  humanized mouse antibody
DIQMTQTTSSLSASLGDRVTISCRASQDISNYLNWYQQKPDGTVKLLIYYTSRLHSGVPSRFSGSGSGTDYSLTISNLEQEDIATYFCQQGNTLPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 108 - protein, 449 aa, Artificial Sequence
  humanized mouse antibody
VQLQQPGAELVKPGASVKMSCKASGYTFTSYYMYWVKQRPGQGLEWIGEINPSHGGTNFNEKFKNKATLTVDKSSSTVYMQLSSLTSEDSAVYYCTRGGNYPYFAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK